(19) United States
(12) Patent Application Publication
(10) Pub. No.: US 2022/0257533 A1
(43) Pub. Date: Aug. 18, 2022

(54) METHODS AND COMPOSITIONS FOR TREATMENT OF INFLAMMATION AND OXIDATIVE STRESS

(71) Applicant: PTC Therapeutics, Inc., South Plainfield, NJ (US)

(72) Inventors: Guy M. MILLER, Monte Sereno, CA (US); Jeffery K. TRIMMER, San Carlos, CA (US); William SHRADER, Belmont, CA (US); Andrew W. HINMAN, San Francisco, CA (US); Charles R. HOLST, San Jose, CA (US); Joey C. LATHAM, San Carlos, CA (US); Joel J. BRUEGGER, Campbell, CA (US); Kevin P. MCCUSKER, Berkeley, CA (US); Gözde ULAS, Sunnyvale, CA (US); Dana DAVIS, Sunnyvale, CA (US); Amanda H. KAHN-KIRBY, San Francisco, CA (US); Edgar P. LEE, Mountain View, CA (US); Stephanie A. MALONE, San Francisco, CA (US); Steven J. RICHARDS, Brisbane, CA (US); Matthew B. KLEIN, Menlo Park, CA (US)

(21) Appl. No.: 17/537,260

(22) Filed: Nov. 29, 2021

Related U.S. Application Data
(63) Continuation of application No. 16/605,192, filed on Oct. 14, 2019, now abandoned.
(60) Provisional application No. 62/635,470, filed on Feb. 26, 2018.
(60) Provisional application No. 62/572,406, filed on Oct. 13, 2017.
(60) Provisional application No. 62/534,649, filed on Jul. 19, 2017.
(60) Provisional application No. 62/531,866, filed on Jul. 12, 2017.
(60) Provisional application No. 62/488,643, filed on Apr. 21, 2017.
(60) Provisional application No. 62/488,658, filed on Apr. 21, 2017.
(60) Provisional application No. 62/485,739, filed on Apr. 14, 2017.
(60) Provisional application No. 62/485,832, filed on Apr. 14, 2017.

(51) Int. Cl. A61K 31/122; A61P 29/00

(52) U.S. Cl. CPC A61K 31/122; A61P 29/00

(57) ABSTRACT

Compounds, compositions, and methods for treatment of, or prophylaxis against, inflammation and/or oxidative stress are disclosed.

### CROSS-REFERENCE TO RELATED APPLICATIONS

This application claims priority to U.S. Provisional Application No. 62/485,739 filed on Apr. 14, 2017 and titled “VITAMIN E COMPOSITIONS AND METHODS OF USE THEREFOR”; U.S. Provisional Application No. 62/488,643 filed on Apr. 21, 2017 and titled “VITAMIN E COMPOSITIONS AND METHODS OF USE THEREFOR”; U.S. Provisional Application No. 62/485,832 filed on Apr. 14, 2017 and titled “METHODS AND COMPOSITIONS FOR TREATMENT OF INFLAMMATION AND OXIDATIVE STRESS”; U.S. Provisional Application No. 62/488,658 filed on Apr. 21, 2017 and titled “METHODS AND COMPOSITIONS FOR TREATMENT OF INFLAMMATION AND OXIDATIVE STRESS”; U.S. Provisional Application No. 62/531,866 filed on Jul. 12, 2017 and titled “METHODS AND COMPOSITIONS FOR TREATMENT OF INFLAMMATION AND OXIDATIVE STRESS”; U.S. Provisional Application No. 62/534,649 filed on Jul. 19, 2017 and titled “METHODS AND COMPOSITIONS FOR TREATMENT OF INFLAMMATION AND OXIDATIVE STRESS”; U.S. Provisional Application No. 62/572,406 filed on Oct. 13, 2017 and titled “METHODS AND COMPOSITIONS FOR TREATMENT OF INFLAMMATION AND OXIDATIVE STRESS”; U.S. Provisional Application No. 62/635,470 filed on Feb. 26, 2018, and titled “METHODS, COMPOSITIONS AND SYSTEMS FOR MONITORING AND INFLUENCING FERROPTOSIS AND INFLAMMATION IN BIOLOGICAL SYSTEMS”; each of which is hereby incorporated by reference in its entirety.

### TECHNICAL FIELD

The application discloses compositions and methods useful for treating or protecting biological systems against damage caused by inflammation and/or oxidative stress.

### BACKGROUND

Biological systems experiencing inflammation or oxidative stress can benefit from compositions and compounds acting to reduce inflammation and to interrupt the propagation of reactive oxygen species. Inflammation within the biological systems can be treated with various anti-inflammatory compounds such as aspirin, ibuprofen, indomethacin and others. Oxidative stress within the biological system oftentimes will involve reducing the concentration of free radicals, as these free radicals are disruptive in reactions within cells, tissues, and organisms. While inflammation and oxidative stress define regimes which can have some overlapping areas, previous treatment of these regimes would typically entail distinct treatments. The various mechanisms causing inflammatory or oxidative stress episodes have not been viewed as having any common treatment approach, and to date, insights into mechanistic causes of these issues has not yet yielded any commonality in treatment.

There is thus a need for protective agents that can mitigate the adverse effects of inflammation and/or oxidative stress upon biological systems.

### SUMMARY OF THE INVENTION

The present disclosure provides, in some embodiments, compounds and compositions for use in treating or protecting against injury or damage caused by inflammation and/or oxidative stress, and methods of using such compounds for treating or for protecting against injury or damage caused by inflammation and/or oxidative stress. In certain embodiments the inflammation is in endothelial cells/tissue. In certain instances the inflammation is a drug induced injury. Provided is a composition comprising one or more compounds for treating an indicator of inflammation and/or oxidative stress in a biological system, the one or more compounds comprising:

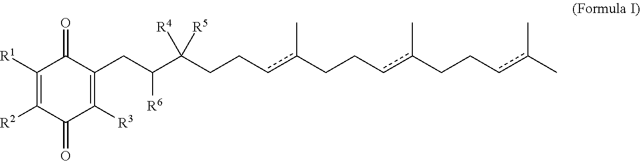

wherein: each bond indicated with a dashed line is independently a single bond or a double bond; R¹, R², and R³ are independently selected from H(C₁-C₄)-alkyl, (C₁-C₄)-haloalkyl, —CN, —F, —Cl, —Br, and —I; and R⁴ and R⁵ are independently selected from hydroxy and (C₁-C₄)-alkyl, and R⁶ is hydrogen; or R⁴ is (C₁-C₄)-alkyl, and R⁵ and R⁶ are hydrogen; or R⁴ is (C₁-C₄)-alkyl, and R⁵ and R⁶ together form the second bond of a double bond between the carbon atoms to which they are attached; or a stereoisomer, mixtures of stereoisomers, hydroquinone form, prodrug, metabolite, salt, phosphate substituted form, crystalline form, non-crystalline form, isotopologue, deuterated form, hydrate, or solvate thereof. In one instance the one or more compounds is selected from the group consisting of alpha-tocotrienol quinone, beta-tocotrienol quinone, gamma-tocotrienol quinone, delta-tocotrienol quinone, alpha-tocopherol quinone, beta-tocopherol quinone, gamma-tocopherol quinone, delta-tocopherol quinone, and stereoisomers, mixtures of stereoisomers, hydrates, and solvates thereof. In one instance the one or more compounds is selected from the group consisting of alpha-tocotrienol quinone, and stereoisomers, mixtures of stereoisomers, hydrates, and solvates thereof. In one instance the one or more compounds is selected from the group consisting of alpha-tocopherol quinone, and stereoisomers, mixtures of stereoisomers, hydrates, and solvates thereof. In an instance an additional material is co-administered with the one or more compounds, the additional material selected from the group consisting of N-acetyl cysteine (NAC) and Docosahexaenoic acid (DHA), preferably where the additional material is DHA. In an instance the additional material has a weight % concentration of 1-99% of the one or more compounds. In some cases, the indicator is for a liver disease, kidney disease, skin disease, or eye disease. In some cases, the indicator for the liver disease is from a disease state selected from the group consisting of NASH/NAFL, alcoholic hepatitis, cholestatic liver disease, viral hepatitis, drug-induced liver toxicity, hemachromatosis, Wilson's disease, transplant reperfusion injury, and hepatic insufficiency where the hepatic insufficiency is due to injury, SIRS, sepsis, and severe illness. In some cases the liver is undergoing inflammation and/or oxidative stress due to drug-induced liver toxicity.

In another aspect is provided a method of mitigating inflammation and/or oxidative stress in a biological system comprising: administering one or more compounds or administering a composition comprising one or more compounds, to the biological system experiencing inflammation or oxidative stress, wherein the one or more compounds comprise:

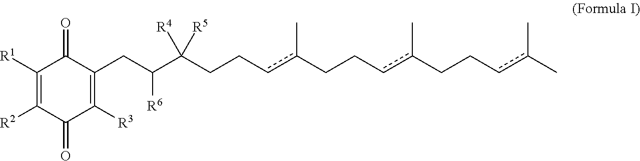

wherein: each bond indicated with a dashed line is independently a single bond or a double bond; R¹, R², and R³ are independently selected from H, (C₁-C₄)-alkyl, (C₁-C₄)-haloalkyl, —CN, —F, —Cl, —Br, and —I; and R⁴ and R⁵ are independently selected from hydroxy and (C₁-C₄)-alkyl, and R⁶ is hydrogen; or R⁴ is (C₁-C₄)-alkyl, and R⁵ and R⁶ are hydrogen; or R⁴ is (C₁-C₄)-alkyl, and R⁵ and R⁶ together form the second bond of a double bond between the carbon atoms to which they are attached; or a stereoisomer, mixtures of stereoisomers, hydroquinone form, prodrug, metabolite, salt, phosphate substituted form, crystalline form, non-crystalline form, isotopologue, deuterated form, hydrate, or solvate thereof. In some cases the one or more compounds is selected from the group consisting of alpha-tocotrienol quinone, beta-tocotrienol quinone, gamma-tocotrienol quinone, delta-tocotrienol quinone, alpha-tocopherol quinone, beta-tocopherol quinone, gamma-tocopherol quinone, delta-tocopherol quinone, and stereoisomers, mixtures of stereoisomers, hydrates, and solvates thereof. In some cases the one or more compounds is selected from the group consisting of alpha-tocotrienol quinone, and stereoisomers, mixtures of stereoisomers, hydrates, and solvates thereof. In some cases the one or more compounds is selected from the group consisting of alpha-tocopherol quinone, and stereoisomers, mixtures of stereoisomers, hydrates, and solvates thereof. In some instances administering the one or more compounds is done orally. In some cases administering the one or more compounds is achieved in a medical food. In certain instances administering the one or more compounds is achieved in an ingestible supplement. In some instances administering the one or more compounds is done via injection. In some instances administering the one or more compounds is done topically. In some instances administering is done in conjunction with a member selected from the group consisting of NAC and DHA, preferably where the additional material is DHA. In some instances the member has a weight % concentration of 1-99% of the one or more compounds. In some instances the biological system is a eukaryotic system. In some instances the biological system is a human subject. In some instances the administering comprises about 100 mg to about 500 mg/kg of the one or more compounds per kg of body weight. In some instances the administering comprises about 100 mg to about 300 mg/kg of the one or more compounds per kg of body weight. In some instances the administering comprises about 100 mg to about 200 mg/kg of the one or more compounds per kg of body weight. In some instances the human subject is selected from the group consisting of a patient undergoing one or more inflammation and/or oxidative stress symptoms associated with a liver disease, kidney disease, eye disease, or skin disease. In some instances the one or more symptoms associated with the liver disease are from disease states selected from the group consisting of NASH/NAFL, alcoholic hepatitis, cholestatic liver disease, viral hepatitis, drug-induced liver toxicity, hemachromatosis, Wilson's disease, transplant reperfusion injury, and hepatic insufficiency. In some instances the patient is undergoing inflammation and/or oxidative stress due to hepatic insufficiency where the hepatic insufficiency is due to injury, SIRS, sepsis, or severe illness. In some instances the patient is undergoing inflammation and/or oxidative stress due to drug-induced liver toxicity. In some instances for any composition or method the inflammation and/or oxidative stress is not associated with diabetes, liver damage, insulin resistance, nephropathy, acute or chronic renal injury due to exposure to nephrotoxic agents, drug induced peripheral neuropathy, serious atopic dermatitis, psoriasis, eczema, cyanide poisoning, decreased renal function, acute lung injury, calciphylaxis in blood vessels, kidney toxicity in cancer therapy, bacterial infection, fungal infection, ulcerative colitis, inflammatory bowel disease, hypertension, proteinuria, ischemia, hypoxia, atherosclerosis, uterus obstruction, hyperalgesia, lactic acidosis, Kearns-Sayre Syndrome (KSS), and primary progressive multiple sclerosis. In some instances for any composition or method the inflammation and/or oxidative stress is not associated with diabetes, liver damage, and insulin resistance. In some instances for any composition or method the inflammation and/or oxidative stress is not associated with diabetes. In some instances for any composition or method the inflammation and/or oxidative stress is not associated with liver damage. In some instances for any composition or method the inflammation and/or oxidative stress is not associated with insulin resistance. In some instances for any composition or method the inflammation and/or oxidative stress is not associated with inflammatory bowels disease. In some instances for any composition or method the inflammation and/or oxidative stress is not associated with cyanide poisoning, decreased renal function, acute lung injury, calciphylaxis in blood vessels, kidney toxicity in cancer therapy, bacterial infection, fungal infection, ulcerative colitis, hypertension, and proteinuria. In some instances for any composition or method the inflammation and/or oxidative stress is not related to an increased amount of thiosulfate in the tissue or human to which the administering is administered. In some instances for any composition or method mean liver enzyme(s) concentration(s) in serum are reduced. In some instances for any composition or method liver enzyme(s) concentration(s) in serum are reduced. In some instances for any composition or method the serum concentration of pro-inflammatory cytokine(s) is suppressed. In some instances for any composition or method the pro-inflammatory cytokines are selected from the group consisting of IL-6, IL-8 and MCP-1.In some instances for any composition or method the suppressing is dose dependent. In some instances for any composition or method the one or more compounds is a synthetic, non-plant based, substantially pure stereoisomer of 1) an alpha, beta, gamma, or delta tocopherol quinone, or hydroquinone thereof, or 2) an alpha, beta, gamma, or delta tocotrienol quinone, or hydroquinone thereof, wherein the stereoisomer is selected from the group consisting of: (R,R,R)-alpha-tocopherol quinone, (R,R,R)-beta-tocopherol quinone, (R,R,R)-gamma-tocopherol quinone, (R,R,R)-delta-tocopherol quinone, (R,E,E)-alpha-tocotrienol quinone, (R,E,E)-beta-tocotrienol quinone, (R,E,E)-gamma-tocotrienol quinone, (R,E,E)-delta-tocotrienol quinone, (R,R,R)-alpha-tocopherol hydroquinone, (R,R,R)-beta-tocopherol hydroquinone, (R,R,R)-gamma-tocopherol hydroquinone, (R,R,R)-delta-tocopherol hydroquinone, (R,E,E)-alpha-tocotrienol hydroquinone, (R,E,E)-beta-tocotrienol hydroquinone, (R,E,E)-gamma-tocotrienol hydroquinone, and (R,E,E)-delta-tocotrienol hydroquinone. In some instances for any composition or method the stereoisomer is (R,R,R)-alpha-tocopherol quinone. In some instances for any composition or method the stereoisomer is (R,R,R)-beta-tocopherol quinone. In some instances for any composition or method the stereoisomer is (R,R,R)-gamma-tocopherol quinone. In some instances for any composition or method the stereoisomer is (R,R,R)-delta-tocopherol quinone. In some instances for any composition or method the stereoisomer is (R,E,E)-alpha-tocotrienol quinone. In some instances for any composition or method the stereoisomer is (R,E,E)-beta-tocotrienol quinone. In some instances for any composition or method the stereoisomer is (R,E,E)-gamma-tocotrienol quinone. In some instances for any composition or method the stereoisomer is (R,E,E)-delta-tocotrienol quinone. In some instances for any composition or method the stereoisomer is (R,R,R)-alpha-tocopherol hydroquinone. In some instances for any composition or method the stereoisomer is (R,R,R)-beta-tocopherol hydroquinone. In some instances for any composition or method the stereoisomer is (R,R,R)-gamma-tocopherol hydroquinone. In some instances for any composition or method the stereoisomer is (R,R,R)-delta-tocopherol hydroquinone. In some instances for any composition or method the stereoisomer is (R,E,E)-alpha-tocotrienol hydroquinone. In some instances for any composition or method the stereoisomer is (R,E,E)-beta-tocotrienol hydroquinone. In some instances for any composition or method the stereoisomer is (R,E,E)-gamma-tocotrienol hydroquinone. In some instances for any composition or method the stereoisomer is (R,E,E)-delta-tocotrienol hydroquinone. In some instances for any composition or method the substantially pure stereoisomer comprises at least about 0.001% by weight of the composition. In some instances for any composition or method the substantially pure stereoisomer comprises at least about 0.01% by weight of the composition. In some instances for any composition or method the substantially pure stereoisomer comprises at least about 0.1% by weight of the composition. In some instances for any composition or method the substantially pure stereoisomer comprises at least about 1.0% by weight of the composition. In some instances for any composition or method the substantially pure stereoisomer comprises at least about 10.0% by weight of the composition. In some instances for any composition or method the kidney disease is not nephropathy, acute or chronic renal injury due to exposure to nephrotoxic agents, or drug induced peripheral neuropathy. In some instances for any composition or method the skin disease is not serious atopic dermatitis, psoriasis, and eczema. In some instances for any composition or method the biological system is a human and the human is an adult. In some instances for any composition or method the biological system is a human and the human is a child. In some instances for any composition or method the biological system is a human and the human is male. In some instances for any composition or method the biological system is a human and the human is female. In some instances for any composition or method the mean liver enzyme(s) concentration(s) in serum are reduced by at least 40%, 50%, 60%, 70%, 80%, 90%, or 100%. In some instances for any composition or method the liver enzyme(s) concentration(s) in serum are reduced to a concentration less than 3200 U/L, less than 3000 U/L, less than 2800 U/L, less than 2600 U/L, less than 2400 U/L, less than 2200 U/L, less than 2000 U/L, less than 1800 U/L, less than 1600 U/L, less than 1400 U/L, less than 1200 U/L, less than 1000 U/L, less than 800 U/L, less than 600 U/L, less than 400 U/L, less than 200 U/L, or less than 100 U/L. In some instances for any composition or method serum concentration of pro-inflammatory cytokine(s) are reduced. In some instances for any composition or method serum concentration of pro-inflammatory cytokine(s) are reduced by at least 40%, 50%, 60%, 70%, 80%, 90%, or 100%. In some instances for any composition or method the pro-inflammatory cytokines are selected from the group consisting of IL-6, IL-8 and MCP-1. In some instances for any composition, the composition further comprises one or more compounds selected from the group consisting of: (R,R,R)-alpha-tocopherol, (R,R,R)-beta-tocopherol, (R,R,R)-gamma-tocopherol, (R,R,R)-delta-tocopherol, (R,E,E)-alpha-tocotrienol, (R,E,E)-beta-tocotrienol, (R,E,E)-gamma-tocotrienol, and (R,E,E)-delta-tocotrienol. In some instances for any composition or method the stereoisomer is (R,R,R)-alpha-tocopherol. In some instances for any composition or method the stereoisomer is (R,R,R)-beta-tocopherol. In some instances for any composition or method the stereoisomer is (R,R,R)-gamma-tocopherol. In some instances for any composition or method the stereoisomer is (R,R,R)-delta-tocopherol. In some instances for any composition or method the stereoisomer is (R,E,E)-alpha-tocotrienol. In some instances for any composition or method the stereoisomer is (R,E,E)-beta-tocotrienol. In some instances for any composition or method the stereoisomer is (R,E,E)-gamma-tocotrienol. In some instances for any composition or method the stereoisomer is (R,E,E)-delta-tocotrienol. In some instances for any composition or method the (R,R,R)-alpha-tocopherol, (R,R,R)-beta-tocopherol, (R,R,R)-gamma-tocopherol, (R,R,R)-delta-tocopherol, (R,E,E)-alpha-tocotrienol, (R,E,E)-beta-tocotrienol, (R,E,E)-gamma-tocotrienol, and (R,E,E)-delta-tocotrienol are a synthetic, non-plant based, substantially pure stereoisomer. In some instances for any composition, the composition further comprises DHA.

In one aspect is a compound of the formula:

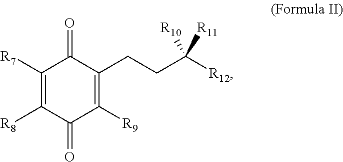

or the hydroquinone form thereof wherein: R₇, R₈, and R9 are independently hydrogen, C₁-C₆ alkyl, or C₁-C₆ alkoxy; R₁₀ and R₁₁ are independently selected from the group consisting of hydrogen, methyl, and hydroxyl, with the proviso that only one of R₁₀ and R₁₁ may be hydroxyl; and R₁₂ is a C₁-C₁₃-alkyl or C₂-C₁₃-alkenyl group, wherein the C₁-C₁₃-alkyl or C₂-C₁₃-alkenyl groups are optionally substituted with one or more groups selected from the group consisting of: C₁-C₃ alkyl, halo, C₁-C₆ alkoxy, C₁-C₆ haloalkoxy, hydroxyl, and carboxylic acid; or a stereoisomer, mixtures of stereoisomers, salt, phosphate substituted form, crystalline form, non-crystalline form, isotopologue, deuterated form, hydrate, or solvate thereof. In some embodiments, R₇, R₈, and R₉ are independently hydrogen, C₁-C₃ alkyl, or C₁-C₃ alkoxy. In some embodiments, R₇, R₈, and R₉ are independently hydrogen, methyl, or methoxy. In some embodiments, R₇, R₈, and R₉ are independently hydrogen or methyl. In some embodiments, R₇, R₈, and R₉ are methyl. In some embodiments, including any of the foregoing embodiments, one of R₁₀ and R₁₁ is hydroxyl and the other is methyl. In some embodiments, including any of the foregoing embodiments, R₁₀ and R₁₁ are both hydrogen. In some embodiments, including any of the foregoing embodiments, the C₁-C₁₃-alkyl or C₂-C₁₃-alkenyl groups are substituted with one or more groups selected from the group consisting of: C₁-C₃ alkyl, halo, C₁-C₆ alkoxy, C₁-C₆ haloalkoxy, hydroxyl, and carboxylic acid groups. In some embodiments, including any of the foregoing embodiments, the C₁-C₁₃-alkyl or C₂-C₁₃-alkenyl groups are substituted with one or more methyl groups. In some embodiments, including any of the foregoing embodiments, R₁₂ is C₁-C₁₃-alkyl substituted with one or more halo groups. In some embodiments, including any of the foregoing embodiments, R₁₂ is C₁-C₁₃-alkyl substituted with C₁-C₆ alkoxy. In some embodiments, including any of the foregoing embodiments, R₁₂ is C₁-C₁₃-alkyl substituted with C₁-C₄ alkoxy. In some embodiments, including any of the foregoing embodiments, R₁₂ is C₁-C₁₃-alkyl substituted with C₁-C₃ haloalkoxy. In some embodiments, including any of the foregoing embodiments, the C₁-C₁₃-alkyl or C₂-C₁₃-alkenyl groups are unsubstituted. In some embodiments, including any of the foregoing embodiments, R₁₂ is unsubstituted C₅-C₉-alkyl. In some embodiments, including any of the foregoing embodiments, the compound is in the quinone form. In some embodiments, including any of the foregoing embodiments, the compound is in the hydroquinone form. In some embodiments, the compound is combined with a drug that may cause solid organ injury, as a pharmaceutical formulation. In some embodiments, the solid organ is liver. In some embodiments, the drug is acetaminophen.

In another aspect, provided are compositions comprising one or more quinone or hydroquinone compounds as described herein, docosahexaenoic acid (DHA), and one or more compounds selected from the group consisting of: alpha tocopherol, beta tocopherol, gamma tocopherol, delta tocopherol, alpha tocotrienol, beta tocotrienol, gamma tocotrienol, and delta tocotrienol.

In another aspect are compositions comprising one or more quinone or hydroquinone compounds as described herein, N-acetyl cysteine (NAC), and one or more compounds selected from the group consisting of: alpha tocopherol, beta tocopherol, gamma tocopherol, delta tocopherol, alpha tocotrienol, beta tocotrienol, gamma tocotrienol, and delta tocotrienol.

In a further aspect are provided methods of using one or more compounds of Formula I and/or Formula II, or compositions of one or more compounds of Formula I and/or Formula II disclosed herein, or a stereoisomer, mixtures of stereoisomers, hydroquinone form, prodrug, metabolite, salt, phosphate substituted form, crystalline form, non-crystalline form, isotopologue, deuterated form, hydrate, or solvate thereof, for treating or for protecting against injury or damage caused by inflammation and/or oxidative stress, comprising administering to a cell or cells, a tissue or tissues, or a subject in need thereof, a therapeutically effective amount or a prophylactically effective amount of a compound or composition disclosed herein. In some embodiments, the one or more compounds are used therapeutically during, after, or during and after exposure to a factor which may result in inflammation and/or oxidative stress. In some embodiments, the one or more compounds are used prophylactically prior to, or in conjunction with, exposure to a factor which may result in an indication of inflammation and/or oxidative stress. In some embodiments, the one or more compounds are administered concurrently with onset of inflammation and/or oxidative stress. In some embodiments, the one or more compounds are administered after onset of inflammation and/or oxidative stress. Additional aspects and advantages of the present disclosure will become readily apparent to those skilled in this art from the following detailed description, wherein only illustrative cases of the present disclosure are shown and described. As will be realized, the present disclosure is capable of other and different cases, and its several details are capable of modifications in various obvious respects, all without departing from the present disclosure. Accordingly, the drawings and description are to be regarded as illustrative in nature, and not as restrictive.

### BRIEF DESCRIPTION OF THE DRAWINGS

FIG. 1 illustrates Alanine Transaminase (ALT) enzyme levels at 4 hours for a control and Acetaminophen (APAP) introduction, where APAP introduction is made in the presence of distinct test compounds.

FIG. 2 illustrates Aspartate Transaminase (AST) enzyme levels at 4 hours for a control and APAP introduction, where APAP introduction is made in the presence of distinct test compounds.

FIG. 3 illustrates ALT enzyme levels at 24 hours for a control and APAP introduction, where APAP introduction is made in the presence of distinct test compounds.

FIG. 4 illustrates AST enzyme levels at 24 hours for a control and APAP introduction, where APAP introduction is made in the presence of distinct test compounds.

FIG. 5 illustrates IL-6 levels at 6 hours for Donor 1 for a control and various dosing levels of test compounds.

FIG. 6 illustrates IL-6 levels at 6 hours for Donor 2 for a control and various dosing levels of test compounds.

FIG. 7 illustrates normalized IL-6 levels at 6 hours for Donor 486 using various dosing levels of test compounds.

FIG. 8 illustrates normalized IL-8 levels at 6 hours for Donor 486 using various dosing levels of test compounds.

FIG. 9 illustrates normalized TNFₐ levels at 6 hours for Donor 486 using various dosing levels of test compounds.

FIG. 10 illustrates normalized IL-6 levels at 6 hours for Donor 662 using various dosing levels of test compounds.

FIG. 11 illustrates normalized IL-8 levels at 6 hours for Donor 662 using various dosing levels of test compounds.

FIG. 12 illustrates normalized TNFₐ levels at 6 hours for Donor 662 using various dosing levels of test compounds.

FIG. 13 illustrates normalized ATP levels at 6 hours for Donor 486 using various dosing levels of test compounds.

FIG. 14 illustrates normalized ATP levels at 6 hours for Donor 662 using various dosing levels of test compounds.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

U.S. Provisional Application No. 62/485,739, titled “VITAMIN E COMPOSITIONS AND METHODS OF USE THEREFOR”, filed on Apr. 14, 2017; U.S. Provisional Application No. 62/488,658; and U.S. Provisional Application No. 62/488,643, titled “VITAMIN E COMPOSITIONS AND METHODS OF USE THEREFOR”, filed on Apr. 21, 2017, U.S. Provisional Application No. 62/635,470 filed on Feb. 26, 2018, and titled “METHODS, COMPOSITIONS AND SYSTEMS FOR MONITORING AND INFLUENCING FERROPTOSIS AND INFLAMMATION IN BIOLOGICAL SYSTEMS”, U.S. Non-Provisional Application No. titled “VITAMIN E COMPOSITIONS AND METHODS OF USE THEREFOR”, filed on Apr. 14, 2018, all by the same Applicant, are incorporated by reference herein in their entireties. all by the same Applicant, are incorporated by reference herein in their entireties.

### DETAILED DESCRIPTION

The present disclosure provides, in some embodiments, compounds or compositions and methods of using one or more compounds or compositions of one or more compounds as set forth below. Although the compounds are described herein throughout in their quinone (oxidized) form, the hydroquinone (reduced) form of the compound may be used where desired.

In some embodiments of the disclosure, provided is a composition comprising one or more compounds for preventing or treating one or more indicators of inflammation and/or oxidative stress in a biological system, the one or more compounds comprising:

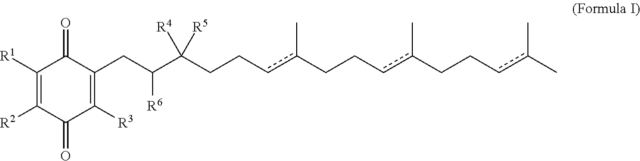

wherein:
each bond indicated with a dashed line is independently a single bond or a double bond;
R¹, R², and R³ are independently selected from H, (C₁-C₄)-alkyl, (C₁-C₄)-haloalkyl, —CN, —F, —Cl, —Br, and —I; and
R⁴ and R⁵ are independently selected from hydroxy and (C₁-C₄)-alkyl, and R⁶ is hydrogen; or
R⁴ is (C₁-C₄)-alkyl, and R⁵ and R⁶ are hydrogen; or
R⁴ is (C₁-C₄)-alkyl, and R⁵ and R⁶ together form the second bond of a double bond between the carbon atoms to which they are attached; or a stereoisomer, mixtures of stereoisomers, hydroquinone form, prodrug, metabolite, salt, phosphate substituted form, crystalline form, non-crystalline form, isotopologue, deuterated form, hydrate, or solvate thereof.

In some embodiments of the present disclosure, the one or more compounds is selected from the group consisting of alpha-tocotrienol quinone, beta-tocotrienol quinone, gamma-tocotrienol quinone, delta-tocotrienol quinone, alpha-tocopherol quinone, beta-tocopherol quinone, gamma-tocopherol quinone, delta-tocopherol quinone, and their hydroquinone forms thereof, and stereoisomers, mixtures of stereoisomers, hydrates, and solvates thereof.

In some embodiments of the present disclosure, the one or more compounds is selected from the group consisting of alpha-tocotrienol quinone, beta-tocotrienol quinone, gamma-tocotrienol quinone, and delta-tocotrienol quinone, and their hydroquinone forms thereof, and stereoisomers, mixtures of stereoisomers, hydrates, and solvates thereof.

In some embodiments of the present disclosure, the one or more compounds is selected from the group consisting of alpha-tocotrienol quinone, and stereoisomers, mixtures of stereoisomers, hydrates, and solvates thereof.

In some embodiments of the present disclosure, the one or more compounds is selected from the group consisting of alpha-tocopherol quinone, and stereoisomers, mixtures of stereoisomers, hydrates, and solvates thereof.

In some embodiments of the present disclosure, an additional material is co-administered with the one or more compounds, the additional material selected from the group consisting of N-acetyl cysteine (NAC) and docosahexaenoic acid (DHA). In some embodiments, the additional material has a weight % concentration of 1-99% of the one or more compounds.

In some embodiments of the present disclosure, the indicator is representative of inflammation and/or oxidative stress in a biological system for liver diseases, kidney diseases, skin diseases, or eye diseases. In some embodiments of the present disclosure, the indicator is representative of inflammation in a biological system for liver diseases, kidney diseases, skin diseases, or eye diseases. In some embodiments of the present disclosure, the indicator is representative of oxidative stress in a biological system for liver diseases, kidney diseases, skin diseases, or eye diseases. The liver disease states can be selected from the group consisting of NASH/NAFL, alcoholic hepatitis, cholestatic liver disease, viral hepatitis, drug-induced liver toxicity, hemachromatosis, Wilson's disease, transplant reperfusion injury, and hepatic insufficiency where the hepatic insufficiency is due to, in some nonlimiting embodiments, injury (including but not limited to injury to the liver), SIRS, sepsis, and severe illness. In some or any embodiments, as disclosed herein, the liver disease states do not include liver damage or insulin resistance. In some or any embodiments, as disclosed herein, the kidney disease does not include diabetes, nephropathy, acute or chronic renal injury due to exposure to nephrotoxic agents, or drug induced peripheral neuropathy. In some or any embodiments, as disclosed herein, the skin disease does not include serious atopic dermatitis, psoriasis, or eczema.

In some embodiments, the disclosure embraces a method of reducing the effect of inflammation and/or oxidative stress on the biological system using a therapeutically effective amount or a prophylactically effective amount of one or more compounds or composition comprising one or more compounds. In some embodiments, the inflammation does not include inflammatory bowel disease. In some embodiments, the post-treatment mean liver enzyme(s) concentration(s) in serum is reduced to at least about one standard deviation lower than a pre-treatment level. In some embodiments, the post-treatment mean liver enzyme(s) concentration(s) in serum is reduced to at least about two standard deviation lower than a pre-treatment level. In some embodiments, the post-treatment mean liver enzyme(s) concentration(s) in serum is reduced to at least about three standard deviation lower than a pre-treatment level. In some embodiments, the liver enzyme(s) concentration(s) in serum are reduced to a concentration less than 3200 U/L, less than 3000 U/L, less than 2800 U/L, less than 2600 U/L, less than 2400 U/L, less than 2200 U/L, less than 2000 U/L, less than 1800 U/L, less than 1600 U/L, less than 1400 U/L, less than 1200 U/L, less than 1000 U/L, less than 800 U/L, less than 600 U/L, less than 400 U/L, less than 200 U/L, or less than 100 U/L. In some embodiments, the mean liver enzyme(s) concentration(s) in serum are reduced by at least about 10%, 15%, 20%, 25%, 30%, 35%, 40%, 45%, 50%, 55%, 60%, 65%, 70%, 75%, 80%, 85%, 90%, 95%, or 100%. In one embodiment, the mean liver enzyme(s) concentration(s) in serum is reduced by at least about 10%. In another embodiment, the mean liver enzyme(s) concentration(s) in serum is reduced by at least about 15%. In another embodiment, the mean liver enzyme(s) concentration(s) in serum is reduced by at least about 20%. In another embodiment, the mean liver enzyme(s) concentration(s) in serum is reduced by at least about 25%. In another embodiment, the mean liver enzyme(s) concentration(s) in serum is reduced by at least about 30%. In another embodiment, the mean liver enzyme(s) concentration(s) in serum is reduced by at least about 35%. In another embodiment, the mean liver enzyme(s) concentration(s) in serum is reduced by at least about 40%. In another embodiment, the mean liver enzyme(s) concentration(s) in serum is reduced by at least about 45%. In another embodiment, the mean liver enzyme(s) concentration(s) in serum is reduced by at least about 50%. In another embodiment, the mean liver enzyme(s) concentration(s) in serum is reduced by at least about 55%. In another embodiment, the mean liver enzyme(s) concentration(s) in serum is reduced by at least about 60%. In another embodiment, the mean liver enzyme(s) concentration(s) in serum is reduced by at least about 65%. In another embodiment, the mean liver enzyme(s) concentration(s) in serum is reduced by at least about 70%. In another embodiment, the mean liver enzyme(s) concentration(s) in serum is reduced by at least about 75%. In another embodiment, the mean liver enzyme(s) concentration(s) in serum is reduced by at least about 80%. In another embodiment, the mean liver enzyme(s) concentration(s) in serum is reduced by at least about 85%. In another embodiment, the mean liver enzyme(s) concentration(s) in serum is reduced by at least about 90%. In another embodiment, the mean liver enzyme(s) concentration(s) in serum is reduced by at least about 95%. In another embodiment, the mean liver enzyme(s) concentration(s) in serum is reduced by at least about 100%. In some embodiments, the liver enzyme is selected from the group consisting of Alanine Transaminase (ALT) enzyme and Aspartate Transaminase (AST) enzyme. In some embodiments, the liver enzyme is ALT. In some embodiments, the liver enzyme is AST.

In some embodiments, the post-treatment pro-inflammatory cytokine(s) in serum is reduced to at least about one standard deviation lower than a pre-treatment level. In some embodiments, the post-treatment pro-inflammatory cytokine(s) in serum is reduced to at least about two standard deviation lower than a pre-treatment level. In some embodiments, the post-treatment pro-inflammatory cytokine(s) in serum is reduced to at least about three standard deviation lower than a pre-treatment level. In some embodiments, the pro-inflammatory cytokine(s) are reduced by at least about 10%, 15%, 20%, 25%, 30%, 35%, 40%, 45%, 50%, 55%, 60%, 65%, 70%, 75%, 80%, 85%, 90%, 95%, or 100%. In one embodiment, the pro-inflammatory cytokine(s) is reduced by at least about 10%. In another embodiment, the pro-inflammatory cytokine(s) is reduced by at least about 15%. In another embodiment, the pro-inflammatory cytokine(s) is reduced by at least about 20%. In another embodiment, the pro-inflammatory cytokine(s) is reduced by at least about 25%. In another embodiment, the pro-inflammatory cytokine(s) is reduced by at least about 30%. In another embodiment, the pro-inflammatory cytokine(s) is reduced by at least about 35%. In another embodiment, the pro-inflammatory cytokine(s) is reduced by at least about 40%. In another embodiment, the pro-inflammatory cytokine(s) is reduced by at least about 45%. In another embodiment, the pro-inflammatory cytokine(s) is reduced by at least about 50%. In another embodiment, the pro-inflammatory cytokine(s) is reduced by at least about 55%. In another embodiment, the pro-inflammatory cytokine(s) is reduced by at least about 60%. In another embodiment, the pro-inflammatory cytokine(s) is reduced by at least about 65%. In another embodiment, the pro-inflammatory cytokine(s) is reduced by at least about 70%. In another embodiment, the pro-inflammatory cytokine(s) is reduced by at least about 75%. In another embodiment, the pro-inflammatory cytokine(s) is reduced by at least about 80%. In another embodiment, the pro-inflammatory cytokine(s) is reduced by at least about 85%. In another embodiment, the pro-inflammatory cytokine(s) is reduced by at least about 90%. In another embodiment, the pro-inflammatory cytokine(s) is reduced by at least about 95%. In another embodiment, the pro-inflammatory cytokine(s) is reduced by at least about 100%. In some embodiments, the pro-inflammatory cytokine(s) is selected from the group consisting of IL-6, IL-8, MCP-1, and TNFₐ. In some embodiments, the pro-inflammatory cytokine(s) is selected from the group consisting of IL-6, IL-8, and TNFₐ. In some embodiments, the pro-inflammatory cytokine(s) is selected from the group consisting of IL-6, IL-8, and MCP-1. In some embodiments, the pro-inflammatory cytokine(s) is IL-6. In some embodiments, the pro-inflammatory cytokine(s) is IL-8. In some embodiments, the pro-inflammatory cytokine(s) is MCP-1. In some embodiments, the pro-inflammatory cytokine(s) is TNFₐ. Herein, TNFₐ is used interchangeably with TNF-α and TNF-alpha.

In some embodiments, the disclosure embraces a method of mitigating inflammation and/or oxidative stress in a biological system comprising:

administering one or more compounds to the biological system experiencing inflammation or oxidative stress, the one or more compounds comprising:

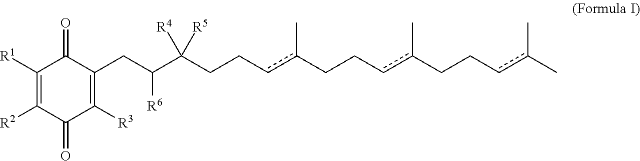

wherein:
each bond indicated with a dashed line is independently a single bond or a double bond;
R¹, R², and R³ are independently selected from H, (C₁-C₄)-alkyl, (C₁-C₄)-haloalkyl, —CN, —F, —Cl, —Br, and —I; and
R⁴ and R⁵ are independently selected from hydroxy and (C₁-C₄)-alkyl, and R⁶ is hydrogen; or
R⁴ is (C₁-C₄)-alkyl, and R⁵ and R⁶ are hydrogen; or
R⁴ is (C₁-C₄)-alkyl, and R⁵ and R⁶ together form the second bond of a double bond between the carbon atoms to which they are attached; or a stereoisomer, mixtures of stereoisomers, hydroquinone form, prodrug, metabolite, salt, phosphate substituted form, crystalline form, non-crystalline form, isotopologue, deuterated form, hydrate, or solvate thereof.

In some embodiments, the one or more compounds is selected from the group consisting of alpha-tocotrienol quinone, beta-tocotrienol quinone, gamma-tocotrienol quinone, delta-tocotrienol quinone, alpha-tocopherol quinone, beta-tocopherol quinone, gamma-tocopherol quinone, delta-tocopherol quinone, and stereoisomers, mixtures of stereoisomers, hydrates, and solvates thereof.

In some embodiments, the one or more compounds is selected from the group consisting of alpha-tocotrienol quinone, beta-tocotrienol quinone, gamma-tocotrienol quinone, and delta-tocotrienol quinone, and stereoisomers, mixtures of stereoisomers, hydrates, and solvates thereof.

In some embodiments, the one or more compounds is selected from the group consisting of alpha-tocopherol quinone, beta- tocopherol quinone, gamma-tocopherol quinone, and delta- tocopherol quinone, and stereoisomers, mixtures of stereoisomers, hydrates, and solvates thereof.

In some embodiments, the one or more compounds is selected from the group consisting of alpha-tocotrienol quinone, and stereoisomers, mixtures of stereoisomers, hydrates, and solvates thereof.

In some embodiments, the one or more compounds is selected from the group consisting of alpha-tocopherol quinone, and stereoisomers, mixtures of stereoisomers, hydrates, and solvates thereof.

In some embodiments, the administering step of the one or more compounds is done orally, via a pharmaceutical formulation, medical food, or an ingestible supplement. In alternative embodiments, the one or more compounds can be administered via injection or topically.

In some embodiments, administering is done in conjunction with a member of the group consisting of NAC or DHA. In some embodiments, this member is present in a weight % concentration of 1-99% of the one or more compounds.

In some embodiments, the biological system is a eukaryotic system, and more specifically can be a human subject. In some embodiments, the human is an adult. In some embodiments, the human is an adult eighteen-years old or older. In some embodiments, the human is an adult male. In some embodiments, the human is an adult female. In some embodiments, the human is a child under the age of eighteen-years old. In some embodiments, the child is a child male. In some embodiments, the human is a child female. The administering step can comprise 100 mg to 500 mg of the one or more compounds per kg of body weight; alternatively 100 mg to 300 mg of the one or more compounds per kg of body weight, or alternatively 100 mg to 200 mg of the one or more compounds per kg of body weight.

In some embodiments, the subject is selected from the group consisting of a patient experiencing one or more inflammation and/or oxidative stress symptoms associated with a liver, kidney, eye, skin, lung, heart, vascular, bone marrow, immune function, CNS, or musculoskeletal disease state. In some embodiments, the subject is selected from the group consisting of a patient experiencing one or more inflammation and/or oxidative stress symptoms associated with a liver, kidney, eye, or skin disease states. In some embodiments, the disease states associated with the liver are selected from the group consisting of NASH/NAFL, alcoholic hepatitis, cholestatic liver disease, viral hepatitis, drug-induced liver toxicity, hemachromatosis, Wilson's disease, transplant reperfusion injury, and hepatic insufficiency where the hepatic insufficiency is due to, in some nonlimiting embodiments, injury (including but not limited to injury to the liver), SIRS, sepsis, and severe illness.

In some embodiments, additional disease states characteristic of inflammation and/or oxidative stress are selected from the group consisting of cisplatin-induced liver toxicity, cisplatin-induced kidney toxicity, diabetic nephropathy, acute tubular necrosis, ICU-related cognitive dysfunction, haptoglobin deficient metabolic syndrome, end stage renal disease, sarcopenia, non-alcoholic fatty liver disease (NAFLD), pediatric NAFLD, age-related conditions, e.g., age-related eye disorders (AREDS), age-related macular degeneration, and age-related muscle loss (sarcopenia), and skin disorders, e.g., rosacea, eczema, acne, psoriasis, wound healing, scarring, and radiation damage, including acute skin damage caused by radiation therapy as well as transient radiation induced inflammation. In some embodiments, the compositions herein may be used to retard the progression of aging-related conditions, such as age-related skin conditions such as fine lines and wrinkles and decreased tone and texture.

In some or any embodiments disclosed herein the compound is not a prodrug, metabolite, salt, phosphate substituted form, crystalline form, non-crystalline form, isotopologue, deuterated form, hydrate, or solvate thereof. In some or any embodiments disclosed herein the compound is a salt, a stereoisomer, a mixture of stereoisomers, a hydrate, or solvate thereof. In some or any embodiments disclosed herein the compound is a stereoisomer or a mixture of stereoisomers thereof.

In some embodiments of the disclosure, including any of the foregoing embodiments, the one or more compounds comprises one or more compounds selected from the group consisting of alpha-tocotrienol quinone, beta-tocotrienol quinone, gamma-tocotrienol quinone, and delta-tocotrienol quinone. In some embodiments, the one or more compounds comprises alpha-tocotrienol quinone. In some embodiments, the one or more compounds comprises beta-tocotrienol quinone. In some embodiments, the one or more compounds comprises gamma-tocotrienol quinone. In some embodiments, the one or more compounds comprises delta-tocotrienol quinone.

In some embodiments of the disclosure, including any of the foregoing embodiments, the one or more compounds comprises one or more compounds selected from the group consisting of alpha-tocopherol quinone, beta-tocopherol quinone, gamma-tocopherol quinone, and delta-tocopherol quinone. In some embodiments, the one or more compounds comprises alpha-tocopherol quinone. In some embodiments, the one or more compounds comprises beta-tocopherol quinone. In some embodiments, the one or more compounds comprises gamma-tocopherol quinone. In some embodiments, the one or more compounds comprises delta-tocopherol quinone.

In some embodiments, the one or more compounds are used therapeutically during, after, or during and after exposure to a factor which may result in inflammation and/or oxidative stress, by administering a therapeutically effective amount to a subject in need thereof. In some embodiments, the one or more compounds are used prophylactically prior to exposure to a factor which may result in inflammation and/or oxidative stress, by administering a prophylactically effective amount to a subject in need (or potential need) thereof.

The compounds described herein may also be used in a method of increasing the therapeutic window of a drug that has potentially toxic effects due to its ability to cause inflammation and/or oxidative stress in a tissue. For example, certain drugs, such as acetaminophen, can be toxic for the liver above certain doses. By co-administering one or more compounds described herein in conjunction with the drug, either in the same composition or as separate compositions, the drug may safely be administered at higher doses.

Any one or more of the compounds described herein, including all of the foregoing compounds, can be used in a composition comprising a pharmaceutically acceptable carrier, pharmaceutically acceptable excipient, or pharmaceutically acceptable vehicle. Any one or more of the compounds described herein, including all of the foregoing compounds, can be formulated into a unit dose formulation.

In some or any embodiments, as disclosed herein, provided is a composition for use or a method of treatment of inflammation.

In some or any embodiments, as disclosed herein, provided is a composition for use or a method of treatment of oxidative stress.

In some or any embodiments, as disclosed herein, provided is a composition for use or a method of treatment wherein the inflammation and/or oxidative stress is not associated with an increase in a thiosulfate level, a decrease in a hydrogen sulfide level, and/or associated with preventing an increased hydrogen sulfide level in a subject. In some or any embodiments, as disclosed herein, provided is a composition for use or a method of treatment wherein the inflammation and/or oxidative stress is not associated with an increase in a thiosulfate level, a decrease in a hydrogen sulfide level, and/or associated with preventing an increased hydrogen sulfide level in a subject, when the inflammation and/or oxidative stress is selected from diabetes, liver damage, insulin resistance, nephropathy, acute or chronic renal injury due to exposure to nephrotoxic agents, drug induced peripheral neuropathy, serious atopic dermatitis, psoriasis, eczema, cyanide poisoning, a mitochondrial disorder; an inherited mitochondrial disease; Alpers Disease; Barth syndrome; a Beta-oxidation Defect; Carnitine-Acyl-Carnitine Deficiency; Carnitine Deficiency; a Creatine Deficiency Syndrome; Co-Enzyme Q10 Deficiency; Complex I Deficiency; Complex II Deficiency; Complex III Deficiency; Complex IV Deficiency; Complex V Deficiency; COX Deficiency; chronic progressive external ophthalmoplegia (CPEO); CPT I Deficiency; CPT II Deficiency; Friedreich's Ataxia (FA); Glutaric Aciduria Type II; Kearns-Sayre Syndrome (KSS); Lactic Acidosis; Long-Chain Acyl-CoA Dehydrongenase Deficiency (LCAD); LCHAD; Leigh Syndrome; Leigh-like Syndrome; Leber's Hereditary Optic Neuropathy (LHON); Lethal Infantile Cardiomyopathy (LIC); Luft Disease; Multiple Acyl-CoA Dehydrogenase Deficiency (MAD); Medium-Chain Acyl-CoA Dehydrongenase Deficiency (MCAD); Mitochondrial Myopathy, Encephalopathy, Lactacidosis, Stroke (MELAS); Myoclonic Epilepsy with Ragged Red Fibers (MERRF); Mitochondrial Recessive Ataxia Syndrome (MIRAS); Mitochondrial Cytopathy; Mitochondrial DNA Depletion; Mitochondrial Encephalopathy; Mitochondrial Myopathy; Myoneurogastointestinal Disorder and Encephalopathy (MNGIE); Neuropathy, Ataxia, and Retinitis Pigmentosa (NARP); Pearson Syndrome; Pyruvate Carboxylase Deficiency; Pyruvate Dehydrogenase Deficiency; a POLG Mutation; a Respiratory Chain Disorder; Short-Chain Acyl-CoA Dehydrogenase Deficiency (SCAD); SCHAD; Very Long-Chain Acyl-CoA Dehydrongenase Deficiency (VLCAD); a myopathy; cardiomyopathy; encephalomyopathy; a neurodegenerative disease; Parkinson's disease; Alzheimer's disease; amyotrophic lateral sclerosis (ALS); a motor neuron disease; a neurological disease; epilepsy; an age-associated disease; macular degeneration; diabetes; metabolic syndrome; cancer; brain cancer; a genetic disease; Huntington's Disease; a mood disorder; schizophrenia; bipolar disorder; a pervasive developmental disorder; autistic disorder; Asperger's syndrome; childhood disintegrative disorder (CDD); Rett's disorder; PDD-not otherwise specified (PDD-NOS); a cerebrovascular accident; stroke; a vision impairment; optic neuropathy; dominant inherited juvenile optic atrophy; optic neuropathy caused by a toxic agent; glaucoma; Stargardt's macular dystrophy; diabetic retinopathy; diabetic maculopathy; retinopathy of prematurity; ischemic reperfusion-related retinal injury; oxygen poisoning; a haemoglobionopathy; thalassemia; sickle cell anemia; seizures; ischemia; renal tubular acidosis; attention deficit/hyperactivity disorder (ADHD); a neurodegenerative disorder resulting in hearing or balance impairment; Dominant Optic Atrophy (DOA); Maternally inherited diabetes and deafness (MIDD); chronic fatigue; contrast-induced kidney damage; contrast-induced retinopathy damage; Abetalipoproteinemia; retinitis pigmentosum; Wolfram's disease; Tourette syndrome; cobalamin c defect; methylmalonic aciduria; glioblastoma; Down's syndrome; acute tubular necrosis; a muscular dystrophy; a leukodystrophy; Progressive Supranuclear Palsy; spinal muscular atrophy; hearing loss; noise induced hearing loss; traumatic brain injury; Juvenile Huntington's Disease; Multiple Sclerosis; NGLY1; Multisystem atrophy; Adrenoleukodystrophy; and Adrenomyeloneuropathy, decreased renal function, acute lung injury, calciphylaxis in blood vessels, kidney toxicity in cancer therapy, bacterial infection, fungal infection, ulcerative colitis, inflammatory bowel disease, hypertension, proteinuria, ischemia, hypoxia, atherosclerosis, uterus obstruction, hyperalgesia, lactic acidosis, Kearns-Sayre Syndrome (KSS), and primary progressive multiple sclerosis. In some or any embodiments, as disclosed herein, provided is a composition for use or a method of treatment wherein the inflammation and/or oxidative stress is not associated with an increase in a thiosulfate level, a decrease in a hydrogen sulfide level, and/or associated with preventing an increased hydrogen sulfide level in a subject, when the inflammation and/or oxidative stress is selected from diabetes, liver damage, insulin resistance, nephropathy, acute or chronic renal injury due to exposure to nephrotoxic agents, drug induced peripheral neuropathy, serious atopic dermatitis, psoriasis, eczema, decreased renal function, kidney toxicity in cancer therapy, ulcerative colitis, and inflammatory bowel disease, diabetes, liver damage, insulin resistance, nephropathy, acute or chronic renal injury due to exposure to nephrotoxic agents, drug induced peripheral neuropathy, serious atopic dermatitis, psoriasis, eczema, cyanide poisoning, a mitochondrial disorder; an inherited mitochondrial disease; Alpers Disease; Barth syndrome; a Beta-oxidation Defect; Carnitine-Acyl-Carnitine Deficiency; Carnitine Deficiency; a Creatine Deficiency Syndrome; Co-Enzyme Q10 Deficiency; Complex I Deficiency; Complex II Deficiency; Complex III Deficiency; Complex IV Deficiency; Complex V Deficiency; COX Deficiency; chronic progressive external ophthalmoplegia (CPEO); CPT I Deficiency; CPT II Deficiency; Friedreich's Ataxia (FA); Glutaric Aciduria Type II; Kearns-Sayre Syndrome (KSS); Lactic Acidosis; Long-Chain Acyl-CoA Dehydrongenase Deficiency (LCAD); LCHAD; Leigh Syndrome; Leigh-like Syndrome; Leber's Hereditary Optic Neuropathy (LHON); Lethal Infantile Cardiomyopathy (LIC); Luft Disease; Multiple Acyl-CoA Dehydrogenase Deficiency (MAD); Medium-Chain Acyl-CoA Dehydrongenase Deficiency (MCAD); Mitochondrial Myopathy, Encephalopathy, Lactacidosis, Stroke (MELAS); Myoclonic Epilepsy with Ragged Red Fibers (MERRF); Mitochondrial Recessive Ataxia Syndrome (MIRAS); Mitochondrial Cytopathy; Mitochondrial DNA Depletion; Mitochondrial Encephalopathy; Mitochondrial Myopathy; Myoneurogastointestinal Disorder and Encephalopathy (MNGIE); Neuropathy, Ataxia, and Retinitis Pigmentosa (NARP); Pearson Syndrome; Pyruvate Carboxylase Deficiency; Pyruvate Dehydrogenase Deficiency; a POLG Mutation; a Respiratory Chain Disorder; Short-Chain Acyl-CoA Dehydrogenase Deficiency (SCAD); SCHAD; Very Long-Chain Acyl-CoA Dehydrongenase Deficiency (VLCAD); a myopathy; cardiomyopathy; encephalomyopathy; a neurodegenerative disease; Parkinson's disease; Alzheimer's disease; amyotrophic lateral sclerosis (ALS); a motor neuron disease; a neurological disease; epilepsy; an age-associated disease; macular degeneration; diabetes; metabolic syndrome; cancer; brain cancer; a genetic disease; Huntington's Disease; a mood disorder; schizophrenia; bipolar disorder; a pervasive developmental disorder; autistic disorder; Asperger's syndrome; childhood disintegrative disorder (CDD); Rett's disorder; PDD-not otherwise specified (PDD-NOS); a cerebrovascular accident; stroke; a vision impairment; optic neuropathy; dominant inherited juvenile optic atrophy; optic neuropathy caused by a toxic agent; glaucoma; Stargardt's macular dystrophy; diabetic retinopathy; diabetic maculopathy; retinopathy of prematurity; ischemic reperfusion-related retinal injury; oxygen poisoning; a haemoglobionopathy; thalassemia; sickle cell anemia; seizures; ischemia; renal tubular acidosis; attention deficit/hyperactivity disorder (ADHD); a neurodegenerative disorder resulting in hearing or balance impairment; Dominant Optic Atrophy (DOA); Maternally inherited diabetes and deafness (MIDD); chronic fatigue; contrast-induced kidney damage; contrast-induced retinopathy damage; Abetalipoproteinemia; retinitis pigmentosum; Wolfram's disease; Tourette syndrome; cobalamin c defect; methylmalonic aciduria; glioblastoma; Down's syndrome; acute tubular necrosis; a muscular dystrophy; a leukodystrophy; Progressive Supranuclear Palsy; spinal muscular atrophy; hearing loss; noise induced hearing loss; traumatic brain injury; Juvenile Huntington's Disease; Multiple Sclerosis; NGLY1; Multisystem atrophy; Adrenoleukodystrophy; and Adrenomyeloneuropathy, decreased renal function, acute lung injury, calciphylaxis in blood vessels, kidney toxicity in cancer therapy, bacterial infection, fungal infection, ulcerative colitis, inflammatory bowel disease, hypertension, proteinuria, ischemia, hypoxia, atherosclerosis, uterus obstruction, hyperalgesia, lactic acidosis, Kearns-Sayre Syndrome (KSS), and primary progressive multiple sclerosis.

In some or any embodiments, as disclosed herein, provided is a composition for use or a method of treatment wherein the inflammation and/or oxidative stress is not associated with an increase in a thiosulfate level. In some or any embodiments, as disclosed herein, provided is a composition for use or a method of treatment wherein the inflammation and/or oxidative stress is not associated with an increase in a thiosulfate level when the inflammation and/or oxidative stress is selected from cyanide poisoning, decreased renal function, acute lung injury, calciphylaxis in blood vessels, kidney toxicity in cancer therapy, bacterial infection, fungal infection, ulcerative colitis, hypertension, proteinuria, ischemia, hypoxia, atherosclerosis, uterus obstruction, diabetes, liver damage, insulin resistance, nephropathy, acute or chronic renal injury due to exposure to nephrotoxic agents, drug induced peripheral neuropathy, hyperalgesia, inflammatory bowels disease, lactic acidosis, Kearns-Sayre Syndrome (KSS), primary progressive multiple sclerosis, serious atopic dermatitis, psoriasis, and eczema. In some or any embodiments, as disclosed herein, provided is a composition for use or a method of treatment wherein the inflammation and/or oxidative stress is not associated with an increase in a thiosulfate level when the inflammation and/or oxidative stress is selected from liver damage, insulin resistance, nephropathy, acute or chronic renal injury due to exposure to nephrotoxic agents, drug induced peripheral neuropathy, serious atopic dermatitis, psoriasis, and eczema.

In some or any embodiments, as disclosed herein, provided is a composition for use or a method of treatment wherein the inflammation and/or oxidative stress is not associated with a decrease in a hydrogen sulfide level in a subject. In some or any embodiments, as disclosed herein, provided is a composition for use or a method of treatment wherein the inflammation and/or oxidative stress is not associated with a decrease in a hydrogen sulfide level in a subject, when the inflammation and/or oxidative stress is selected from diabetes, liver damage, insulin resistance, nephropathy, acute or chronic renal injury due to exposure to nephrotoxic agents, drug induced peripheral neuropathy, serious atopic dermatitis, psoriasis, eczema, cyanide poisoning, decreased renal function, acute lung injury, calciphylaxis in blood vessels, kidney toxicity in cancer therapy, bacterial infection, fungal infection, ulcerative colitis, inflammatory bowel disease, hypertension, proteinuria, ischemia, hypoxia, atherosclerosis, uterus obstruction, hyperalgesia, lactic acidosis, Kearns-Sayre Syndrome (KSS), and primary progressive multiple sclerosis. In some or any embodiments, as disclosed herein, provided is a composition for use or a method of treatment wherein the inflammation and/or oxidative stress is not associated with a decrease in a hydrogen sulfide level in a subject, when the inflammation and/or oxidative stress is selected from diabetes, liver damage, insulin resistance, nephropathy, acute or chronic renal injury due to exposure to nephrotoxic agents, drug induced peripheral neuropathy, serious atopic dermatitis, psoriasis, eczema, decreased renal function, kidney toxicity in cancer therapy, ulcerative colitis, and inflammatory bowel disease.

In some or any embodiments, as disclosed herein, provided is a composition for use or a method of treatment wherein the inflammation and/or oxidative stress is not associated with preventing an increased hydrogen sulfide level in a subject. In some or any embodiments, as disclosed herein, provided is a composition for use or a method of treatment wherein the inflammation and/or oxidative stress is not associated with preventing an increased hydrogen sulfide level in a subject, when the inflammation and/or oxidative stress is selected from diabetes, liver damage, insulin resistance, nephropathy, acute or chronic renal injury due to exposure to nephrotoxic agents, drug induced peripheral neuropathy, serious atopic dermatitis, psoriasis, eczema, cyanide poisoning, decreased renal function, acute lung injury, calciphylaxis in blood vessels, kidney toxicity in cancer therapy, bacterial infection, fungal infection, ulcerative colitis, inflammatory bowel disease, hypertension, proteinuria, ischemia, hypoxia, atherosclerosis, uterus obstruction, hyperalgesia, lactic acidosis, Kearns-Sayre Syndrome (KSS), and primary progressive multiple sclerosis. In some or any embodiments, as disclosed herein, provided is a composition for use or a method of treatment wherein the inflammation and/or oxidative stress is not associated with preventing an increased hydrogen sulfide level in a subject, when the inflammation and/or oxidative stress is selected from diabetes, liver damage, insulin resistance, nephropathy, acute or chronic renal injury due to exposure to nephrotoxic agents, drug induced peripheral neuropathy, serious atopic dermatitis, psoriasis, eczema, decreased renal function, kidney toxicity in cancer therapy, ulcerative colitis, and inflammatory bowel disease.

In some or any embodiments, as disclosed herein, provided is a composition for use or a method of treatment wherein the inflammation and/or oxidative stress is selected from diabetes, liver damage, insulin resistance, nephropathy, acute or chronic renal injury due to exposure to nephrotoxic agents, drug induced peripheral neuropathy, serious atopic dermatitis, psoriasis, eczema, cyanide poisoning, decreased renal function, acute lung injury, calciphylaxis in blood vessels, kidney toxicity in cancer therapy, bacterial infection, fungal infection, ulcerative colitis, inflammatory bowel disease, hypertension, proteinuria, ischemia, hypoxia, atherosclerosis, uterus obstruction, hyperalgesia, lactic acidosis, Kearns-Sayre Syndrome (KSS), and primary progressive multiple sclerosis; and wherein the inflammation and/or oxidative stress is not associated with an increase in a thiosulfate level, a decrease in a hydrogen sulfide level, and/or associated with preventing an increased hydrogen sulfide level in a subject. In some or any embodiments, as disclosed herein, provided is a composition for use or a method of treatment wherein the inflammation and/or oxidative stress is selected from diabetes, liver damage, insulin resistance, nephropathy, acute or chronic renal injury due to exposure to nephrotoxic agents, drug induced peripheral neuropathy, serious atopic dermatitis, psoriasis, eczema, decreased renal function, kidney toxicity in cancer therapy, ulcerative colitis, and inflammatory bowel disease; and wherein the inflammation and/or oxidative stress is not associated with an increase in a thiosulfate level, a decrease in a hydrogen sulfide level, and/or associated with preventing an increased hydrogen sulfide level in a subject.

In some or any embodiments, as disclosed herein, provided is a composition for use or a method of treatment wherein the inflammation and/or oxidative stress is selected from cyanide poisoning, decreased renal function, acute lung injury, calciphylaxis in blood vessels, kidney toxicity in cancer therapy, bacterial infection, fungal infection, ulcerative colitis, hypertension, proteinuria, ischemia, hypoxia, atherosclerosis, uterus obstruction, diabetes, liver damage, insulin resistance, nephropathy, acute or chronic renal injury due to exposure to nephrotoxic agents, drug induced peripheral neuropathy, hyperalgesia, inflammatory bowels disease, lactic acidosis, Kearns-Sayre Syndrome (KSS), primary progressive multiple sclerosis, serious atopic dermatitis, psoriasis, and eczema; and wherein the inflammation and/or oxidative stress is not associated with an increase in a thiosulfate level. In some or any embodiments, as disclosed herein, provided is a composition for use or a method of treatment wherein the inflammation and/or oxidative stress is selected from liver damage, insulin resistance, nephropathy, acute or chronic renal injury due to exposure to nephrotoxic agents, drug induced peripheral neuropathy, serious atopic dermatitis, psoriasis, and eczema; and wherein the inflammation and/or oxidative stress is not associated with an increase in a thiosulfate level.

In some or any embodiments, as disclosed herein, provided is a composition for use or a method of treatment wherein the inflammation and/or oxidative stress is selected from diabetes, liver damage, insulin resistance, nephropathy, acute or chronic renal injury due to exposure to nephrotoxic agents, drug induced peripheral neuropathy, serious atopic dermatitis, psoriasis, eczema, cyanide poisoning, decreased renal function, acute lung injury, calciphylaxis in blood vessels, kidney toxicity in cancer therapy, bacterial infection, fungal infection, ulcerative colitis, inflammatory bowel disease, hypertension, proteinuria, ischemia, hypoxia, atherosclerosis, uterus obstruction, hyperalgesia, lactic acidosis, Kearns-Sayre Syndrome (KSS), and primary progressive multiple sclerosis; and wherein the inflammation and/or oxidative stress is not associated with a decrease in a hydrogen sulfide level in a subject. In some or any embodiments, as disclosed herein, provided is a composition for use or a method of treatment wherein the inflammation and/or oxidative stress is selected from diabetes, liver damage, insulin resistance, nephropathy, acute or chronic renal injury due to exposure to nephrotoxic agents, drug induced peripheral neuropathy, serious atopic dermatitis, psoriasis, eczema, decreased renal function, kidney toxicity in cancer therapy, ulcerative colitis, and inflammatory bowel disease; and wherein the inflammation and/or oxidative stress is not associated with a decrease in a hydrogen sulfide level in a subject.

In some or any embodiments, as disclosed herein, provided is a composition for use or a method of treatment wherein the inflammation and/or oxidative stress is selected from diabetes, liver damage, insulin resistance, nephropathy, acute or chronic renal injury due to exposure to nephrotoxic agents, drug induced peripheral neuropathy, serious atopic dermatitis, psoriasis, eczema, cyanide poisoning, decreased renal function, acute lung injury, calciphylaxis in blood vessels, kidney toxicity in cancer therapy, bacterial infection, fungal infection, ulcerative colitis, inflammatory bowel disease, hypertension, proteinuria, ischemia, hypoxia, atherosclerosis, uterus obstruction, hyperalgesia, lactic acidosis, Kearns-Sayre Syndrome (KSS), and primary progressive multiple sclerosis; and wherein the inflammation and/or oxidative stress is not associated with preventing an increased hydrogen sulfide level in a subject. In some or any embodiments, as disclosed herein, provided is a composition for use or a method of treatment wherein the inflammation and/or oxidative stress is selected from diabetes, liver damage, insulin resistance, nephropathy, acute or chronic renal injury due to exposure to nephrotoxic agents, drug induced peripheral neuropathy, serious atopic dermatitis, psoriasis, eczema, decreased renal function, kidney toxicity in cancer therapy, ulcerative colitis, and inflammatory bowel disease; and wherein the inflammation and/or oxidative stress is not associated with preventing an increased hydrogen sulfide level in a subject.

In some or any embodiments, as disclosed herein, provided is a composition for use or a method of treatment wherein the inflammation and/or oxidative stress is serious atopic dermatitis, and wherein the serious atopic dermatitis is not associated with a need to increase a thiosulfate level, a decrease in a hydrogen sulfide level, and/or associated with preventing an increased hydrogen sulfide level in a subject. In some or any embodiments, as disclosed herein, provided is a composition for use or a method of treatment wherein the inflammation and/or oxidative stress is psoriasis, and wherein the psoriasis is not associated with a need to increase a thiosulfate level. In some or any embodiments, as disclosed herein, provided is a composition for use or a method of treatment wherein the inflammation and/or oxidative stress is eczema, and wherein the eczema is not associated with a need to increase a thiosulfate level, a decrease in a hydrogen sulfide level, and/or associated with preventing an increased hydrogen sulfide level in a subject. In some or any embodiments, as disclosed herein, provided is a composition for use or a method of treatment wherein the inflammation and/or oxidative stress is serious atopic dermatitis, and wherein the serious atopic dermatitis is not associated with a need to increase a thiosulfate level. In some or any embodiments, as disclosed herein, provided is a composition for use or a method of treatment wherein the inflammation and/or oxidative stress is psoriasis, and wherein the psoriasis is not associated with a need to increase a thiosulfate level. In some or any embodiments, as disclosed herein, provided is a composition for use or a method of treatment wherein the inflammation and/or oxidative stress is eczema, and wherein the eczema is not associated with a need to increase a thiosulfate level.

In some embodiments of the present disclosure, the one or more compounds is selected from the group consisting of alpha-tocotrienol quinone, and stereoisomers, mixtures of stereoisomers, hydrates, and solvates thereof and the inflammation or oxidative stress is associated with psoriasis. In some embodiments of the present disclosure, the one or more compounds is selected from the group consisting of alpha-tocotrienol quinone, and stereoisomers, mixtures of stereoisomers, hydrates, and solvates thereof and the inflammation or oxidative stress is associated with eczema. In some embodiments of the present disclosure, the one or more compounds is selected from the group consisting of alpha-tocotrienol quinone, and stereoisomers, mixtures of stereoisomers, hydrates, and solvates thereof and the inflammation or oxidative stress is associated with serious atopic dermatitis. In some embodiments of the present disclosure, the one or more compounds is selected from the group consisting of alpha-tocotrienol quinone, and stereoisomers, mixtures of stereoisomers, hydrates, and solvates thereof and the inflammation or oxidative stress is associated with NASH/NAFL. In some embodiments of the present disclosure, the one or more compounds is selected from the group consisting of alpha-tocotrienol quinone, and stereoisomers, mixtures of stereoisomers, hydrates, and solvates thereof and the inflammation or oxidative stress is associated with alcoholic hepatitis. In some embodiments of the present disclosure, the one or more compounds is selected from the group consisting of alpha-tocotrienol quinone, and stereoisomers, mixtures of stereoisomers, hydrates, and solvates thereof and the inflammation or oxidative stress is associated with cholestatic liver disease. In some embodiments of the present disclosure, the one or more compounds is selected from the group consisting of alpha-tocotrienol quinone, and stereoisomers, mixtures of stereoisomers, hydrates, and solvates thereof and the inflammation or oxidative stress is associated with viral hepatitis. In some embodiments of the present disclosure, the one or more compounds is selected from the group consisting of alpha-tocotrienol quinone, and stereoisomers, mixtures of stereoisomers, hydrates, and solvates thereof and the inflammation or oxidative stress is associated with drug-induced liver toxicity. In some embodiments of the present disclosure, the one or more compounds is selected from the group consisting of alpha-tocotrienol quinone, and stereoisomers, mixtures of stereoisomers, hydrates, and solvates thereof and the inflammation or oxidative stress is associated with hemochromatosis. In some embodiments of the present disclosure, the one or more compounds is selected from the group consisting of alpha-tocotrienol quinone, and stereoisomers, mixtures of stereoisomers, hydrates, and solvates thereof and the inflammation or oxidative stress is associated with Wilson's disease. In some embodiments of the present disclosure, the one or more compounds is selected from the group consisting of alpha-tocotrienol quinone, and stereoisomers, mixtures of stereoisomers, hydrates, and solvates thereof and the inflammation or oxidative stress is associated with transplant reperfusion injury. In some embodiments of the present disclosure, the one or more compounds is selected from the group consisting of alpha-tocotrienol quinone, and stereoisomers, mixtures of stereoisomers, hydrates, and solvates thereof and the inflammation or oxidative stress is associated with hepatic insufficiency where the hepatic insufficiency is due to injury, SIRS, sepsis, and/or severe illness. In some embodiments of the present disclosure, the one or more compounds is selected from the group consisting of alpha-tocotrienol quinone, and stereoisomers, mixtures of stereoisomers, hydrates, and solvates thereof and the inflammation or oxidative stress is associated with drug-induced liver toxicity. In some embodiments of the present disclosure, the one or more compounds is selected from the group consisting of alpha-tocotrienol quinone, and stereoisomers, mixtures of stereoisomers, hydrates, and solvates thereof and the inflammation or oxidative stress is associated with a liver disease. In some embodiments of the present disclosure, the one or more compounds is selected from the group consisting of alpha-tocotrienol quinone, and stereoisomers, mixtures of stereoisomers, hydrates, and solvates thereof and the inflammation or oxidative stress is associated with kidney disease. In some embodiments of the present disclosure, the one or more compounds is selected from the group consisting of alpha-tocotrienol quinone, and stereoisomers, mixtures of stereoisomers, hydrates, and solvates thereof and the inflammation or oxidative stress is associated with eye disease. In some embodiments of the present disclosure, the one or more compounds is selected from the group consisting of alpha-tocotrienol quinone, and stereoisomers, mixtures of stereoisomers, hydrates, and solvates thereof and the inflammation or oxidative stress is associated with skin disease. In some embodiments of the present disclosure, the one or more compounds is selected from the group consisting of alpha-tocotrienol quinone, and stereoisomers, mixtures of stereoisomers, hydrates, and solvates thereof and the inflammation or oxidative stress is associated with diabetes. In some embodiments of the present disclosure, the one or more compounds is selected from the group consisting of alpha-tocotrienol quinone, and stereoisomers, mixtures of stereoisomers, hydrates, and solvates thereof and the inflammation or oxidative stress is associated with liver damage. In some embodiments of the present disclosure, the one or more compounds is selected from the group consisting of alpha-tocotrienol quinone, and stereoisomers, mixtures of stereoisomers, hydrates, and solvates thereof and the inflammation or oxidative stress is associated with insulin resistance. In some embodiments of the present disclosure, the one or more compounds is selected from the group consisting of alpha-tocotrienol quinone, and stereoisomers, mixtures of stereoisomers, hydrates, and solvates thereof and the inflammation or oxidative stress is associated with nephropathy. In some embodiments of the present disclosure, the one or more compounds is selected from the group consisting of alpha-tocotrienol quinone, and stereoisomers, mixtures of stereoisomers, hydrates, and solvates thereof and the inflammation or oxidative stress is associated with acute or chronic renal injury due to exposure to nephrotoxic agents. In some embodiments of the present disclosure, the one or more compounds is selected from the group consisting of alpha-tocotrienol quinone, and stereoisomers, mixtures of stereoisomers, hydrates, and solvates thereof and the inflammation or oxidative stress is associated with drug induced peripheral neuropathy. In some embodiments of the present disclosure, the one or more compounds is selected from the group consisting of alpha-tocotrienol quinone, and stereoisomers, mixtures of stereoisomers, hydrates, and solvates thereof and the inflammation or oxidative stress is associated with cyanide poisoning. In some embodiments of the present disclosure, the one or more compounds is selected from the group consisting of alpha-tocotrienol quinone, and stereoisomers, mixtures of stereoisomers, hydrates, and solvates thereof and the inflammation or oxidative stress is associated with decreased renal function. In some embodiments of the present disclosure, the one or more compounds is selected from the group consisting of alpha-tocotrienol quinone, and stereoisomers, mixtures of stereoisomers, hydrates, and solvates thereof and the inflammation or oxidative stress is associated with acute lung injury. In some embodiments of the present disclosure, the one or more compounds is selected from the group consisting of alpha-tocotrienol quinone, and stereoisomers, mixtures of stereoisomers, hydrates, and solvates thereof and the inflammation or oxidative stress is associated with calciphylaxis in blood vessels. In some embodiments of the present disclosure, the one or more compounds is selected from the group consisting of alpha-tocotrienol quinone, and stereoisomers, mixtures of stereoisomers, hydrates, and solvates thereof and the inflammation or oxidative stress is associated with kidney toxicity in cancer therapy. In some embodiments of the present disclosure, the one or more compounds is selected from the group consisting of alpha-tocotrienol quinone, and stereoisomers, mixtures of stereoisomers, hydrates, and solvates thereof and the inflammation or oxidative stress is associated with bacterial infection In some embodiments of the present disclosure, the one or more compounds is selected from the group consisting of alpha-tocotrienol quinone, and stereoisomers, mixtures of stereoisomers, hydrates, and solvates thereof and the inflammation or oxidative stress is associated with fungal infection. In some embodiments of the present disclosure, the one or more compounds is selected from the group consisting of alpha-tocotrienol quinone, and stereoisomers, mixtures of stereoisomers, hydrates, and solvates thereof and the inflammation or oxidative stress is associated with ulcerative colitis. In some embodiments of the present disclosure, the one or more compounds is selected from the group consisting of alpha-tocotrienol quinone, and stereoisomers, mixtures of stereoisomers, hydrates, and solvates thereof and the inflammation or oxidative stress is associated with inflammatory bowel disease. In some embodiments of the present disclosure, the one or more compounds is selected from the group consisting of alpha-tocotrienol quinone, and stereoisomers, mixtures of stereoisomers, hydrates, and solvates thereof and the inflammation or oxidative stress is associated with hypertension. In some embodiments of the present disclosure, the one or more compounds is selected from the group consisting of alpha-tocotrienol quinone, and stereoisomers, mixtures of stereoisomers, hydrates, and solvates thereof and the inflammation or oxidative stress is associated with proteinuria. In some embodiments of the present disclosure, the one or more compounds is selected from the group consisting of alpha-tocotrienol quinone, and stereoisomers, mixtures of stereoisomers, hydrates, and solvates thereof and the inflammation or oxidative stress is associated with ischemia. In some embodiments of the present disclosure, the one or more compounds is selected from the group consisting of alpha-tocotrienol quinone, and stereoisomers, mixtures of stereoisomers, hydrates, and solvates thereof and the inflammation or oxidative stress is associated with hypoxia. In some embodiments of the present disclosure, the one or more compounds is selected from the group consisting of alpha-tocotrienol quinone, and stereoisomers, mixtures of stereoisomers, hydrates, and solvates thereof and the inflammation or oxidative stress is associated with atherosclerosis. In some embodiments of the present disclosure, the one or more compounds is selected from the group consisting of alpha-tocotrienol quinone, and stereoisomers, mixtures of stereoisomers, hydrates, and solvates thereof and the inflammation or oxidative stress is associated with uterus obstruction. In some embodiments of the present disclosure, the one or more compounds is selected from the group consisting of alpha-tocotrienol quinone, and stereoisomers, mixtures of stereoisomers, hydrates, and solvates thereof and the inflammation or oxidative stress is associated with hyperalgesia. In some embodiments of the present disclosure, the one or more compounds is selected from the group consisting of alpha-tocotrienol quinone, and stereoisomers, mixtures of stereoisomers, hydrates, and solvates thereof and the inflammation or oxidative stress is associated with lactic acidosis. In some embodiments of the present disclosure, the one or more compounds is selected from the group consisting of alpha-tocotrienol quinone, and stereoisomers, mixtures of stereoisomers, hydrates, and solvates thereof and the inflammation or oxidative stress is associated with Kearns-Sayre Syndrome (KSS). In some embodiments of the present disclosure, the one or more compounds is selected from the group consisting of alpha-tocotrienol quinone, and stereoisomers, mixtures of stereoisomers, hydrates, and solvates thereof and the inflammation or oxidative stress is associated with primary progressive multiple sclerosis.

For all compounds, compositions, and formulations described herein, and all methods using a compound or composition or formulation described herein, the compounds or compositions can either comprise the listed components or steps, or can “consist essentially of” the listed components or steps, or can “consist of” the listed components or steps. That is, the transitional phrase “comprising” or “comprises” can be replaced by the transitional phrase “consisting essentially of” or “consists essentially of.” Alternatively, the transitional phrase “comprising” or “comprises” can be replaced, in some or any embodiments, by the transitional phrase “consisting of” or “consists of.” When a composition is described as “consisting essentially of” the listed components, the composition contains the components listed, and may contain other components which do not substantially affect the condition being treated, but do not contain any other components which substantially affect the condition being treated other than those components expressly listed; or, if the composition does contain extra components other than those listed which substantially affect the condition being treated, the composition does not contain a sufficient concentration or amount of the extra components to substantially affect the condition being treated. When a method is described as “consisting essentially of” the listed steps, the method contains the steps listed, and may contain other steps that do not substantially affect the condition being treated, but the method does not contain any other steps which substantially affect the condition being treated other than those steps expressly listed. As a non-limiting specific example, when a composition is described as ‘consisting essentially of’ a component, the composition may additionally contain any amount of pharmaceutically acceptable carriers, vehicles, excipients, or diluents and other such components which do not substantially affect the condition being treated.

In some embodiments, the inflammation or oxidative stress is not caused by a disorder described in the following applications, each of which is herein incorporated by reference in its entirety: PCT/US17/13271, PCT/US2017/13268, PCT/US06/21295, PCT/US09/035996, PCT/US09/056254, PCT/US09/060489, PCT/US10/62585, PCT/US10/32621, PCT/US10/32624, PCT/US10/46503, PCT/US12/045628, PCT/US12/047455, PCT/US2013/058568, PCT/US09/62212, PCT/US2015/063824, PCT/US11/31133, PCT/US12/045627, PCT/US12/036669, and PCT/US11/027690. In some embodiments, the inflammation or oxidative stress is not caused by a disorder described in the following application, which is herein incorporated by reference in its entirety: PCT/US12/041486, PCT/US17/13271, and PCT/US2017/13268.

The present disclosure provides compounds and compositions for use in treating or protecting against injury or damage caused by inflammation and/or oxidative stress, and methods of using such compounds for treating or for protecting against injury or damage caused by inflammation and/or oxidative stress.

The abbreviations used herein have their conventional meaning within the chemical and biological arts, unless otherwise specified.

The terms “a” or “an,” as used in herein means one or more, unless the context clearly dictates otherwise.

Reference to “about” a value or parameter herein includes (and describes) variations that are directed to that value or parameter per se. For example, description referring to “about X” includes description of “X”.

“Biological system” as used herein, and unless otherwise specified, refers to a eukaryotic organism, representative examples include animal and humans. A biological system includes “subjects”, as defined herein.

“Alkyl” is intended to embrace a saturated linear, branched, cyclic, or a combination thereof, hydrocarbon having the designated number of carbon atoms. For example, “C₁-C₆ alkyl” is intended to embrace a saturated linear, branched, cyclic, or a combination thereof, hydrocarbon of 1 to 6 carbon atoms. “C₁-C₄ alkyl” is intended to embrace a saturated linear, branched, cyclic, or a combination thereof, hydrocarbon of 1 to 4 carbon atoms. In some embodiments of “C₁-C₄ alkyl” are methyl, ethyl, n-propyl, isopropyl, cyclopropyl, n-butyl, isobutyl, sec-butyl, t-butyl, cyclobutyl, cyclopropyl-methyl, and methyl-cyclopropyl.

“Alkoxy” is intended to embrace —O-alkyl. For example, “C₁-C₆ alkoxy” is intended to embrace —O—C₁-C₆ alkyl.“Alkenyl” is intended to embrace a linear, branched, cyclic, or a combination thereof, hydrocarbon having the designated number of carbon atoms, and comprising one or more carbon-carbon double bonds.

“Haloalkoxy” is intended to embrace —O-haloalkyl, wherein haloalkyl is an alkyl group having the designated number of carbon atoms, and wherein the alkyl is substituted with one or more halo atoms.

“Carboxylic acid” is “C(O)OH”.

“DHA” means Docosohexanoic Acid.

“Drug induced liver injury (DILI)” may be caused by any drug that causes liver injury. In some embodiments, the drug causes inflammation in the liver. Non-limiting examples of drugs that cause DILI include acetaminophen and cisplatin.

“Halogen” or “halo” designates fluoro, chloro, bromo, and iodo.

“C₁-C₄ haloalkyl” is intended to embrace any C₁-C₄ alkyl substituent having at least one halogen substituent, in some embodiments 1 to 6 halogens, in some embodiments, 1 to 3 halogens; the halogen can be attached via any valence on the C₁-C₄ alkyl group. In some embodiments of C₁-C₄ haloalkyl is —CF₃, —CCl₃, —CHF₂, —CHCl₂, —CHBr₂, —CH₂F, —CH₂Cl.

“Deuterated form” means the compound is isotopically enriched for deuterium in at least one atom.

“Indicators” as used herein, and unless otherwise specified, refers to measureable biological variables representative of the status of biological function within a biological system.

“Isotopologue” means a compound which differs, i.e. in the number of neutrons, in its isotopic composition of at least one atom from the parent molecule having a natural isotopic composition. In some or any embodiments, the compound is isotopically enriched.

The term “isotopic composition,” as used herein, and unless otherwise specified, refers to the amount of each isotope present for a given atom, and “natural isotopic composition” refers to the naturally occurring isotopic composition or abundance for a given atom. Atoms containing their natural isotopic composition may also be referred to herein as “non-enriched” atoms. Unless otherwise designated, the atoms of the compounds recited herein are meant to represent any stable isotope of that atom. For example, unless otherwise stated, when a position is designated specifically as “H” or “hydrogen,” the position is understood to have hydrogen at its natural isotopic composition.

The term “isotopically enriched,” as used herein, and unless otherwise specified, refers to an atom having an isotopic composition other than the natural isotopic composition of that atom. “Isotopically enriched” may also refer to a compound containing at least one atom having an isotopic composition other than the natural isotopic composition of that atom.

The terms “medical food” or “clinical food” as used herein, generally refers to an ingestible composition that includes an active ingredient, such as a compound described herein or other composition described herein, in addition to one or more of digestible fats, carbohydrates and proteins. Medical foods or clinical foods may be prescribed and/or administered to a subject to address a specific patient population or condition, such as, for example, a specific deficiency or deficiency syndrome.

“NAC” means N-Acetylcysteine.

“Oxidative stress” means an imbalance between the production of free radicals and the ability of the biological system to counteract or detoxify their harmful effects through neutralization by antioxidants.

The term “pharmaceutically acceptable,” as used herein, generally refers to those properties and/or substances that are acceptable to the patient from a pharmacological/toxicological point of view and to the manufacturing pharmaceutical chemist from a physical/chemical point of view regarding composition, formulation, stability, patient acceptance, and bioavailability.

“Phosphate substituted form” means that any unsubstituted hydroxy group of the compound is substituted with a phosphate group, —P(O)(OH)₂ or —PO₃²⁻.

“Prophylactic use” or “protecting against,” utilizing the compounds discussed herein, is defined as using one or more of the compounds discussed herein as a prophylaxis against inflammation and/or oxidative stress (e.g. to prevent the deleterious effects of inflammation and/or oxidative stress or one or more symptoms of inflammation and/or oxidative stress) prior to exposure to factors which may result in inflammation and/or oxidative stress. Prophylactic use of the compounds and methods of the invention would include, in some embodiments, administering one or more of the compounds described herein to patients undergoing a medical treatment which may damage or injure the patient's liver, kidney, eyes, or skin, where the compound or compounds are administered prior to the medical treatment. Prevention includes complete prevention as well as partial prevention.

A “prophylactically effective amount” of a compound is an amount of the compound, which, when administered to a subject prior to exposure to a factor which may result in inflammation and/or oxidative stress, is sufficient to prevent one or more deleterious effects of inflammation and/or oxidative stress, or the clinical manifestation of inflammation and/or oxidative stress, or to prevent the manifestation of one or more adverse symptoms of inflammation and/or oxidative stress. A prophylactically effective amount can be given in one or more administrations.

“Prophylaxis” against injury or damage caused by inflammation and/or oxidative stress with the compounds and methods discussed herein is defined as administering one or more of the compounds discussed herein, with or without additional therapeutic agents, prior to exposure to factors which may result in inflammation and/or oxidative stress, in order to reduce or eliminate either the deleterious effects of inflammation and/or oxidative stress or one or more symptoms of inflammation and/or oxidative stress, or to retard the progression of the deleterious effects of inflammation and/or oxidative stress or of one or more symptoms of inflammation and/or oxidative stress, or to reduce the severity of the deleterious effects of inflammation and/or oxidative stress or of one or more symptoms of inflammation and/or oxidative stress, or to prevent the clinical manifestation of inflammation and/or oxidative stress, or to prevent the manifestation of adverse symptoms of inflammation and/or oxidative stress. Symptoms may include e.g. clinical symptoms and/or indicators or biomarkers of the disease state.

The term “quinone” designates a fully conjugated cyclic dione. “Hydroquinone” indicates the reduced form of the quinone (reduced by two electrons from the quinone form). For example, the hydroquinone form of Formula II is:

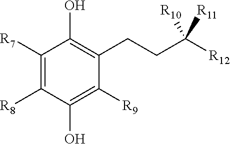

The term “synthetic,” as used herein, refers to compounds that have been made by chemical synthesis in a man-made setting, i.e., compounds that are prepared by reaction of other compounds, such as through the chemical conversion or chemical modification of one or more precursor compounds, in a man-made setting. Such synthetic compounds exclude compounds that are isolated or purified from a natural source or from an organism.

The term “non-plant based,” as used herein, refers to a compound that is not isolated from a plant source and is not produced from a tocopherol or tocotrienol that has been isolated from a plant source. For example, a “non-plant based alpha tocopherol” excludes alpha tocopherol that has been synthesized by methylating beta-, gamma-, and/or delta-tocopherols that were isolated from a plant source. It further excludes alpha-tocopherol that was produced by hydrogenation of alpha-tocotrienol that was isolated from a plant source. Because plants have a preference for C12 over C13, a compound that is produced by a plant will typically have a higher C12/C13 ratio than a compound that is not produced by a plant. In some embodiments, a non-plant based compound of the present disclosure has a C12/C13 molar ratio of less than about 99:1. In some embodiments, a non-plant based compound of the present disclosure has a C12/C13 molar ratio of less than about 95:1, 90:1, 80:1, 70:1, 60:1, or 50:1. In some embodiments, a non-plant based compound of the present disclosure has a C12/C13 molar ratio of greater than about 99:1. In some embodiments, a non-plant based compound of the present disclosure has a C12/C13 molar ratio of greater than about 95:about 1, about 90:about 1, about 80:about 1, about 70:about 1, about 60: about 1, or about 50:about 1.

The term “alpha-tocopherol activity” indicates the activity in international units. The different stereoisomers have different activity levels, as based on fertility enhancement by the prevention of miscarriages in pregnant rats. 1 IU is defined as 0.667 mg of RRR-alpha tocopherol.

The term “asymmetric carbon,” “chiral carbon,” or “stereocenter,” as used in reference to a carbon in a compound described herein, generally refers to a carbon atom that is attached to four different types of atoms or groups of atoms. A chiral carbon does not have a plane of symmetry, and thus, a compound that contains a chiral carbon does not have a plane of symmetry. An asymmetric carbon, chiral carbon, or stereocenter may be designated as having an R configuration or an S configuration, according to the (R)/(S) notation or the Cahn-Ingold Prelog Rules. In some embodiments, a compound may have an R stereocenter, an S stereocenter, or a combination of R and S stereocenters. In some embodiments, a compound that has a stereocenter may rotate plane polarized light.

The term “racemic mixture,” as used herein, generally refers to a substantially equal mixture of enantiomers of a chiral molecule or compound

The term “stereoisomer,” as used herein, generally refers to a single compound of all isomeric compounds that have the same sequence of bonded atoms and differ only in the three-dimensional orientation of their atoms in space. Stereoisomers may due to chiral carbons, or to cis-trans orientation of double bonds. For example, if a compound has three stereocenters, the compound has a possibility of eight diastereomers, or eight stereoisomers. As used herein, a stereoisomer generally refers to a single compound of the possible eight stereoisomers.

The term “enantiomer,” as used herein, generally refers to one of two stereoisomers that are mirror images of each other, and that are not superimposable on each other.

The term “enantiomeric ratio,” as used herein, generally refers to the ratio of a compound to its enantiomer. For example, if a solution contains 70% of the R isomer and 30% of the S isomer, the enantiomeric ratio, or e.r., will be 7:3 (R:S). If a solution contains a precise racemic mixture, i.e. 50% of the R isomer and 50% of the S isomer, the enantiomeric ratio will be 1:1 (R:S).

The term “diastereomeric ratio,” as used herein, generally refers to the ratio of a compound to one or more of its diastereomers. For example, alpha-tocopherol has three stereocenters. Thus, when referenced herein, an equal mixture of all eight potential alpha-tocopherol stereoisomers having chiral centers with configurations of -RRR, -RRS, -RSR, -RSS, -SSS, -SSR, -SRS, and -SRR, the diastereomeric ratio of the mixture is 1:1:1:1:1:1:1:1.

The compounds of the present disclosure may contain one or more asymmetric centers and/or one or more carbon-carbon double bonds, and may thus give rise to enantiomers, diastereomers, and other stereoisomeric forms that are defined, in terms of absolute stereochemistry, as (R) or (S), or as (E) or (Z). The present disclosure is meant to include all such possible isomers, as well as their racemic and optically pure forms.

The term “subject,” as used herein, generally includes humans of any age group, e.g., a pediatric subject (e.g., infant, child or adolescent) or adult subject (e.g., young adult, middle-aged adult or senior adult) and/or other primates (e.g., cynomolgus monkeys or rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. The methods described herein can be useful in both human therapeutics and veterinary applications. In some embodiments, the subject is a mammal, and in some embodiments, the subject is human. In some embodiments, a composition of the present disclosure is administered to a subject in need thereof. Also included is a “subject,” “individual,” or “patient,” which includes an individual organism, preferably a vertebrate, more preferably a mammal, most preferably a human.

“Therapeutic use” of the compounds discussed herein is defined as using one or more of the compounds discussed herein to treat inflammation and/or oxidative stress, wherein the subject has one or more symptoms or indications of inflammation and/or oxidative stress, or wherein the subject has been exposed to a factor which may result in inflammation and/or oxidative stress.

A “therapeutically effective amount” of a compound is an amount of the compound, which, when administered to a subject, is sufficient to reduce or eliminate either the deleterious effects of inflammation and/or oxidative stress or one or more symptoms of inflammation and/or oxidative stress, or to retard the progression of the deleterious effects of inflammation and/or oxidative stress or of one or more symptoms of inflammation and/or oxidative stress, or to reduce the severity of the deleterious effects of inflammation and/or oxidative stress or of one or more symptoms of inflammation and/or oxidative stress, or to suppress the deleterious effects of inflammation and/or oxidative stress or of one or more symptoms of inflammation and/or oxidative stress. A therapeutically effective amount can be given in one or more administrations

“Co-administration” includes simultaneous and sequential administration of a drug that may cause solid organ injury and a compound of Formula (I) or (II) as described herein in any embodiments. In some embodiments, the drug and compound of Formula (I) or Formula (II) are administered simultaneously in the same composition, simultaneously in separate compositions, or sequentially in separate compositions.

In some embodiments, when it is stated that an “additional material” or “member” has a weight % concentration of 1-99% of the one or more compounds, unless specifically specified otherwise, the weight % is calculated as [(weight of the additional material or member)/(weight of the one or more compounds)]×100. In some embodiments, when it is stated that an “additional material” or “member” has a weight % concentration of 1-99% of the one or more compounds, unless specifically specified otherwise, the weight % is calculated as [(weight of the additional material or member)/(weight of the additional material or member+weight of the one or more compounds)]×100.

In some embodiments, when the term “% by weight of the composition” is used, unless specifically specified otherwise, the % by weight of the composition is calculated as [(weight of the recited component)/(total weight of all components in the composition)]×100. In some embodiments, the weight of the composition excludes any carrier, excipient, diluent, adjuvant, and vehicle and the like.

In some embodiments, “age-related condition” means a condition where the likelihood of the condition occurring in a subject increases with age. In some embodiments, age-related condition means a condition associated with increasing age. In some embodiments, the age-related condition is selected from the group consisting of age-related eye disorders (AREDS), age-related macular degeneration, and age-related muscle loss (sarcopenia).

The term “treat” or “treating” or “treatment” as used herein, may include preventing a disease-state from occurring in a mammal, inhibiting a disease state, relieving or mitigating the disease state, and retarding the progression of the disease state. Treating also may include the amelioration of a symptom of a disease (e.g., lessen the pain or discomfort), wherein such amelioration may or may not be directly affecting the disease (e.g., cause, transmission, expression, etc.). Symptoms may also include one or more biomarkers or indicators of the disease state. Treating inflammation and/or oxidative stress with the compounds and methods discussed herein includes administering one or more of the compounds discussed herein, with or without additional therapeutic agents, in order to reduce or eliminate either the deleterious effects of inflammation and/or oxidative stress or one or more symptoms of inflammation and/or oxidative stress, or to retard the progression of the deleterious effects of inflammation and/or oxidative stress or of one or more symptoms of inflammation and/or oxidative stress, or to reduce the severity of the deleterious effects of inflammation and/or oxidative stress or of one or more symptoms of inflammation and/or oxidative stress, or to suppress the clinical manifestation of inflammation and/or oxidative stress, or to suppress the manifestation of adverse symptoms of inflammation and/or oxidative stress.

The term “substantially pure,” as used herein, refers to compositions that are enriched with a specific stereoisomer or stereoisomers of a particular compound, relative to other stereoisomers of the compound, i.e. purity of the specific stereoisomer(s) is measured relative to the amount of the other stereoisomers of the compound. In some embodiments, a composition that is substantially pure as to a specific stereoisomer or stereoisomers of a particular compound may comprise other stereoisomers of the compound in an amount not to exceed about 10% by weight of the total amount of all stereoisomers of the compound that are present in the composition (“purity is at least about 90%”). For instance, where a composition is recited as comprising substantially pure RRR-alpha-tocopherol, it will be understood that the total amount of all other alpha-tocopherol stereoisomers (RRS, RSR, SRR, SSR, SRS, RSS, SSS) in the composition will be less than about 10% by weight of the total amount of alpha-tocopherol present in said composition. For example, where a composition is recited as comprising substantially pure alpha-tocopherol having an R configuration at the 2-position of the chromanol ring, it will be understood that the total amount of all other alpha-tocopherol stereoisomers (SRR, SSR, SRS, SSS) in the composition will be less than about 10% by weight of the total amount of alpha-tocopherol present in said composition. In some embodiments, a composition that is substantially pure as to a specific stereoisomer or stereoisomers of a particular compound may comprise other stereoisomers of the compound in an amount not to exceed about 5% by weight of the total amount of all stereoisomers of the compound that are present in the composition (“purity is at least about 95%”). In some embodiments, a composition that is substantially pure as to a specific stereoisomer or stereoisomers of a particular compound may comprise other stereoisomers of the compound in an amount not to exceed about 4% by weight of the total amount of all stereoisomers of the compound that are present in the composition (“purity is at least about 96%”). In some embodiments, a composition that is substantially pure as to a specific stereoisomer or stereoisomers of a particular compound may comprise other stereoisomers of the compound in an amount not to exceed about 3% by weight of the total amount of all stereoisomers of the compound that are present in the composition (“purity is at least about 97%”). In some embodiments, a composition that is substantially pure as to a specific stereoisomer or stereoisomers of a particular compound may comprise other stereoisomers of the compound in an amount not to exceed about 2% by weight of the total amount of all stereoisomers of the compound that are present in the composition (“purity is at least about 98%”). In some embodiments, a composition that is substantially pure as to a specific stereoisomer or stereoisomers of a particular compound may comprise other stereoisomers of the compound in an amount not to exceed about 1% by weight of the total amount of all stereoisomers of the compound that are present in the composition (“purity is at least about 99%”). In some embodiments, a composition that is substantially pure as to a specific stereoisomer or stereoisomers of a particular compound may comprise other stereoisomers of the compound in an amount not to exceed about 0.5% by weight of the total amount of all stereoisomers of the compound that are present in the composition (“purity is at least about 99.5%”). When determining whether a stereoisomer is “substantially pure,” only the amounts of the synthetic, non-plant based stereoisomers are considered. For example, when determining the purity of (R,R,R)-alpha-tocopherol in a food, only the amounts of the various stereoisomers of synthetic, non-plant based alpha-tocopherol are considered; alpha-tocopherol that is naturally present in the food is not used in the determination of substantial purity.

In a first aspect, provided herein is a method of treating or protecting against inflammation in a biological system comprising: administering a therapeutically effective amount or prophylactically effective amount of a compound to a biological system in need thereof, wherein the compound is according to the formula:

(a)

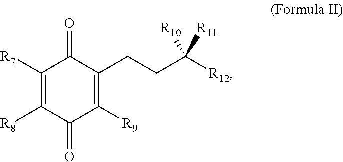

or the hydroquinone form thereof;
wherein:
R₇, R₈, and R₉ are independently hydrogen, C₁-C₆ alkyl, or C₁-C₆ alkoxy;
R₁₀ and R₁₁ are independently selected from the group consisting of hydrogen, methyl, and hydroxyl, with the proviso that only one of R₁₀ and
R₁₁ may be hydroxyl; and
R₁₂ is a C₁-C₁₃-alkyl or C₂-C₁₃-alkenyl group, wherein the C₁-C₁₃-alkyl and C₂-C₁₃-alkenyl groups are optionally substituted with one or more groups selected from the group consisting of: C₁-C₃ alkyl, halo, C₁-C₆ alkoxy, C₁-C₆ haloalkoxy, hydroxyl, and carboxylic acid; or

(b)

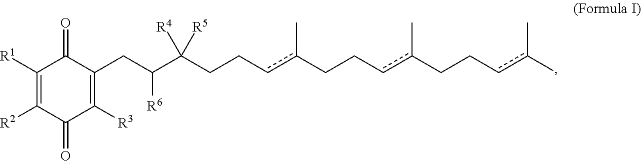

or the hydroquinone form thereof;
wherein:
each bond indicated with a dashed line is independently a single bond or a double bond;
R¹, R², and R³ are independently selected from H, (C₁-C₄)-alkyl, (C₁-C₄)-haloalkyl, —CN, —F, —Cl, —Br, and —I; and
R⁴ and R⁵ are independently selected from hydroxy and (C₁-C₄)-alkyl, and R⁶ is hydrogen; or
R⁴ is (C₁-C₄)-alkyl, and R⁵ and R⁶ are hydrogen; or
R⁴ is (C₁-C₄)-alkyl, and R⁵ and R⁶ together form the second bond of a double bond between the carbon atoms to which they are attached;
   or a stereoisomer, mixtures of stereoisomers, salt, phosphate substituted form, crystalline form, non-crystalline form, isotopologue, deuterated form, hydrate, or solvate thereof. In some embodiments of the method of treating or protecting against inflammation in a biological system, the compound is of Formula II, or the hydroquinone form thereof. In some embodiments of the method of treating or protecting against inflammation in a biological system, the compound is of Formula I, or the hydroquinone form thereof. In some instances, R₇, R₈, and R₉ in Formula II are independently hydrogen, C₁-C₃ alkyl, or C₁-C₃ alkoxy. In some instances, R₇, R₈, and R₉ in Formula II are independently hydrogen, methyl, or methoxy. In some instances, R₇, R₈, and R₉ in Formula II are independently hydrogen or methyl. In some instances, R₇, R₈, and R₉ in Formula II are methyl. In some instances of the method, in Formula II, one of R₁₀ and R₁₁ is hydroxyl and the other is methyl. In some instances of the method, in Formula II, R₁₀ and R₁₁ are both hydrogen. In some instances of the method, in Formula II, the C₁-C₁₃-alkyl or C₂-C₁₃-alkenyl groups are substituted with one or more groups selected from the group consisting of: C₁-C₃ alkyl, halo, C₁-C₆ alkoxy, C₁-C₆ haloalkoxy, hydroxyl, and carboxylic acid groups. In some instances of the method, in Formula II, the C₁-C₁₃-alkyl and C₂-C₁₃-alkenyl groups are substituted with one or more methyl groups. In some instances of the method, in Formula II, R₁₂ is C₁-C₁₃-alkyl substituted with one or more halo groups. In some instances of the method, in Formula II, R₁₂ is C₁-C₁₃-alkyl substituted with C₁-C₆ alkoxy. In some instances of the method, in Formula II, R₁₂ is C₁-C₁₃-alkyl substituted with C₁-C₄ alkoxy. In some instances of the method, in Formula II, R₁₂ is C₁-C₁₃-alkyl substituted with C₁-C₃ haloalkoxy. In some instances of the method, in Formula II, the C₁-C₁₃-alkyl and C₂-C₁₃-alkenyl groups are unsubstituted. In some instances of the method, in Formula II, R₁₂ is unsubstituted C₅-C₉-alkyl. In some instances of the method, in Formula II, the compound is in the quinone form. In some instances of the method, in Formula II, the compound is in the hydroquinone form. In some embodiments of the method of treating or protecting against inflammation in a biological system, the compound administered is selected from the group consisting of alpha-tocotrienol quinone, beta-tocotrienol quinone, gamma-tocotrienol quinone, delta-tocotrienol quinone, alpha-tocopherol quinone, beta-tocopherol quinone, gamma-tocopherol quinone, delta-tocopherol quinone, and the corresponding hydroquinone forms thereof, and stereoisomers, mixtures of stereoisomers, hydrates, and solvates thereof. In some embodiments of the method of treating or protecting against inflammation in a biological system, the compound administered is selected from the group consisting of alpha-tocotrienol quinone, and stereoisomers and mixtures of stereoisomers thereof. In some embodiments of the method of treating or protecting against inflammation in a biological system, the compound administered is selected from the group consisting of alpha-tocotrienol hydroquinone, and stereoisomers and mixtures of stereoisomers thereof. In some embodiments of the method of treating or protecting against inflammation in a biological system, the compound administered is selected from the group consisting of alpha-tocopherol quinone, and stereoisomers and mixtures of stereoisomers thereof. In some embodiments of the method of treating or protecting against inflammation in a biological system, the compound administered is selected from the group consisting of alpha-tocopherol hydroquinone, and stereoisomers and mixtures of stereoisomers thereof. In some embodiments of the method of treating or protecting against inflammation in a biological system, when the compound administered is a tocopherol quinone or hydroquinone form thereof, it has (R,R,R) stereochemistry, and when the compound is a tocotrienol quinone or hydroquinone form thereof, the compound has (R,E,E) stereochemistry. In some embodiments of the method of treating or protecting against inflammation in a biological system, the compound administered is

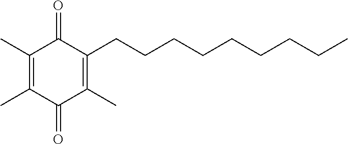 [SMILES: CCCCCCCCCC1=C(C)C(=O)C(C)=C(C)C1=O]

or the hydroquinone form thereof. In some embodiments of the method of treating or protecting against inflammation in a biological system, the compound administered is selected from the group consisting of:

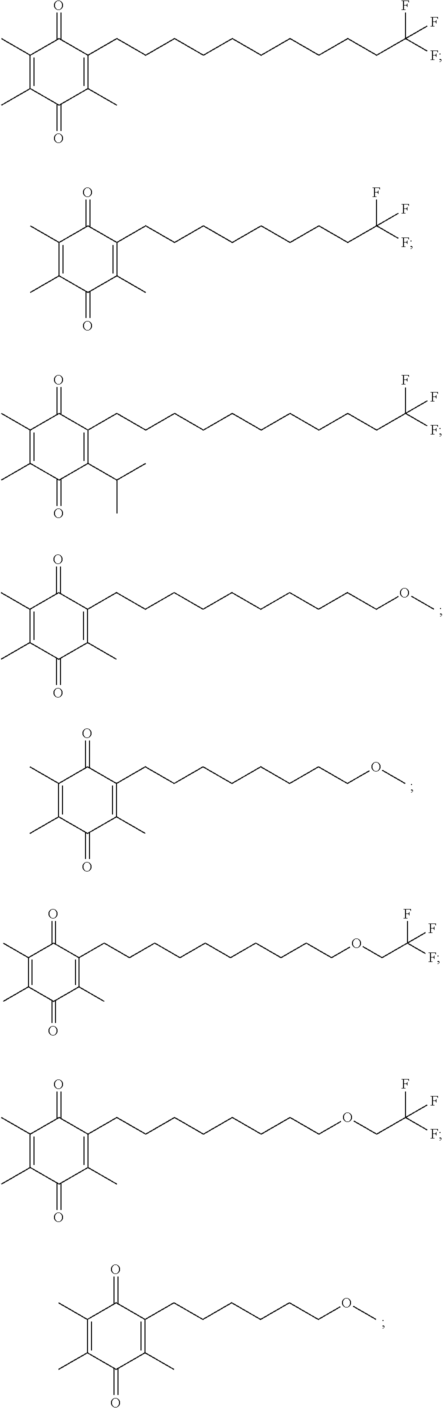

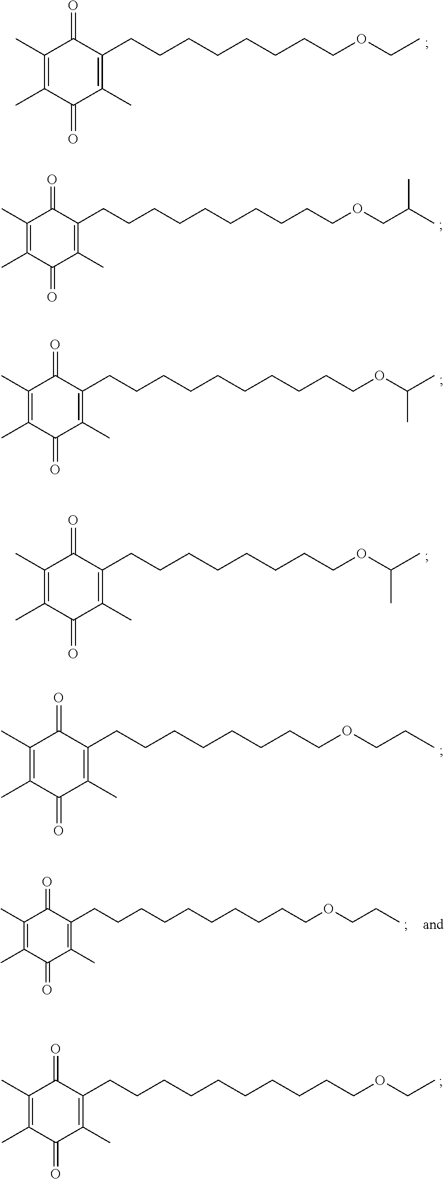

and the hydroquinones thereof. In some embodiments of the method of treating or protecting against inflammation in a biological system, administering the compound is achieved in a medical food or ingestible supplement. In some embodiments of the method of treating or protecting against inflammation in a biological system, administering the compound is achieved in a pharmaceutical composition comprising the compound and one or more pharmaceutically acceptable carriers, vehicles, and/or excipients. In some embodiments of the method of treating or protecting against inflammation in a biological system, administering the compound is done orally. In some embodiments of the method of treating or protecting against inflammation in a biological system, administering the compound is done via injection. In some embodiments of the method of treating or protecting against inflammation in a biological system, administering the compound is done topically. In some embodiments of the method of treating or protecting against inflammation in a biological system, administering is done in conjunction with NAC and/or DHA. In such embodiments, the NAC and/or DHA has a weight % concentration of 1-99% of the composition administered. In some embodiments of the method of treating or protecting against inflammation in a biological system, administering is done in conjunction with one or more compounds selected from the group consisting of: (R,R,R)-alpha-tocopherol, (R,R,R)-beta-tocopherol, (R,R,R)-gamma-tocopherol, (R,R,R)-delta-tocopherol, (R,E,E)-alpha-tocotrienol, (R,E,E)-beta-tocotrienol, (R,E,E)-gamma-tocotrienol, and (R,E,E)-delta-tocotrienol. In some embodiments of the method of treating or protecting against inflammation in a biological system, the biological system is a human subject. In some embodiments of the method of treating or protecting against inflammation in a biological system, the administering comprises about 100 mg to about 500 mg/kg of the compound per kg of body weight. In some embodiments of the method of treating or protecting against inflammation in a biological system, the administering comprises about 100 mg to about 300 mg/kg of the compound per kg of body weight. In some embodiments of the method of treating or protecting against inflammation in a biological system, the administering comprises about 100 mg to about 200 mg/kg of the compound per kg of body weight. In some embodiments of the method of treating or protecting against inflammation in a biological system, the administering is performed prior to inflammation. In some embodiments of the method of treating or protecting against inflammation in a biological system, the administering is performed after inflammation has occurred. In some embodiments the method is for treating the inflammation. In some embodiments the method is for protecting against the inflammation. In some embodiments of the method of treating or protecting against inflammation in a biological system, the inflammation is liver or kidney inflammation. In some embodiments of the method of treating or protecting against inflammation in a biological system, the inflammation is liver inflammation. In some of such embodiments, the liver inflammation is due to drug-induced liver injury. In certain cases, the drug induced liver injury is due to acetaminophen. In certain cases, the drug induced liver injury is due to cisplatin. In certain instances the inflammation is kidney inflammation. In certain cases the kidney inflammation is due to drug-induced kidney injury. In certain cases the inflammation is heart inflammation. In certain cases the inflammation is endothelial cell inflammation. In certain cases the endothelial cell is a heart cell. In any of the embodiment described above, the inflammation is not associated with one or more of: diabetes, liver damage, insulin resistance, nephropathy, acute or chronic renal injury due to exposure to nephrotoxic agents, drug induced peripheral neuropathy, serious atopic dermatitis, psoriasis, eczema, cyanide poisoning, decreased renal function, acute lung injury, calciphylaxis in blood vessels, kidney toxicity in cancer therapy, bacterial infection, fungal infection, ulcerative colitis, inflammatory bowel disease, hypertension, proteinuria, ischemia, hypoxia, atherosclerosis, uterus obstruction, hyperalgesia, lactic acidosis, Kearns-Sayre Syndrome (KSS), and primary progressive multiple sclerosis. In some instances, a mean liver enzyme concentration in serum is reduced. In some instances, a liver enzyme concentration in serum is reduced. In some instances, a mean liver enzyme concentration in serum is reduced by at least about 40%, 50%, 60%, 70%, 80%, 90%, or 100%. In some cases, a liver enzyme concentration in serum is reduced to a concentration less than about 3200 U/L, less than about 3000 U/L, less than about 2800 U/L, less than about 2600 U/L, less than about 2400 U/L, less than about 2200 U/L, less than about 2000 U/L, less than about 1800 U/L, less than about 1600 U/L, less than about 1400 U/L, less than about 1200 U/L, less than about 1000 U/L, less than about 800 U/L, less than about 600 U/L, less than about 400 U/L, less than about 200 U/L, or less than about 100 U/L. In some instances, the liver enzyme is Alanine Transaminase (ALT) or Aspartate Transaminase (AST). In some instances, the serum concentration of a pro-inflammatory cytokine is suppressed. In such cases, the pro-inflammatory cytokine is selected from the group consisting of IL-6, IL-8, and MCP-1. In some of such embodiments, the pro-inflammatory cytokine is selected from the group consisting of IL-6, IL-8, and TNF-α. In some embodiments, the serum concentration of a pro-inflammatory cytokine is reduced. In some embodiments, the serum concentration of the pro-inflammatory cytokine is reduced by at least about 40%, 50%, 60%, 70%, 80%, 90%, or 100%. In some cases, the pro-inflammatory cytokine is selected from the group consisting of IL-6, IL-8 and MCP-1. In some embodiments, the pro-inflammatory cytokine is selected from the group consisting of IL-6, IL-8 and TNF-α. In some embodiments, the suppressing is dose dependent.

In another aspect, provided herein is a method of reducing drug-induced solid organ injury, comprising co-administering to a subject in need thereof: (a) a drug that may cause solid organ injury, and (b) a compound comprising the formula:

(a)

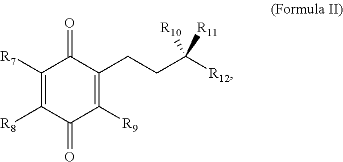

or the hydroquinone form thereof;
wherein:
R₇, R₈, and R₉ are independently hydrogen, C₁-C₆ alkyl, or C₁-C₆ alkoxy;
R₁₀ and R₁₁ are independently selected from the group consisting of hydrogen, methyl, and hydroxyl, with the proviso that only one of R₁₀ and R₁₁ may be hydroxyl; and
R₁₂ is a C₁-C₁₃-alkyl or C₂-C₁₃-alkenyl group, wherein the C₁-C₁₃-alkyl and C₂-C₁₃-alkenyl groups are optionally substituted with one or more groups selected from the group consisting of: C₁-C₃ alkyl, halo, C₁-C₆ alkoxy, C₁-C₆ haloalkoxy, hydroxyl, and carboxylic acid; or

(b)

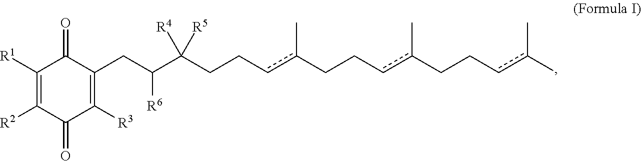

or the hydroquinone form thereof;
wherein:
each bond indicated with a dashed line is independently a single bond or a double bond;
R¹, R², and R³ are independently selected from H, (C₁-C₄)-alkyl, (C₁-C₄)-haloalkyl, —CN, —F, —Cl, —Br, and —I; and
R⁴ and R⁵ are independently selected from hydroxy and (C₁-C₄)-alkyl, and R⁶ is hydrogen; or
R⁴ is (C₁-C₄)-alkyl, and R⁵ and R⁶ are hydrogen; or
R⁴ is (C₁-C₄)-alkyl, and R⁵ and R⁶ together form the second bond of a double bond between the carbon atoms to which they are attached;
   or a stereoisomer, mixtures of stereoisomers, salt, phosphate substituted form, crystalline form, non-crystalline form, isotopologue, deuterated form, hydrate, or solvate thereof. In some instances of the method of reducing drug-induced solid organ injury, the co-administration is in a single pharmaceutical formulation. In some instances of the method of reducing drug-induced solid organ injury, the drug that may cause solid organ injury and the compound are administered sequentially or simultaneously in separate pharmaceutical formulations. In some instances of the method of reducing drug-induced solid organ injury, the drug that may cause solid organ injury is acetaminophen. In some instances of the method of reducing drug-induced solid organ injury, the drug that may cause solid organ injury is cisplatin. In some instances of the method of reducing drug-induced solid organ injury, the compound administered is of Formula II, or the hydroquinone form thereof. In some instances of the method of reducing drug-induced solid organ injury, the compound administered is of Formula I, or the hydroquinone form thereof. In some instances of the method of reducing drug-induced solid organ injury, in Formula II, R₇, R₈, and R₉ are independently hydrogen, C₁-C₃ alkyl, or C₁-C₃ alkoxy. In some instances of the method of reducing drug-induced solid organ injury, in Formula II, R₇, R₈, and R₉ are independently hydrogen, methyl, or methoxy. In some instances of the method of reducing drug-induced solid organ injury, in Formula II, R₇, R₈, and R₉ are independently hydrogen or methyl. In some instances of the method of reducing drug-induced solid organ injury, in Formula II, R₇, R₈, and R₉ are methyl. In some instances of the method of reducing drug-induced solid organ injury, in Formula II, one of R₁₀ and R₁₁ is hydroxyl and the other is methyl. In some instances of the method of reducing drug-induced solid organ injury, in Formula II, R₁₀ and R₁₁ are both hydrogen. In some instances of the method of reducing drug-induced solid organ injury, in Formula II, the C₁-C₁₃-alkyl and C₂-C₁₃-alkenyl groups in R¹² are substituted with one or more groups selected from the group consisting of: C₁-C₃ alkyl, halo, C₁-C₆ alkoxy, C₁-C₆ haloalkoxy, hydroxyl, and carboxylic acid groups. In some instances of the method of reducing drug-induced solid organ injury, in Formula II, the C₁-C₁₃-alkyl and C₂-C₁₃-alkenyl groups in R¹² are substituted with one or more methyl groups. In some instances of the method of reducing drug-induced solid organ injury, in Formula II, R₁₂ is C₁-C₁₃-alkyl substituted with one or more halo groups. In some instances of the method of reducing drug-induced solid organ injury, in Formula II, R₁₂ is C₁-C₁₃-alkyl substituted with one C₁-C₆ alkoxy. In some instances of the method of reducing drug-induced solid organ injury, in Formula II, R₁₂ is C₁-C₁₃-alkyl substituted with one C₁-C₄ alkoxy. In some instances of the method of reducing drug-induced solid organ injury, in Formula II, R₁₂ is C₁-C₁₃-alkyl substituted with one C₁-C₃ haloalkoxy. In some instances of the method of reducing drug-induced solid organ injury, in Formula II, the C₁-C₁₃-alkyl and C₂-C₁₃-alkenyl groups in R¹² are unsubstituted. In some instances of the method of reducing drug-induced solid organ injury, in Formula II, R₁₂ is unsubstituted C₅-C₉-alkyl. In some instances of the method of reducing drug-induced solid organ injury, the compound administered is in the quinone form. In some instances of the method of reducing drug-induced solid organ injury, the compound administered is in the hydroquinone form. In some instances of the method of reducing drug-induced solid organ injury, the compound administered is selected from the group consisting of alpha-tocotrienol quinone, beta-tocotrienol quinone, gamma-tocotrienol quinone, delta-tocotrienol quinone, alpha-tocopherol quinone, beta-tocopherol quinone, gamma-tocopherol quinone, delta-tocopherol quinone, and the corresponding hydroquinone forms thereof, and stereoisomers, mixtures of stereoisomers, hydrates, and solvates thereof. In some instances of the method of reducing drug-induced solid organ injury, in the compound administered is selected from the group consisting of alpha-tocotrienol quinone, and stereoisomers and mixtures of stereoisomers thereof. In some instances of the method of reducing drug-induced solid organ injury, the compound administered is selected from the group consisting of alpha-tocotrienol hydroquinone, and stereoisomers and mixtures of stereoisomers thereof. In some instances of the method of reducing drug-induced solid organ injury, the compound administered is selected from the group consisting of alpha-tocopherol quinone, and stereoisomers and mixtures of stereoisomers thereof. In some instances of the method of reducing drug-induced solid organ injury, the compound administered is selected from the group consisting of alpha-tocopherol hydroquinone, and stereoisomers and mixtures of stereoisomers thereof. In some instances of the method of reducing drug-induced solid organ injury, when the compound administered is a tocopherol quinone or hydroquinone form thereof, it has (R,R,R) stereochemistry, and when the compound is a tocotrienol quinone or hydroquinone form thereof, the compound has (R,E,E) stereochemistry. In some instances of the method of reducing drug-induced solid organ injury, in Formula II, the compound administered is

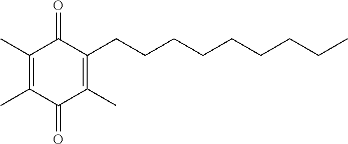 [SMILES: CCCCCCCCCC1=C(C)C(=O)C(C)=C(C)C1=O]

or the hydroquinone form thereof. In some instances of the method of reducing drug-induced solid organ injury, the compound administered is selected from the group consisting of:

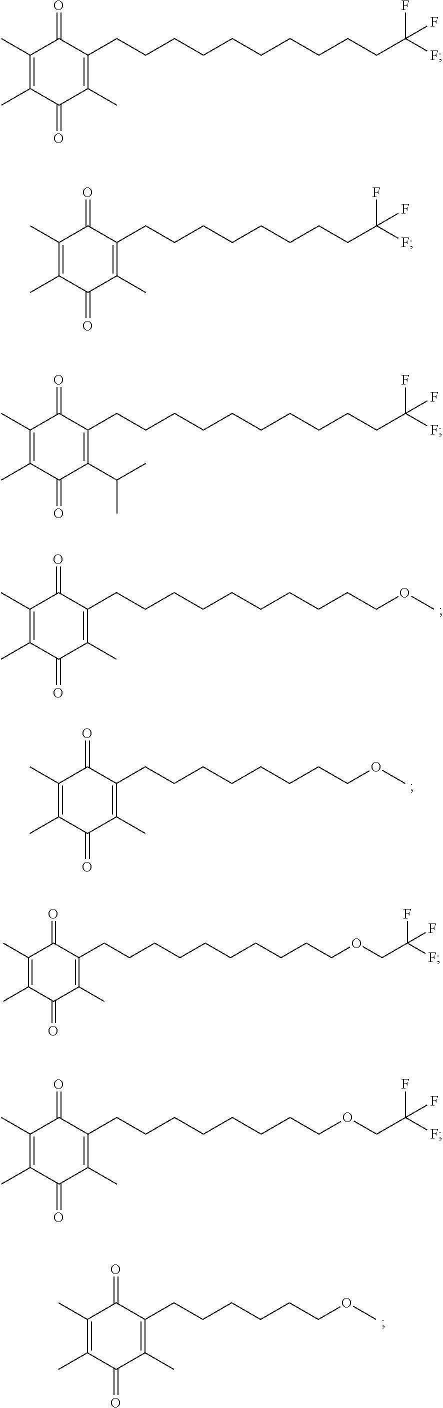

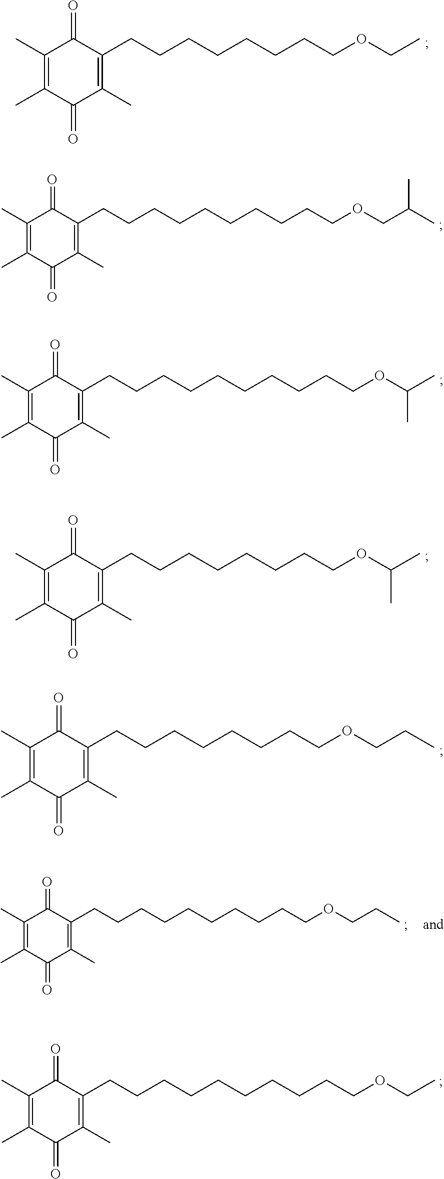

and the hydroquinones thereof. In some instances of the method of reducing drug-induced solid organ injury, administering the compound is achieved in a medical food or ingestible supplement. In some instances of the method of reducing drug-induced solid organ injury, administering the compound is achieved in a pharmaceutical composition comprising the compound and one or more pharmaceutically acceptable carriers, vehicles, and/or excipients. In some instances of the method of reducing drug-induced solid organ injury, administering the compound is done orally. In some instances of the method of reducing drug-induced solid organ injury, administering the compound is done via injection. In some instances of the method of reducing drug-induced solid organ injury, administering the compound is done topically. In some instances of the method of reducing drug-induced solid organ injury, administering is done in conjunction with NAC and/or DHA. In some instances of the method of reducing drug-induced solid organ injury, the NAC and/or DHA has a weight % concentration of 1-99% of the compound administered. In some instances of the method of reducing drug-induced solid organ injury, administering is done in conjunction with one or more compounds selected from the group consisting of: (R,R,R)-alpha-tocopherol, (R,R,R)-beta-tocopherol, (R,R,R)-gamma-tocopherol, (R,R,R)-delta-tocopherol, (R,E,E)-alpha-tocotrienol, (R,E,E)-beta-tocotrienol, (R,E,E)-gamma-tocotrienol, and (R,E,E)-delta-tocotrienol. In some instances of the method of reducing drug-induced solid organ injury, the subject is a human. In some instances of the method of reducing drug-induced solid organ injury, the administering comprises about 100 mg to about 500 mg/kg of the compound per kg of body weight. In some instances of the method of reducing drug-induced solid organ injury, the administering comprises about 100 mg to about 300 mg/kg of the compound per kg of body weight. In some instances of the method of reducing drug-induced solid organ injury, the administering comprises about 100 mg to about 200 mg/kg of the compound per kg of body weight. In some instances of the method of reducing drug-induced solid organ injury, the solid organ is liver or kidney. In some instances of the method of reducing drug-induced solid organ injury, the solid organ is liver. In some instances of the method of reducing drug-induced solid organ injury, the solid organ is kidney. In some instances of the method of reducing drug-induced solid organ injury, a mean liver enzyme concentration in serum is reduced. In some instances of the method of reducing drug-induced solid organ injury, a liver enzyme concentration in serum is reduced. In some instances of the method of reducing drug-induced solid organ injury, a mean liver enzyme concentration in serum is reduced by at least about 40%, 50%, 60%, 70%, 80%, 90%, or 100%. In some instances of the method of reducing drug-induced solid organ injury, a liver enzyme concentration in serum is reduced to a concentration less than about 3200 U/L, less than about 3000 U/L, less than about 2800 U/L, less than about 2600 U/L, less than about 2400 U/L, less than about 2200 U/L, less than about 2000 U/L, less than about 1800 U/L, less than about 1600 U/L, less than about 1400 U/L, less than about 1200 U/L, less than about 1000 U/L, less than about 800 U/L, less than about 600 U/L, less than about 400 U/L, less than about 200 U/L, or less than about 100 U/L. In some cases the liver enzyme is Alanine Transaminase (ALT) or Aspartate Transaminase (AST). In some instances of the method of reducing drug-induced solid organ injury, serum concentration of a pro-inflammatory cytokine is suppressed. In some instances of the method of reducing drug-induced solid organ injury, the pro-inflammatory cytokine is selected from the group consisting of IL-6, IL-8, and MCP-1. In some instances of the method of reducing drug-induced solid organ injury, the pro-inflammatory cytokine is selected from the group consisting of IL-6, IL-8, and TNF-α. In some instances of the method of reducing drug-induced solid organ injury, serum concentration of a pro-inflammatory cytokine is reduced. In some instances of the method of reducing drug-induced solid organ injury, serum concentration of the pro-inflammatory cytokine is reduced by at least about 40%, 50%, 60%, 70%, 80%, 90%, or 100%. In some cases the pro-inflammatory cytokine is selected from the group consisting of IL-6, IL-8 and MCP-1. In some cases, the pro-inflammatory cytokine is selected from the group consisting of IL-6, IL-8 and TNF-α.

In some or any embodiments, any of the methods of treating or protecting against inflammation or any of the methods of reducing drug-induced solid organ injury can be combined with any of the methods of identifying a biological system (in some embodiments, a human) at risk of or suffering from inflammation and/or combined with any of the methods of monitoring inflammation in a biological system (in some embodiments, a human). These combined methods can comprise: measuring the levels of one or more biomarker(s) selected from the group consisting of: ALT, AST, IL-6, IL-8, MCP-1, and TNF-α in a subject; and/or can comprise determining an inflammatory state of the biological system (in some embodiments, a human) from the measurement of the levels of one or more biomarker(s) relative to the normal levels; and/or can comprise identifying an abnormal level of one or more of the one or more biomarkers; and/or can comprise determining whether the one or more of the levels of the one or more biomarkers are above normal levels.

In a further aspect, provided herein is a method of treating inflammation, comprising: (a) measuring the levels of one or more biomarker(s) selected from the group consisting of: ALT, AST, IL-6, IL-8, MCP-1, and TNF-α in a subject, and (b) administering to the subject a compound or composition as described above and herein when the biomarker(s) indicates inflammation. In some instances of the method of treating inflammation, the inflammation is in a solid organ. In some instances of the method of treating inflammation, the solid organ is liver. In some instances of the method of treating inflammation, the one or more biomarker(s) are selected from the group consisting of ALT and AST. In some instances of the method of treating inflammation, the solid organ is kidney. In some instances, the inflammation is heart inflammation. In some instances of the method of treating inflammation, the inflammation is endothelial cell inflammation. In certain cases the endothelial cell is a heart cell. In some instances of the method of treating inflammation, the one or more biomarker(s) are selected from the group consisting of IL-6, IL-8, MCP-1, or TNF-α.

In some or any embodiments, provided is a method of monitoring inflammation in a biological system (in some embodiments, a human), comprising: (a) measuring the levels of one or more biomarker(s) selected from the group consisting of: ALT, AST, IL-6, IL-8, MCP-1, and TNF-α in a subject, and (b) determining an inflammatory state of the biological system (in some embodiments, a human) from the measurement of the levels of one or more biomarker(s) relative to the normal levels. In some or any embodiments, the inflammation is in a solid organ. In some or any embodiments, the solid organ is liver. In some or any embodiments, the one or more biomarker(s) are ALT or AST. In some or any embodiments, the solid organ is kidney. In some or any embodiments, the inflammation is endothelial cell inflammation. In some or any embodiments, the one or more biomarker(s) are IL-6, IL-8, MCP-1, or TNF-α. In some or any embodiments, the biological system (in some embodiments, a human) is monitored for inflammation before treatment for inflammation, where the treatment in some embodiments is a Compound of Formula (I) or (II). In some or any embodiments, the one or more biomarker(s) are IL-6, IL-8, MCP-1, or TNF-α. In some or any embodiments, the biological system (in some embodiments, a human) is monitored for inflammation after treatment for inflammation, where the treatment in some embodiments is a Compound of Formula (I) or (II).

In some or any embodiments, provided is a method of identifying a biological system (in some embodiments, a human) at risk of or suffering from inflammation, comprising: (a) measuring the levels of one or more biomarker(s) selected from the group consisting of: ALT, AST, IL-6, IL-8, MCP-1, and TNF-α in a subject, and (b) determining an inflammatory state of the biological system (in some embodiments, a human) from the measurement of the levels of one or more biomarker(s) relative to the normal levels. In some or any embodiments, the inflammation is in a solid organ. In some or any embodiments, the solid organ is liver. In some or any embodiments, the one or more biomarker(s) are ALT or AST. In some or any embodiments, the solid organ is kidney. In some or any embodiments, the inflammation is endothelial cell inflammation. In some or any embodiments, the one or more biomarker(s) are IL-6, IL-8, MCP-1, or TNF-α.

In some or any embodiments, provided is a method of treating inflammation in a biological system (in some embodiments, a human), comprising: (a) measuring the levels of one or more biomarker(s) selected from the group consisting of: ALT, AST, IL-6, IL-8, MCP-1, and TNF-α in a subject, (b) identifying an abnormal level of one or more of the one or more biomarkers, and (c) administering to the biological system (in some embodiments, a human) a compound of Formula (I) or (II) or a composition comprising a compound of Formula (I) or (II) when the biomarker(s) indicates inflammation. In some or any embodiments, the inflammation is in a solid organ. In some or any embodiments, the solid organ is liver. In some or any embodiments, the one or more biomarker(s) are ALT or AST. In some or any embodiments, the solid organ is kidney. In some or any embodiments, the inflammation is endothelial cell inflammation. In some or any embodiments, the one or more biomarker(s) are IL-6, IL-8, MCP-1, or TNF-α.

In some or any embodiments, provided is a method of treating inflammation in a biological system (in some embodiments, a human), comprising: (a) measuring the levels of one or more biomarker(s) selected from the group consisting of: ALT, AST, IL-6, IL-8, MCP-1, and TNF-α in a subject, (b) determining whether the one or more of the levels of the one or more biomarkers are above normal levels, and (c) administering to the biological system (in some embodiments, a human) a compound of Formula (I) or (II) or a composition comprising a compound of Formula (I) or (II) when the biomarker(s) indicates inflammation. In some or any embodiments, the inflammation is in a solid organ. In some or any embodiments, the solid organ is liver. In some or any embodiments, the one or more biomarker(s) are ALT or AST. In some or any embodiments, the solid organ is kidney. In some or any embodiments, the inflammation is endothelial cell inflammation. In some or any embodiments, the one or more biomarker(s) are IL-6, IL-8, MCP-1, or TNF-α.

As described throughout the instant disclosure, provided are methods where one or more biomarkers are measured and are then compared to models for values of such biomarkers to determine the inflammatory state of the biological system (e.g. human), e.g., is it deviating from a model, e.g., a normal or healthy value, or does it match a model associated with a particular inflammatory state, e.g., inflammation. The model may be a simple comparison of distinct biomarker values or may be a more complex mathematical model. In some embodiments, including any of the foregoing embodiments, the one or more biomarker level is compared to a control value which is drawn from healthy or normal biological systems. In some embodiments, including any of the foregoing embodiments, the control value is an age-matched control value. In some embodiments, including any of the foregoing embodiments, the control value is a BMI-matched control value. In some embodiments, including any of the foregoing embodiments, the control value is a sex-matched control value.

In some embodiments of the disclosure, provided is a composition comprising one or more compounds of Formula II.

Compounds for Use in Treatment or Prophylaxis of Inflammation and/or Oxidative Stress

The methods provided herein comprise the use of one or more compounds of Formula I as described herein, or Formula II as described herein, or a combination thereof.

Compounds of Formula II have the following structure:

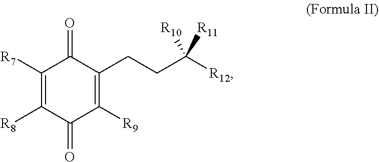

or the hydroquinone form thereof;
wherein: R₇, R₈, and R₉ are independently hydrogen, C₁-C₆ alkyl, or C₁-C₆ alkoxy; R₁₀ and R₁₁ are independently selected from the group consisting of hydrogen, methyl, and hydroxyl, with the proviso that only one of R₁₀ and R₁₁ may be hydroxyl; and Ria is a C₁-C₁₃-alkyl or C₂-C₁₃-alkenyl group, wherein the C₁-C₁₃-alkyl or C₂-C₁₃-alkenyl groups are optionally substituted with one or more groups selected from the group consisting of: C₁-C₃ alkyl, halo, C₁-C₆ alkoxy, C₁-C₆ haloalkoxy, hydroxyl, and carboxylic acid; or a stereoisomer, mixtures of stereoisomers, salt, phosphate substituted form, crystalline form, non-crystalline form, isotopologue, deuterated form, hydrate, or solvate thereof. Additional embodiments of Formula II are described herein.

Compounds for use in the invention further include one or more compounds:

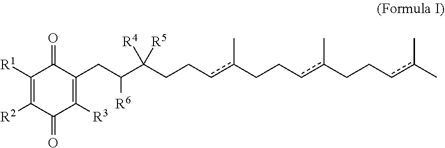

wherein:
each bond indicated with a dashed line is independently a single bond or a double bond;
R¹, R², and R³ are independently selected from H, (C₁-C₄)-haloalkyl, —CN, —F, —Cl, —Br, and —I; and
R⁴ and R⁵ are independently selected from hydroxy and (C₁-C₄)-alkyl, and R⁶ is hydrogen; or
R⁴ is (C₁-C₄)-alkyl, and R⁵ and R⁶ are hydrogen; or
R⁴ is (C₁-C₄)-alkyl, and R⁵ and R⁶ together form the second bond of a double bond between the carbon atoms to which they are attached;
   or a stereoisomer, mixtures of stereoisomers, prodrug, metabolite, salt, phosphate substituted form, crystalline form, non-crystalline form, isotopologue, deuterated form, hydrate, or solvate thereof or the hydroquinone form thereof.

Compounds for use in the invention also include one or more compounds of:

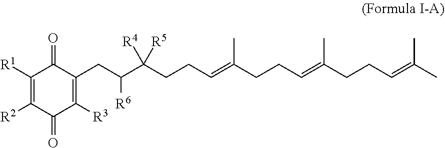

where the substituents are as indicated for Formula I; or a stereoisomer, mixtures of stereoisomers, prodrug, metabolite, salt, phosphate substituted form, crystalline form, non-crystalline form, isotopologue, deuterated form, hydrate, or solvate thereof;
or the hydroquinone form thereof.

Compounds for use in the invention also include one or more compounds:

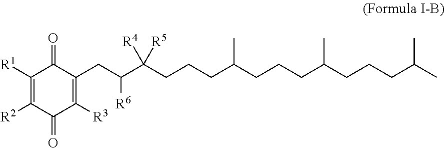

where the substituents are as indicated for; or a stereoisomer, mixtures of stereoisomers, prodrug, metabolite, salt, phosphate substituted form, crystalline form, non-crystalline form, isotopologue, deuterated form, hydrate, or solvate thereof;
or the hydroquinone form thereof.

Table 1 below indicates some embodiments of the present disclosure where the substitution pattern for alpha, beta, gamma, and delta tocotrienol quinone is set forth.

TABLE 1
Tocotrienol quinones

| 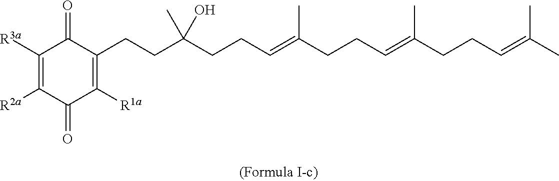 | | R^{1a} | R^{2a} | R^{3a} |
|---|---|---|---|---|
| Alpha- tocotrienol quinone | 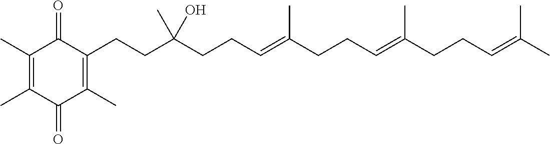 [SMILES: CC(C)=CCC/C(C)=C/CC/C(C)=C/CCC(C)(O)CCC1=C(C)C(=O)C(C)=C(C)C1=O] | methyl | methyl | methyl |
| Beta- tocotrienol quinone | 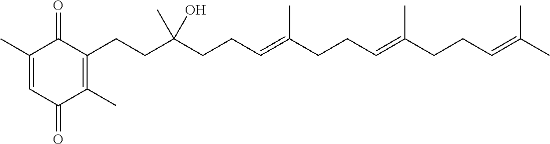 [SMILES: CC(C)=CCC/C(C)=C/CC/C(C)=C/CCC(C)(O)CCC1=C(C)C(=O)C=C(C)C1=O] | methyl | H | methyl |
| Gamma- tocotrienol quinone | 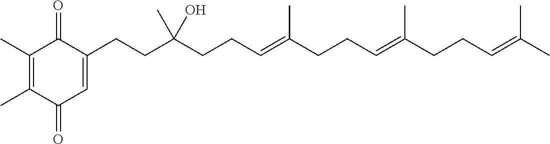 [SMILES: CC(C)=CCC/C(C)=C/CC/C(C)=C/CCC(C)(O)CCC1=CC(=O)C(C)=C(C)C1=O] | H | methyl | methyl |
| Delta- tocotrienol quinone | 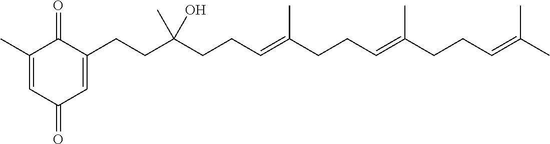 | H | H | methyl |

The tocotrienol quinones have a stereocenter at the 3-position on the hydrophobic tail. In some embodiments, the tocotrienol quinone has an R-configuration at the 3 position. In some embodiments, the tocotrienol quinone has an S-configuration at the 3-position. In some embodiments, the compound is REE-alpha-tocotrienol quinone where E indicates the configuration of the double bonds.

The same substitution pattern as that indicated above for tocotrienol quinones for methyl and hydrogen groups yield the alpha, beta, gamma, and delta tocopherol quinone variants. Thus, alpha tocopherol quinone is shown below:

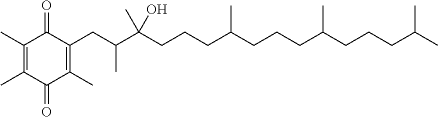 [SMILES: CC1=C(C)C(=O)C(CC(C)C(C)(O)CCCC(C)CCCC(C)CCCC(C)C)=C(C)C1=O]

The tocopherol quinones have a stereocenter at the 3-position, the 7-position, and the 11-position on the hydrophobic tail. Thus, 8 diastereomers are possible: (RRR), (RRS), (RSR), (SRR), (SSR), (SRS), (RSS), and (SSS). In some embodiments, the tocopherol quinone has an R-configuration at the 3 position. In some embodiments, the alpha tocopherol quinone has an R-configuration at the 3 position. In some embodiments, the compound is RRR-alpha-tocopherol quinone.

For the purposes of the present disclosure, REE-alpha tocotrienol quinone is also referred to as EP-743, or EPI-743, or 743. For the purposes of the present disclosure, RRR-alpha tocopherol quinone is also referred to as ATQ, EP-A0001, EPI-A0001, or A0001. A0001 is a viscous orange oil with no discernable taste. A0001 is infinitely miscible with other oils and practically insoluble in aqueous solvents.

U.S. Provisional Application No. 62/485,739, titled “VITAMIN E COMPOSITIONS AND METHODS OF USE THEREFOR”, filed on Apr. 14, 2017, U.S. Provisional Application No. 62/488,643, titled “VITAMIN E COMPOSITIONS AND METHODS OF USE THEREFOR”, filed on Apr. 21, 2017, and U.S. Nonprovisional Application No. ______, titled “VITAMIN E COMPOSITIONS AND METHODS OF USE THEREFOR”, filed on Apr. 14, 2018, all by the same Applicant, and which are incorporated by reference herein in their entireties, describe various synthetic, non-plant based tocopherol quinone, tocopherol hydroquinone, tocotrienol quinone, and tocotrienol hydroquinone compounds and compositions. Such compounds and compositions may be used in any of the methods described herein.

### Formulations

The compound compositions described above may, in some embodiments, be incorporated in a broader formulation or composition, e.g., formulated with other components, to provide for different applications and to serve different purposes, such as to improve bioavailability, improve storage, controlled release, solubility, mode of administration, and the like. Non-limiting examples of such compositions include medical foods and pharmaceutical formulations. The compositions may be formulated for internal or topical use.

In some cases, the compositions described herein may be co-administered, including as co-formulations, with other compositions comprising a therapeutic agent. In some embodiments, such other compositions are used in the treatment of certain disorders or conditions, in applications where such compositions have demonstrated efficacy, in order to improve that efficacy and/or reduce adverse side effects or reactions. For example, in some cases, the compositions described herein may be co-administered with one or more omega-3 fatty acids (also referred to as n-3 polyunsaturated fatty acids or n-3 PUFA), such as docosohexanoic acid (DHA) and/or eicosapentanoic acid (EPA), as well as CoQ10 or n-acetyl cysteine (NAC) in conditions that are typically treated using such compositions. Such co-administration may be employed, for example, in the treatment of liver diseases, e.g., non-alcoholic fatty liver disease (NAFLD), pediatric NAFLD, age-related conditions, e.g., age-related eye disorders (AREDS), age-related macular degeneration, age-related muscle loss (sarcopenia), skin disorders, e.g., rosacea, eczema, psoriasis, radiation damage, including acute skin damage caused by radiation therapy as well as transient radiation induced inflammation. Likewise, such co-administration may be used in cases where such n3-PUFAs are administered to enhance effectiveness of a given treatment, such as in chemotherapy. In addition, co-administration with antivirals, for example antivirals for hepatitis, and co-administration in conjunction with standard treatments for Wilson's disease and hemochromatosis to improve effectiveness.

In some embodiments, the compositions comprising one or more quinone or hydroquinone compounds as described herein (“Component A”) may be co-administered, including as co-formulations, with one or more of: alpha tocopherol, beta tocopherol, gamma tocopherol, delta tocopherol, alpha tocotrienol, beta tocotrienol, gamma tocotrienol, and delta tocotrienol (“Component B”). In some embodiments, such combination may be further co-administered, including as co-formulations, with Docosahexaenoic acid (DHA) (“Component C”). In some embodiments, a composition comprising alpha tocopherol quinone (“Component A1”) may be co-administered with, including as co-formulations, with alpha tocopherol (“Component B1”) and DHA wherein the alpha tocopherol and DHA may be formulated individually or together. In some embodiments, a composition comprising (R,R,R)-alpha tocopherol quinone (“Component A2”) may be co-administered with, including as co-formulations, with (R,R,R)-alpha tocopherol (“Component B2”) and DHA wherein the (R,R,R)-alpha tocopherol and DHA may be formulated individually or together.

In some embodiments, a composition comprising alpha tocotrienol quinone (“Component A3”) may be co-administered with, including as co-formulations, with alpha tocotrienol (“Component B3”) and DHA wherein the alpha tocotrienol and DHA may be formulated individually or together. In some embodiments, a composition comprising (R,E,E)-alpha tocotrienol quinone (“Component A4”) may be co-administered with, including as co-formulations, with (R,E,E)-alpha tocotrienol (“Component B4”) and DHA wherein the alpha tocotrienol and DHA may be formulated individually or together. In some embodiments, a formulation comprises alpha tocopherol quinone, alpha tocopherol, and DHA. In some embodiments, a formulation comprises (R,R,R)-alpha tocopherol quinone, (R,R,R)-alpha tocopherol, and DHA. In some embodiments, a formulation comprises alpha tocotrienol quinone, alpha tocotrienol, and DHA. In some embodiments, a formulation comprises (R,E,E)-alpha tocotrienol quinone, (R,E,E)-alpha tocotrienol, and DHA. In some or any embodiments, including any of the foregoing embodiments, the one or more quinone or hydroquinone compounds of Component A is according to Formula I, I-A, I-B, and/or I-C, or any a stereoisomer, mixtures of stereoisomers, hydroquinone form, prodrug, metabolite, salt, phosphate substituted form, crystalline form, non-crystalline form, isotopologue, deuterated form, hydrate, or solvate thereof, or combination thereof. In some or any embodiments, including any of the foregoing embodiments, the one or more quinone or hydroquinone compounds of Component A is according to Formula II, or any a stereoisomer, mixtures of stereoisomers, hydroquinone form, salt, phosphate substituted form, crystalline form, non-crystalline form, isotopologue, deuterated form, hydrate, or solvate thereof, or combination thereof. In some of any embodiments, including any of the foregoing embodiments, a composition comprises Component A and Component B, wherein Component A is about 0.01% to about 99.9%, about 0.1% to about 99.9%, about 1% to about 99.9%, about 5% to about 99.9%, about 10% to about 99.9%, about 15% to about 99.9%, about 20% to about 99.9%, about 25% to about 99.9%, about 30% to about 99.9%, about 35% to about 99.9%, about 40% to about 99.9%, about 45% to about 99.9%, about 50% to about 99.9%, about 55% to about 99.9%, about 60% to about 99.9%, about 65% to about 99.9%, about 70% to about 99.9%, about 75% to about 99.9%, about 80% to about 99.9%, about 85% to about 99.9%, about 90% to about 99.9%, about 95% to about 99.9%, about 1% to about 95%, about 1% to about 90%, about 1% to about 85%, about 1% to about 80%, about 1% to about 75%, about 1% to about 70%, about 1% to about 65%, about 1% to about 60%, about 1% to about 55%, about 1% to about 50%, about 1% to about 45%, about 1% to about 40%, about 1% to about 35%, about 1% to about 30%, about 1% to about 25%, about 1% to about 20%, about 1% to about 15%, about 1% to about 10%, about 1% to about 5%, about 10% to about 20%, about 20% to about 30%, about 30% to about 40%, about 40% to about 50%, about 50% to about 60%, about 60% to about 70%, about 70% to about 80%, about 80% to about 90%, about 90% to about 99.9%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% by weight of the total weight of Component A and Component B. In some of any embodiments, including any of the foregoing embodiments, a composition comprises Component A, Component B, and Component C, wherein Component A is about 0.01% to about 99.9%, about 0.1% to about 99.9%, about 1% to about 99.9%, about 5% to about 99.9%, about 10% to about 99.9%, about 15% to about 99.9%, about 20% to about 99.9%, about 25% to about 99.9%, about 30% to about 99.9%, about 35% to about 99.9%, about 40% to about 99.9%, about 45% to about 99.9%, about 50% to about 99.9%, about 55% to about 99.9%, about 60% to about 99.9%, about 65% to about 99.9%, about 70% to about 99.9%, about 75% to about 99.9%, about 80% to about 99.9%, about 85% to about 99.9%, about 90% to about 99.9%, about 95% to about 99.9%, about 1% to about 95%, about 1% to about 90%, about 1% to about 85%, about 1% to about 80%, about 1% to about 75%, about 1% to about 70%, about 1% to about 65%, about 1% to about 60%, about 1% to about 55%, about 1% to about 50%, about 1% to about 45%, about 1% to about 40%, about 1% to about 35%, about 1% to about 30%, about 1% to about 25%, about 1% to about 20%, about 1% to about 15%, about 1% to about 10%, about 1% to about 5%, about 10% to about 20%, about 20% to about 30%, about 30% to about 40%, about 40% to about 50%, about 50% to about 60%, about 60% to about 70%, about 70% to about 80%, about 80% to about 90%, about 90% to about 99.9%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% by weight of the total weight of Component A, Component B, and Component C. In some of any embodiments, including any of the foregoing embodiments, a composition comprises Component A, Component B, and Component C, wherein Component B is about 0.01% to about 99.9%, about 0.1% to about 99.9%, about 1% to about 99.9%, about 5% to about 99.9%, about 10% to about 99.9%, about 15% to about 99.9%, about 20% to about 99.9%, about 25% to about 99.9%, about 30% to about 99.9%, about 35% to about 99.9%, about 40% to about 99.9%, about 45% to about 99.9%, about 50% to about 99.9%, about 55% to about 99.9%, about 60% to about 99.9%, about 65% to about 99.9%, about 70% to about 99.9%, about 75% to about 99.9%, about 80% to about 99.9%, about 85% to about 99.9%, about 90% to about 99.9%, about 95% to about 99.9%, about 1% to about 95%, about 1% to about 90%, about 1% to about 85%, about 1% to about 80%, about 1% to about 75%, about 1% to about 70%, about 1% to about 65%, about 1% to about 60%, about 1% to about 55%, about 1% to about 50%, about 1% to about 45%, about 1% to about 40%, about 1% to about 35%, about 1% to about 30%, about 1% to about 25%, about 1% to about 20%, about 1% to about 15%, about 1% to about 10%, about 1% to about 5%, about 10% to about 20%, about 20% to about 30%, about 30% to about 40%, about 40% to about 50%, about 50% to about 60%, about 60% to about 70%, about 70% to about 80%, about 80% to about 90%, about 90% to about 99.9%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% by weight of the total weight of Component A, Component B, and Component C. In some of any embodiments, including any of the foregoing embodiments, a composition comprises Component A, Component B, and Component C, wherein Component C is about 0.01% to about 99.9%, about 0.1% to about 99.9%, about 1% to about 99.9%, about 5% to about 99.9%, about 10% to about 99.9%, about 15% to about 99.9%, about 20% to about 99.9%, about 25% to about 99.9%, about 30% to about 99.9%, about 35% to about 99.9%, about 40% to about 99.9%, about 45% to about 99.9%, about 50% to about 99.9%, about 55% to about 99.9%, about 60% to about 99.9%, about 65% to about 99.9%, about 70% to about 99.9%, about 75% to about 99.9%, about 80% to about 99.9%, about 85% to about 99.9%, about 90% to about 99.9%, about 95% to about 99.9%, about 1% to about 95%, about 1% to about 90%, about 1% to about 85%, about 1% to about 80%, about 1% to about 75%, about 1% to about 70%, about 1% to about 65%, about 1% to about 60%, about 1% to about 55%, about 1% to about 50%, about 1% to about 45%, about 1% to about 40%, about 1% to about 35%, about 1% to about 30%, about 1% to about 25%, about 1% to about 20%, about 1% to about 15%, about 1% to about 10%, about 1% to about 5%, about 10% to about 20%, about 20% to about 30%, about 30% to about 40%, about 40% to about 50%, about 50% to about 60%, about 60% to about 70%, about 70% to about 80%, about 80% to about 90%, about 90% to about 99.9%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% by weight of the total weight of Component A, Component B, and Component C. In some or any embodiments, including any of the foregoing embodiments, the ranges provided for Component A may be combined with any of the ranges provided for Component B and/or Component C, provided that 1) the total % by weight of Components A and B amount to 100% or 2) the total % by weight of Components A, B, and C amount to 100%. In some or any embodiments, including any of the foregoing embodiments, Component A is Component A1, A2, A3, or A4. In some or any embodiments, including any of the foregoing embodiments, Component B is Component B1, B2, B3, or B4. In some or any embodiments, including any of the foregoing embodiments, Component A is Component A1 and Component B is Component B1, Component A is Component A2 and Component B is Component B2, Component A is A3 and Component B is Component B3, or Component A is A4, and Component B is Component B4. In some or any embodiments, including any of the foregoing embodiments, Component A is Component A1 and Component B is Component B1. In some or any embodiments, including any of the foregoing embodiments, Component A is Component A2 and Component B is Component B2. In some or any embodiments, including any of the foregoing embodiments, Component A is A3 and Component B is Component B3. In some or any embodiments, including any of the foregoing embodiments, Component A is A4 and Component B is Component B4.

### Medical Foods

Medical foods are foods that are specially formulated and intended for the dietary management of a disease that has distinctive nutritional needs that cannot be met by normal diet alone. Such medical foods are labeled for the dietary management of a specific medical disorder, disease or condition for which there are distinctive nutritional requirements, and are intended to be used under medical supervision. In some embodiments, the compositions described herein may be formulated as a medical food, including one or more of dietary carbohydrates, e.g., sugars and starches, dietary fats, dietary proteins, e.g., whey proteins, soy proteins and the like, vitamins, mineral, etc. In some embodiments, the medical food is a nutritionally complete formula. In some embodiments, the medical food is a nutritionally incomplete formula. The compounds described herein may be present in the medical food in any of the amounts or weight percentages described herein; an appropriate amount of the compounds described herein for the particular disease may be determined by one skilled in the art.

### Pharmaceutical Compositions

The compounds described herein can be formulated as pharmaceutical compositions by formulation with additives such as pharmaceutically acceptable excipients, pharmaceutically acceptable carriers, and pharmaceutically acceptable vehicles. Suitable pharmaceutically acceptable excipients, carriers and vehicles include processing agents and drug delivery modifiers and enhancers, such as, for example, calcium phosphate, magnesium stearate, talc, monosaccharides, disaccharides, starch, gelatin, cellulose, methyl cellulose, sodium carboxymethyl cellulose, dextrose, hydroxypropyl-β-cyclodextrin, polyvinylpyrrolidinone, low melting waxes, ion exchange resins, and the like, as well as combinations of any two or more thereof. Other suitable pharmaceutically acceptable excipients are described in “Remington's Pharmaceutical Sciences,” Mack Pub. Co., New Jersey (1991), and “Remington: The Science and Practice of Pharmacy,” Lippincott Williams & Wilkins, Philadelphia, 20th edition (2003) and 21st edition (2005), incorporated herein by reference.

A pharmaceutical composition can comprise a unit dose formulation, where the unit dose is a dose sufficient to have a therapeutic effect. The unit dose may be sufficient as a single dose to have a therapeutic effect. Alternatively, the unit dose may be a dose administered periodically in a course of treatment of a disorder.

Pharmaceutical compositions containing the compounds of the invention may be in any form suitable for the intended method of administration, including, for example, a solution, a suspension, or an emulsion. Liquid carriers are typically used in preparing solutions, suspensions, and emulsions. Liquid carriers contemplated for use in the practice of the present invention include, for example, water, saline, pharmaceutically acceptable organic solvent(s), pharmaceutically acceptable oils or fats, and the like, as well as mixtures of two or more thereof. The liquid carrier may contain other suitable pharmaceutically acceptable additives such as solubilizers, emulsifiers, nutrients, buffers, preservatives, suspending agents, thickening agents, viscosity regulators, stabilizers, and the like. Suitable organic solvents include, for example, monohydric alcohols, such as ethanol, and polyhydric alcohols, such as glycols. Suitable oils include, for example, soybean oil, sesame oil, coconut oil, olive oil, safflower oil, cottonseed oil, and the like. For parenteral administration, the carrier can also be an oily ester such as ethyl oleate, isopropyl myristate, and the like. Compositions of the present invention may also be in the form of microparticles, microcapsules, liposomal encapsulates, and the like, as well as combinations of any two or more thereof.

Time-release or controlled release delivery systems may be used, such as a diffusion controlled matrix system or an erodible system, as described for example in: Lee, “Diffusion-Controlled Matrix Systems”, pp. 155-198 and Ron and Langer, “Erodible Systems”, pp. 199-224, in “Treatise on Controlled Drug Delivery”, A. Kydonieus Ed., Marcel Dekker, Inc., New York 1992. The matrix may be, for example, a biodegradable material that can degrade spontaneously in situ and in vivo for, example, by hydrolysis or enzymatic cleavage, e.g., by proteases. The delivery system may be, for example, a naturally occurring or synthetic polymer or copolymer, for example in the form of a hydrogel. Exemplary polymers with cleavable linkages include polyesters, polyorthoesters, polyanhydrides, polysaccharides, poly(phosphoesters), polyamides, polyurethanes, poly(imidocarbonates) and poly(phosphazenes).

The compounds of the invention may be administered enterally, orally, parenterally, sublingually, by inhalation (e.g. as mists or sprays), rectally, or topically in dosage unit formulations containing conventional nontoxic pharmaceutically acceptable carriers, adjuvants, and vehicles as desired. For example, suitable modes of administration include oral, subcutaneous, transdermal, transmucosal, iontophoretic, intravenous, intraarterial, intramuscular, intraperitoneal, intranasal (e.g. via nasal mucosa), subdural, rectal, gastrointestinal, and the like, and directly to a specific or affected organ or tissue. For delivery to the central nervous system, spinal and epidural administration, or administration to cerebral ventricles, can be used. Topical administration may also involve the use of transdermal administration such as transdermal patches or iontophoresis devices. The term parenteral as used herein includes subcutaneous injections, intravenous, intramuscular, intrasternal injection, or infusion techniques. The compounds are mixed with pharmaceutically acceptable carriers, adjuvants, and vehicles appropriate for the desired route of administration. Oral administration is a preferred route of administration, and formulations suitable for oral administration are preferred formulations. The compounds described for use herein can be administered in solid form, in liquid form, in aerosol form, or in the form of tablets, pills, powder mixtures, capsules, granules, injectables, creams, solutions, suppositories, enemas, colonic irrigations, emulsions, dispersions, food premixes, and in other suitable forms. The compounds can also be administered in liposome formulations. Additional methods of administration are known in the art.

In some embodiments of the invention, especially those embodiments where a formulation is used for injection or other parenteral administration including the routes listed herein, but also including embodiments used for oral, gastric, gastrointestinal, or enteric administration, the formulations and preparations used in the methods of the invention are sterile. Sterile pharmaceutical formulations are compounded or manufactured according to pharmaceutical-grade sterilization standards (United States Pharmacopeia Chapters 797, 1072, and 1211; California Business & Professions Code 4127.7; 16 California Code of Regulations 1751, 21 Code of Federal Regulations 211) known to those of skill in the art.

Injectable preparations, for example, sterile injectable aqueous or oleaginous suspensions, may be formulated according to the known art using suitable dispersing or wetting agents and suspending agents. The sterile injectable preparation may also be a sterile injectable solution or suspension in a nontoxic parenterally acceptable diluent or solvent, for example, as a solution in propylene glycol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Solid dosage forms for oral administration may include capsules, tablets, pills, powders, and granules. In such solid dosage forms, the active compound may be admixed with at least one inert diluent such as sucrose, lactose, or starch. Such dosage forms may also comprise additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, cyclodextrins, and sweetening, flavoring, and perfuming agents.

The compounds of the present invention can also be administered in the form of liposomes. As is known in the art, liposomes are generally derived from phospholipids or other lipid substances. Liposomes are formed by mono- or multilamellar hydrated liquid crystals that are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients, and the like. The preferred lipids are the phospholipids and phosphatidyl cholines (lecithins), both natural and synthetic. Methods to form liposomes are known in the art. See, for example, Prescott, Ed., Methods in Cell Biology, Volume XIV, Academic Press, New York, N.W., p. 33 et seq (1976).

In one composition the one or more compounds is formulated as an admixture with edible oils such as corn oil, cottonseed oil, sesame oil, coconut oil and administered orally (PO), intraperitoneal (IP), or subcutaneous (SC). In another composition the one or more compounds is formulated as an admixture with propylene glycol and administered PO, IP, or SC.

In another composition the one or more compounds is adsorbed by admixture to a solid matrix from the group of calcium phosphate, calcium sulfate, starches, modified starches, microcrystalline cellulose, micro cellulose, and talcum. In some embodiments the adsorbed solid is milled into a free flowing powder and filled into hard gelatin capsules and administered orally.

In another composition the one or more compounds is admixed with edible oil or propylene glycol and filled into soft gelatin capsules and administered orally.

In another composition the one or more compounds is admixed with petroleum jelly or dissolved in dimethylsulfoxide and administered topically.

In another composition the one or more compounds is vaporized in a device such as an e-cigarette and inhaled.

In another composition the one or more compounds is emulsified as an oil in water solution. In some embodiments the emulsifier is selected from the group consisting of sodium lauryl sulfate, sorbitan monooleate, polyoxyethylene sorbitan monooleate, polysorbates, polaxomers, bile salts, glyceryl monostearate, copolymers of ethylene oxide and propylene oxide and administered topically or orally.

The invention also provides articles of manufacture and kits containing the compositions described herein. The invention also provides kits comprising a composition as described herein in a suitable container, and instructions for use. In some embodiments, the kit of the invention comprises the container described above.

The amount of active ingredient that may be combined with the carrier materials to produce a single dosage form will vary depending upon the host to which the active ingredient is administered and the particular mode of administration. It will be understood, however, that the specific dose level for any particular subject will depend upon a variety of factors including the activity of the specific compound employed, the age, body weight, body area, body mass index (BMI), general health, sex, diet, time of administration, route of administration, rate of excretion, drug combination, and the type, progression, and severity of the particular disease undergoing therapy. The pharmaceutical unit dosage chosen is usually fabricated and administered to provide a defined final concentration of drug in the blood, tissues, organs, or other targeted region of the body. The therapeutically effective amount for a given situation can be readily determined by routine experimentation and is within the skill and judgment of the ordinary clinician.

In some or any embodiments, dosages which can be used are a therapeutically effective amount within the dosage range of about 0.1 mg/kg to about 300 mg/kg body weight, or within about 1.0 mg/kg to about 100 mg/kg body weight, or within about 1.0 mg/kg to about 50 mg/kg body weight, or within about 1.0 mg/kg to about 30 mg/kg body weight, or within about 1.0 mg/kg to about 10 mg/kg body weight, or within about 10 mg/kg to about 100 mg/kg body weight, or within about 50 mg/kg to about 150 mg/kg body weight, or within about 100 mg/kg to about 200 mg/kg body weight, or within about 150 mg/kg to about 250 mg/kg body weight, or within about 200 mg/kg to about 300 mg/kg body weight, or within about 250 mg/kg to about 300 mg/kg body weight, or within about 300 mg/kg to about 350 mg/kg body weight, or within about 350 mg/kg to about 400 mg/kg body weight, or within about 400 mg/kg to about 450 mg/kg body weight, or within about 450 mg/kg to about 500 mg/kg body weight, or within about 500 mg/kg to about 550 mg/kg body weight, or within about 550 mg/kg to about 600 mg/kg body weight. Compounds of the present invention may be administered in a single daily dose, or the total daily dosage may be administered in divided dosage of two, three or four times daily.

While the compounds of the invention can be administered as the sole active pharmaceutical agent, they can also be used in combination with one or more other agents used in the treatment of the particular disorder. When additional active agents are used in combination with the compounds of the present invention, the additional active agents may generally be employed in therapeutic amounts as indicated in the Physicians' Desk Reference (PDR) 53rd Edition (1999), or such therapeutically useful amounts as would be known to one of ordinary skill in the art.

The compounds of the disclosure and the other therapeutically active agents or prophylactically effective agents can be administered at the recommended maximum clinical dosage or at lower doses. Dosage levels of the active compounds in the compositions of the disclosure may be varied so as to obtain a desired response depending on the route of administration, severity of the inflammation and/or oxidative stress exposure or disease, and the response of the patient. When administered in combination with other therapeutic or prophylactic agents, the therapeutic agents or prophylactic agents can be formulated as separate compositions that are given at the same time or different times, or the therapeutic agents or prophylactic agents can be given as a single composition.

The compounds described herein may also be administered in combination with an active agent that may cause inflammation and/or oxidative stress. In such cases, the amount of the active agent may be increased when co-administered with a compound described herein (i.e. its therapeutic window is increased as a function of co-administration with a compound described herein). Co-administration includes administration of both the compound and the other active agent in a single composition, or as separate compositions. When administered as separate compositions they may be administered simultaneously, or on different dosing schedules (e.g. one composition may be administered daily, and the other administered twice daily).

Single or multiple doses can be administered. In some embodiments, the dose is administered once, twice, three times, four times, five times, or six times. In some embodiments, the dose is administered once per day, twice per day, three times per day, or four times per day. In some embodiments, the dose is administered every hour, every two hours, every three hours, every four hours, every 6 hours, every 12 hours, or every 24 hours.

Single or multiple doses can be administered prior to or after indications of inflammation and/or oxidative stress. In some embodiments, the dose is administered once, twice, three times, four times, five times, or six times during or after appearance of indicators of inflammation and/or oxidative stress. In some embodiments, the dose is administered once per day, twice per day, three times per day, or four times per day during or after appearance of indicators of inflammation and/or oxidative stress. In some embodiments, the dose is administered every hour, every two hours, every three hours, every four hours, every 6 hours, every 12 hours, or every 24 hours during or after appearance of indicators of inflammation and/or oxidative stress.

Single or multiple doses can be administered before appearance of inflammation and/or oxidative stress. In some embodiments, the dose is administered once, twice, three times, four times, five times, or six times before appearance of inflammation and/or oxidative stress. In some embodiments, the dose is administered once per day, twice per day, three times per day, or four times per day before appearance of inflammation and/or oxidative stress. In some embodiments, the dose is administered every hour, every two hours, every three hours, every four hours, every 6 hours, every 12 hours, or every 24 hours before appearance of inflammation and/or oxidative stress. In some embodiments, the dose is administered about 30 minutes before appearance of inflammation and/or oxidative stress, about 1 hour before appearance of inflammation and/or oxidative stress, about 2 hours before appearance of inflammation and/or oxidative stress, about 3 hours before appearance of inflammation and/or oxidative stress, about 4 hours before appearance of inflammation and/or oxidative stress, about 6 hours before appearance of inflammation and/or oxidative stress, about 8 hours before appearance of inflammation and/or oxidative stress, about 10 hours before appearance of inflammation and/or oxidative stress, about 12 hours before appearance of inflammation and/or oxidative stress, and/or about 24 hours before appearance of inflammation and/or oxidative stress. In some embodiments, the dose is administered about 4 hours before appearance of inflammation and/or oxidative stress. In some embodiments, the dose is administered about 24 hours before appearance of inflammation and/or oxidative stress. In some embodiments, the dose is administered about 4 hours and about 24 hours before appearance of inflammation and/or oxidative stress.

While the compounds of the disclosure can be administered as the sole active pharmaceutical agent, they can also be used in combination with one or more other agents used in the treatment of or prophylaxis against inflammation and/or oxidative stress. In some embodiments, the compound(s) of the disclosure are administered as the sole active pharmaceutical agent that is present in a therapeutically effective amount.

The one or more inflammation and/or oxidative stress protective agent can be administered about 0 to about 48 hours after appearance of inflammation and/or oxidative stress in single or multiple doses. The one or more inflammation and/or oxidative stress protective agent can be administered about 0 to about 24 hours after appearance of inflammation and/or oxidative stress in single or multiple doses. The one or more inflammation and/or oxidative stress protective agent can be administered about 0 to about 12 hours after appearance of inflammation and/or oxidative stress in single or multiple doses. The one or more inflammation and/or oxidative stress protective agent can be administered about 0 to about 4 hours after appearance of inflammation and/or oxidative stress in single or multiple doses. The one or more inflammation and/or oxidative stress protective agent can be administered about 0 to about 1 hour after appearance of inflammation and/or oxidative stress in single or multiple doses. The one or more protective agent can be administered about 0 to about 30 minutes after appearance of inflammation and/or oxidative stress in single or multiple doses. The one or more protective agent can be administered about 0 to about 15 minutes after appearance of inflammation and/or oxidative stress in single or multiple doses. The one or more inflammation and/or oxidative stress protective agent can be administered about 0 to about 10 minutes after appearance of inflammation and/or oxidative stress in single or multiple doses. The one or more inflammation and/or oxidative stress protective agent can be administered about 0 to about 5 minutes after appearance of inflammation and/or oxidative stress in single or multiple doses. The one or more inflammation and/or oxidative stress protective agent can be administered about 5 minutes to about 48 hours after appearance of inflammation and/or oxidative stress in single or multiple doses. The one or more inflammation and/or oxidative stress protective agent can be administered about 5 minutes to about 24 hours after appearance of inflammation and/or oxidative stress in single or multiple doses. The one or more inflammation and/or oxidative stress protective agent can be administered about 5 minutes to about 12 hours after appearance of inflammation and/or oxidative stress in single or multiple doses. The one or more inflammation and/or oxidative stress protective agent can be administered about 5 minutes to about 4 hours after appearance of inflammation and/or oxidative stress in single or multiple doses. The one or more inflammation and/or oxidative stress protective agent can be administered about 5 minutes to about 2 hours after appearance of inflammation and/or oxidative stress in single or multiple doses. The one or more inflammation and/or oxidative stress protective agent can be administered about 5 minutes to about 1 hour after appearance of inflammation and/or oxidative stress in single or multiple doses. The one or more inflammation and/or oxidative stress protective agent can be administered about 30 minutes to about 48 hours after appearance of inflammation and/or oxidative stress in single or multiple doses. The one or more inflammation and/or oxidative stress protective agent can be administered about 30 minutes to about 24 hours after appearance of inflammation and/or oxidative stress in single or multiple doses. The one or more inflammation and/or oxidative stress protective agent can be administered about 30 minutes to about 12 hours after appearance of inflammation and/or oxidative stress in single or multiple doses. The one or more inflammation and/or oxidative stress protective agent can be administered about 30 minutes to about 4 hours after appearance of inflammation and/or oxidative stress in single or multiple doses. The one or more inflammation and/or oxidative stress protective agent can be administered about 30 minutes to about 2 hours after appearance of inflammation and/or oxidative stress in single or multiple doses. The one or more inflammation and/or oxidative stress protective agent can be administered about 30 minutes to about 1 hour after appearance of inflammation and/or oxidative stress in single or multiple doses. The one or more inflammation and/or oxidative stress protective agent can be administered about 5 minutes, about 10 minutes, about 20 minutes, about 30 minutes, about 1 hour, about 1.5 hours, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, bout 16 hours, about 17 hours, about 18 hours, about 20 hours, about 22 hours, about 24 hours, about 30 hours, about 36 hours, about 42 hours, and/or about 48 hours after appearance of inflammation and/or oxidative stress.

The one or more inflammation and/or oxidative stress protective agent can be administered about 0 to about 48 hours before appearance of inflammation and/or oxidative stress in single or multiple doses. The one or more inflammation and/or oxidative stress protective agent can be administered about 0 to about 24 hours before appearance of inflammation and/or oxidative stress in single or multiple doses. The one or more inflammation and/or oxidative stress protective agent can be administered about 0 to about 12 hours before appearance of inflammation and/or oxidative stress in single or multiple doses. The one or more inflammation and/or oxidative stress protective agent can be administered about 0 to about 4 hours before appearance of inflammation and/or oxidative stress in single or multiple doses. The one or more inflammation and/or oxidative stress protective agent can be administered about 0 to about 1 hour before appearance of inflammation and/or oxidative stress in single or multiple doses. The one or more inflammation and/or oxidative stress protective agent can be administered about 0 to about 30 minutes before appearance of inflammation and/or oxidative stress in single or multiple doses. The one or more inflammation and/or oxidative stress protective agent can be administered about 0 to about 15 minutes before appearance of inflammation and/or oxidative stress in single or multiple doses. The one or more inflammation and/or oxidative stress protective agent can be administered about 0 to about 10 minutes before appearance of inflammation and/or oxidative stress in single or multiple doses. The one or more inflammation and/or oxidative stress protective agent can be administered about 0 to about 5 minutes before appearance of inflammation and/or oxidative stress in single or multiple doses. The one or more inflammation and/or oxidative stress protective agent can be administered about 5 minutes to about 48 hours before appearance of inflammation and/or oxidative stress in single or multiple doses. The one or more inflammation and/or oxidative stress protective agent can be administered about 5 minutes to about 24 hours before appearance of inflammation and/or oxidative stress in single or multiple doses. The one or more inflammation and/or oxidative stress protective agent can be administered about 5 minutes to about 12 hours before appearance of inflammation and/or oxidative stress in single or multiple doses. The one or more inflammation and/or oxidative stress protective agent can be administered about 5 minutes to about 4 hours before appearance of inflammation and/or oxidative stress in single or multiple doses. The one or more inflammation and/or oxidative stress protective agent can be administered about 5 minutes to about 2 hours before appearance of inflammation and/or oxidative stress in single or multiple doses. The one or more inflammation and/or oxidative stress protective agent can be administered about 5 minutes to about 1 hour before appearance of inflammation and/or oxidative stress in single or multiple doses. The one or more inflammation and/or oxidative stress protective agent can be administered about 30 minutes to about 48 hours before appearance of inflammation and/or oxidative stress in single or multiple doses. The one or more inflammation and/or oxidative stress protective agent can be administered about 30 minutes to about 24 hours before appearance of inflammation and/or oxidative stress in single or multiple doses. The one or more inflammation and/or oxidative stress protective agent can be administered about 30 minutes to about 12 hours before appearance of inflammation and/or oxidative stress in single or multiple doses. The one or more inflammation and/or oxidative stress protective agent can be administered about 30 minutes to about 4 hours before appearance of inflammation and/or oxidative stress in single or multiple doses. The one or more inflammation and/or oxidative stress protective agent can be administered about 30 minutes to about 2 hours before appearance of inflammation and/or oxidative stress in single or multiple doses. The one or more inflammation and/or oxidative stress protective agent can be administered about 30 minutes to about 1 hour before appearance of inflammation and/or oxidative stress in single or multiple doses. The one or more inflammation and/or oxidative stress protective agent can be administered about 5 minutes, about 10 minutes, about 20 minutes, about 30 minutes, about 1 hour, about 1.5 hours, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, bout 16 hours, about 17 hours, about 18 hours, about 20 hours, about 22 hours, about 24 hours, about 30 hours, about 36 hours, about 42 hours, and/or about 48 hours before appearance of inflammation and/or oxidative stress.

### Assessment and Efficacy of Therapy

The utility of the compounds, compositions, and methods of the present disclosure for inflammation and/or oxidative stress protection may be demonstrated both in vitro and in vivo. For example, the ability of cultured cells to form clones (colonies) may be evaluated as a function of inflammation and/or oxidative stress. Cells are either not treated or are treated with a compound or composition of the disclosure at a certain time (in some embodiments, 30 minutes) prior to exposure to factors which may result in inflammation and/or oxidative stress. The degree of retention of ability to form clones after exposure, in comparison to untreated cells, is directly related to the protective effect of the compounds or compositions.

In vivo, the utility of the compounds, compositions, and methods of the present disclosure for influence on inflammation and/or oxidative stress may be evaluated by mice exposed to conditions commensurate with the manifestation of inflammation and/or oxidative stress. Animals, either pre-dosed with a compound or composition disclosed herein, or not dosed (i.e., control animals), are subjected to conditions or materials which can result in inflammation and/or oxidative stress. In some embodiments, control animals are expected to survive about 12-15 days. The dosed animals response, in comparison to the controls, is directly related to the protective effect of the compound or composition administered. Additionally, blood collection for metabolomics analyses can be conducted at intervals during testing, while tissue sampling can be conducted at testing end point. Evaluation of blood and tissue samples can be compared to untreated control animals for efficacy.

### Preparation of Compounds of the Invention

In general, the nomenclature used in this Application was generated with the help of naming package within the ChemOffice®. version 11.0 suite of programs by CambridgeSoft Corp (Cambridge, Mass.).

The compounds of this disclosure can be prepared from readily available starting materials using general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Preparation of the compounds disclosed herein is described in co-assigned US Patent Application Publications No. 2006/0281809, 2007/0072943, and 2010/0105930, and PCT publications WO 2013/013078, WO2017/106803, and WO2017/106786. Additional methods are described in U.S. Provisional Application No. 62/485,739, titled “VITAMIN E COMPOSITIONS AND METHODS OF USE THEREFOR”, filed on Apr. 14, 2017, U.S. Provisional Application No. 62/488,643, titled “VITAMIN E COMPOSITIONS AND METHODS OF USE THEREFOR”, filed on Apr. 21, 2017, U.S. Nonprovisional Application No. ______, titled “VITAMIN E COMPOSITIONS AND METHODS OF USE THEREFOR”, filed on Apr. 14, 2018, U.S. Provisional Application No. 62/635,470 filed on Feb. 26, 2018, and titled “METHODS, COMPOSITIONS AND SYSTEMS FOR MONITORING AND INFLUENCING FERROPTOSIS AND INFLAMMATION IN BIOLOGICAL SYSTEMS”, U.S. Non-Provisional Application titled “VITAMIN E COMPOSITIONS AND METHODS OF USE THEREFOR”, filed on Apr. 14 2018, all by the same Applicant, and which are incorporated by reference herein in their entireties.

Furthermore, the compounds of this disclosure will typically contain one or more chiral centers. Accordingly, if desired, such compounds can be prepared or isolated as pure stereoisomers, i.e., as individual enantiomers or diastereomers, or as stereoisomer-enriched mixtures. All such stereoisomers (and enriched mixtures) are included within the scope of this disclosure, unless otherwise indicated. Pure stereoisomers (or enriched mixtures) may be prepared using, in some embodiments, optically active starting materials or stereoselective reagents well-known in the art. Alternatively, racemic mixtures of such compounds can be separated using, in some embodiments, chiral column chromatography, chiral resolving agents and the like. Advantages associated with the use of pure stereoisomers is outlined in co-pending U.S. Nonprovisional application No. ______, titled “VITAMIN E COMPOSITIONS AND METHODS OF USE THEREFOR”, filed on Apr. 14, 2018and incorporated herein by reference.

The disclosure is further described by the following non-limiting examples and embodiments.

### EXAMPLES

The following examples are given for the purpose of illustrating various embodiments of the present disclosure and are not meant to limit the present disclosure in any fashion. The present examples; along with the methods described herein are presently representative of preferred cases; are exemplary; and are not intended as limitations on the scope of the present disclosure. Changes therein and other uses which are encompassed within the spirit of the present disclosure as defined by the scope of the claims will occur to those skilled in the art.

### Example 1

### Assessment of Enzyme Concentration in Response to Induced Liver Damage

Compounds described herein were tested to determine their effects on liver enzyme concentrations following introduction of APAP in sufficient quantity to cause liver trauma.

### Experimental Design

Male C57BL/6 mice (Charles River Laboratories), aged 10-12 weeks, were randomly and prospectively assigned to receive treatment with either a test compound or vehicle only. Prior to commencement of testing, the animals had been pre-dosed with a test compound of either EPI-A0001 ((RRR)-alpha tocopherol quinone) or EPI-743 (REE)-alpha tocotrienol quinone) via subcutaneous injection for 4 days at levels of 300 mg/kg body weight dose(s). One additional test compound was administrated at 4 hours prior to commencement of testing. The mice were fasted for 12 hours prior to being challenged with 300 mg/kg body weight with APAP or an equal volume of the dosing vehicle (sterile saline) via intraperitoneal injection.

### Animal Housing and Environment

The animals were housed in disposable cages with sterile wood chip bedding, food, and water. The mice were acclimated for at least 3 days and given food and tap water ad libitum. The animals were examined prior to initiation of the study to assure adequate health and suitability. Animals that were found to be diseased or unsuitable were not assigned to the study.

During the course of the study, 12-hour light/12-hour dark cycles were maintained. A nominal temperature range of 20-23° C. with relative humidity between 30% and 70% were also maintained. LabDiet 5053-certified PicoLab Rodent Diet and sterile water were provided ad libitum.

### Statistical Analysis

Statistical differences between treatment groups were determined using Kruskal-Wallis non-parametric test with Dunn's test for multiple comparisons.

### Test Article Administration

The animals were each administered with a single subcutaneous (SC) 300 mg/kg body weight dose of APAP or vehicle only formulation as described herein. All animals were dosed at approximately the same time on the dosing day (±1 hour). Subcutaneous doses were administered via bolus injection between the skin and underlying layers of tissue in the scapular region on the back of each animal. The hair was not clipped from the injection site prior to dosing. The injection site was monitored for necrosis and other changes to the skin and hair.

At the time of sample collection, livers were collected for histopathology and metabolomics analyses and blood was collected for metabolomics analyses or separated into serum for the liver enzyme (AST, ALT) analyses.

### Test Summary

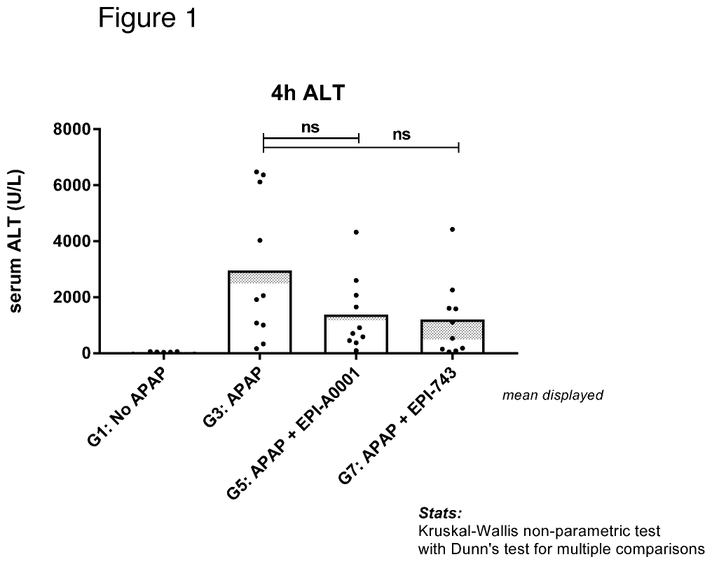

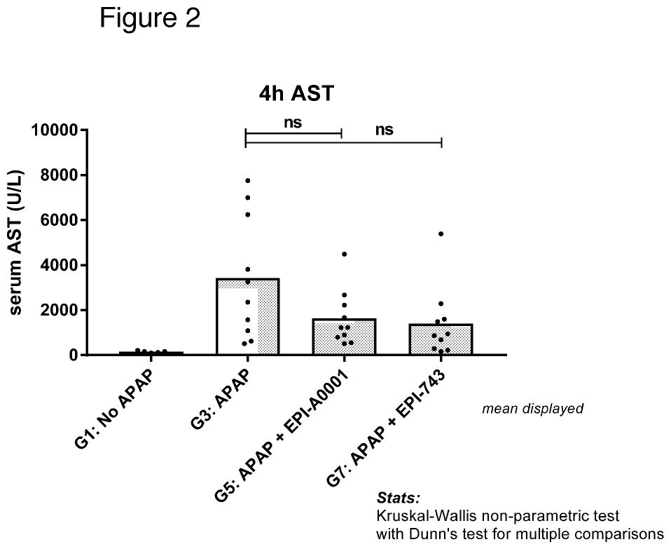

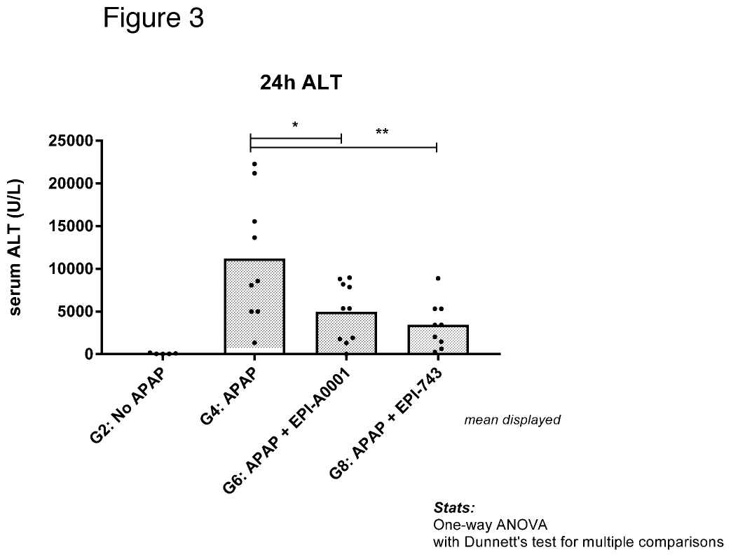

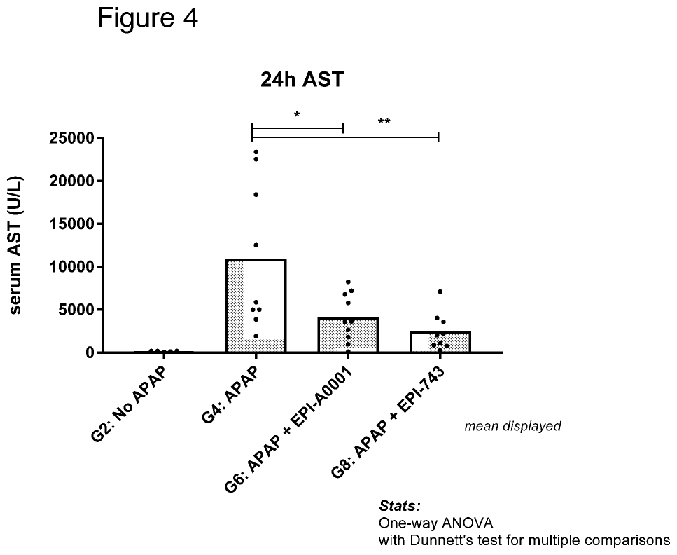

Test FIGS. 1 and 2 show the four hour serum measurements for liver enzymes ALT and AST, respectively, for the control, APAP, APAP+EPI-A0001, and APAP +EPI-743. Test FIGS. 3 and 4 show the 24 hour serum measurements for ALT and AST, respectively, for the control, APAP, APAP +EPI-A0001, and APAP+EPI-743.

Test FIG. 1 shows the measured levels for ALT liver enzyme, where each data point represents a single animal. The shaded rectangles represent the mean level of ALT concentration, where G1 (the control) indicates a zero concentration of ALT. The liver trauma is induced via APAP introduction in G3, and the mean level of ALT measured reaches approximately 3000 U/L for ALT and 3200 U/L for AST. Note the reduction in mean serum concentrations in both G5 and G7, and in particular 40% of the data points (4 of 10) in G7 are near zero. This same phenomenon is seen in Test FIG. 2, where 30% (3 of 10) of the AST levels are near zero. Test FIGS. 3 and 4 show similar results at 24 hours as the 4 hour results of Test FIGS. 1 and 2. Again, note the reduction in mean serum concentration level for G6 and G8 over G4, and several data points at or near zero for G8 in ALT and AST.

### Example 2

### Assessment of Suppressive Activity on Release of Cytokines for Endothelial Cells

### Experimental Protocol

Human Aortic Endothelial Cells (HAEC) stimulated with IL-1β result in the secretion of pro-inflammatory cytokines IL-6, IL-8 and MCP-1 as described in Wu et al., Atherosclerosis 147, 297-307 (1999). The detection of these secreted cytokines in the presence or absence of compounds was performed using Meso Scale technology.

HAEC cells from two donors (Catalog no. 2535), EGM-2 media with BulletKit supplements (Catalog no. 3162) were purchased from Lonza. IL-1β (Catalog no. 200-01B) was purchased from Peprotech. Custom MSD U-plex plates with assays to detect IL-6, IL-8 and TNF-α (Catalog no. K15067L) were purchased from Meso Scale Discovery. The cells were grown in tissue culture-treated flasks according to Lonza's instructions.

Test samples were supplied in 1.5 mL glass vials. The compounds were diluted with an appropriate volume of DMSO to result in a 10 mM stock solution. Once dissolved, they were stored at −20° C.

Test samples were screened according to the following protocol:

HAEC cells were cultured and used at passage 6. Cells were seeded in clear bottom, black wall 96-well tissue culture-treated polystyrene plates by resuspending a cell suspension at a density of 150,000 cells/mL in growth medium, then dispensing 100 microliters of cell suspension per well using an electronic multichannel pipette, corresponding to 15,000 cells/well (one plate per donor). The cell-containing plates were incubated 3 hours at 37° C. in an atmosphere with 95% humidity and 5% CO₂ to allow attachment of the cells to the culture plate.

Test compounds were then added to varying final concentrations using the Tecan D300e Digital Dispenser, with subsequent back-filling with DMSO diluent to a final concentration of 0.6% (v/v). Cell plates were incubated for 20 hours at 37° C. in an atmosphere of 5% CO₂ and 95% humidity.

IL-1β was then added to a final concentration of 10 nanograms/mL and then cell plates were incubated at 37° C. in an atmosphere of 5% CO₂ and 95% humidity for 6 hours. Media was collected in a separate polypropylene plate, foil sealed and frozen at −80° C. for up to 96 hours.

Frozen media samples were thawed on ice and IL-6, IL-8 and TNF-alpha were measured using the Meso Scale multiplex assay according to the manufacturer's instructions. Included on each MSD plate was a dilution of IL-6, IL-8 and TNF-alpha standards to accurately quantify the secreted proteins in the media.

### Test Summary

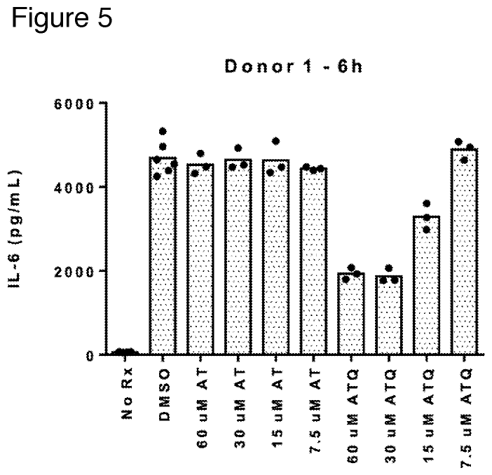

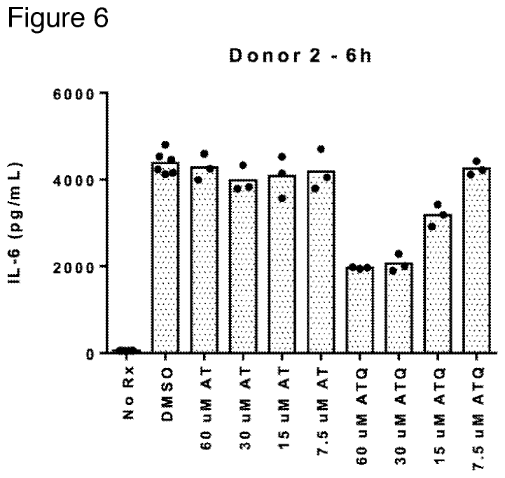

FIGS. 5 and 6 present results for IL-6 (Interleukin-6) suppression by various test compounds, including the quinone A0001 (ATQ in these figures). Note the dosing dependent effect on IL-6 suppression using A0001, where 60 μM and 30 μM dosing levels of A0001 result in excess of 50% drop in levels of IL-6 in the DMSO control and various dosing levels of alpha-tocopherol (AT in these figures). The suppression of IL-6 levels using the 15 μM dosing of A0001 is not as dramatic as displayed by the 60 μM and 30 μM dosing levels of A0001. At 7.5 μM of A0001 the IL-6 suppression is no longer evident.

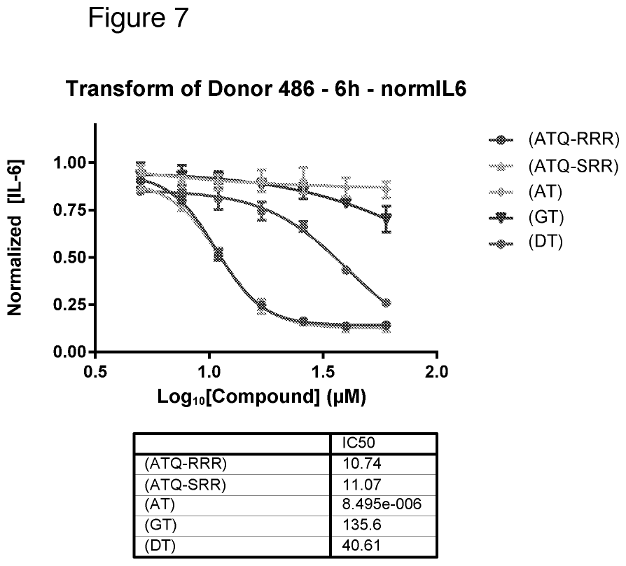

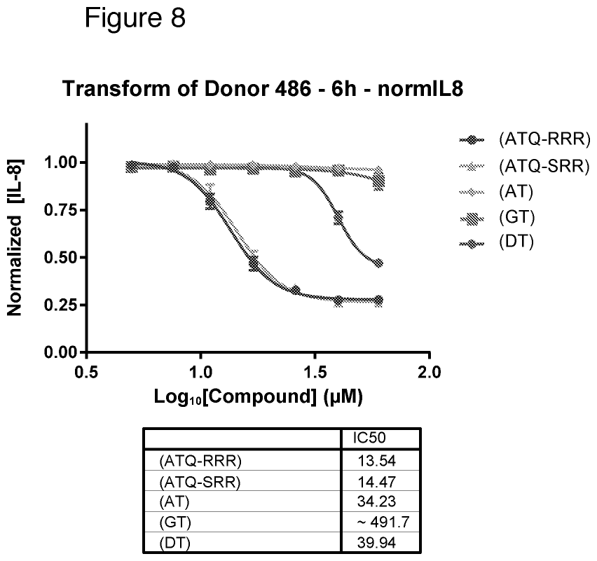

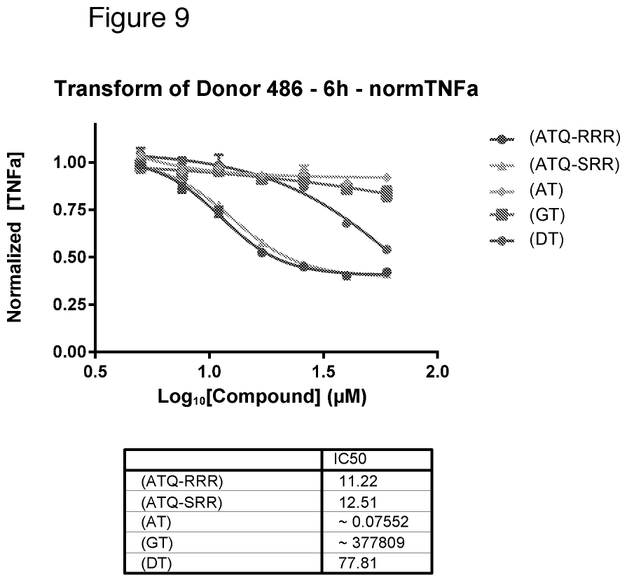

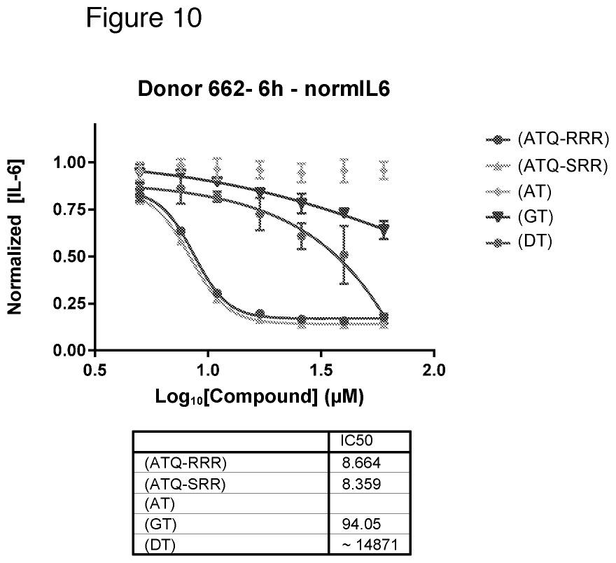

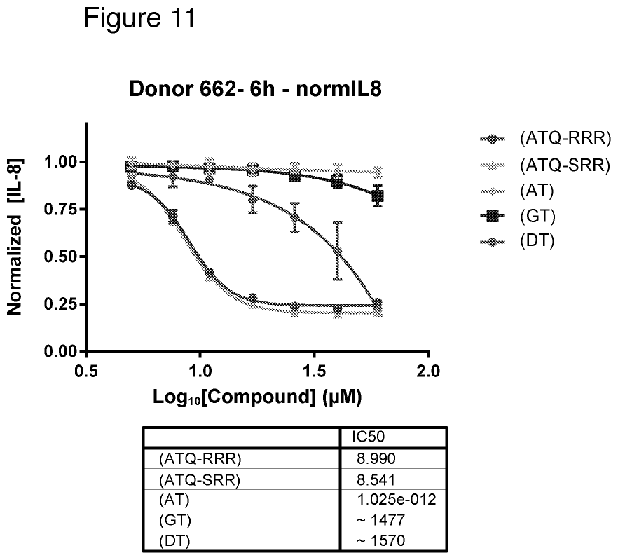

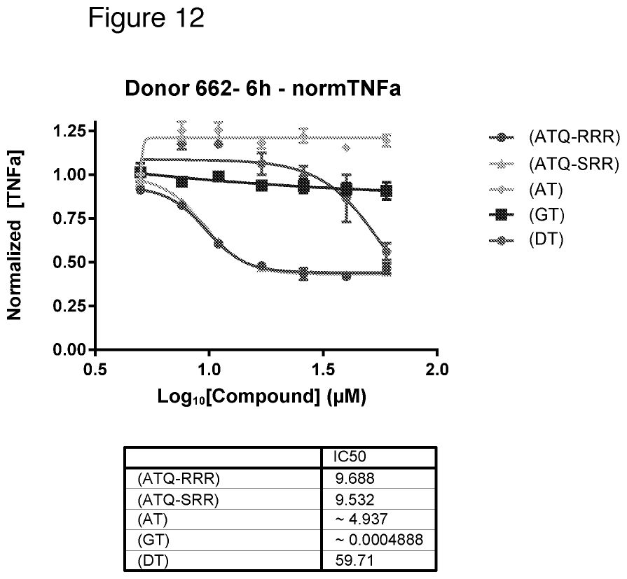

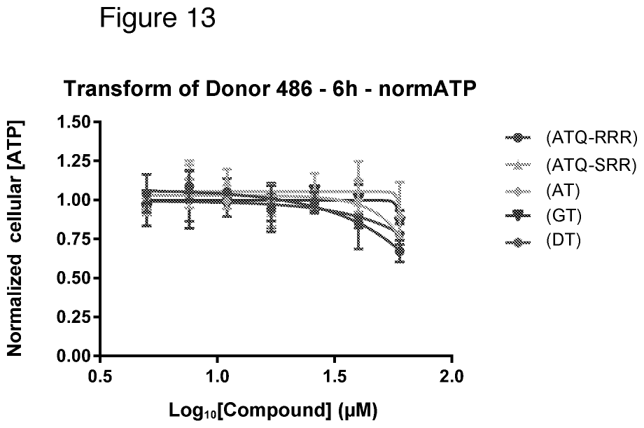

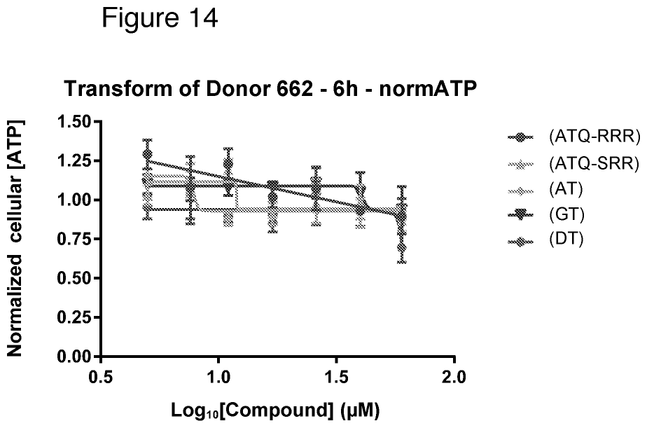

FIGS. 7-14 present an expanded array of pro-inflammatory cytokines, including IL-6, IL-8, and TNFₐ (Tumor Necrosis Factor alpha). The results are consistent with that presented in FIGS. 5 and 6. Again, the quinone A0001 exhibited a dose dependent suppression of the various cytokines, and a small variation is exhibited between the A0001-RRR and A0001-SRR diastereomers. In these figures, GT is gamma-tocopherol, and DT is delta-tocopherol. FIGS. 13 and 14 exhibit only a slight effect of cell viability from the A0001 at the higher dosing levels, via normalized ATP levels between the two donors (486 & 662).

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method of treating or protecting against inflammation in a biological system comprising: administering a therapeutically effective amount or prophylactically effective amount of a compound to a biological system in need thereof, wherein the compound is according to the formula:
(a) 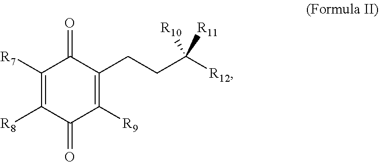
or the hydroquinone form thereof;
wherein:
R₇, R₈, and R₉ are independently hydrogen, C₁-C₆ alkyl, or C₁-C₆ alkoxy;
R₁₀ and R₁₁ are independently selected from the group consisting of hydrogen, methyl, and hydroxyl, with the proviso that only one of R₁₀ and R₁₁ may be hydroxyl; and
R₁₂ is a C₁-C₁₃-alkyl or C₂-C₁₃-alkenyl group, wherein the C₁-C₁₃-alkyl and C₂-C₁₃-alkenyl groups are optionally substituted with one or more groups selected from the group consisting of: C₁-C₃ alkyl, halo, C₁-C₆ alkoxy, C₁-C₆ haloalkoxy, hydroxyl, and carboxylic acid; or
(b) 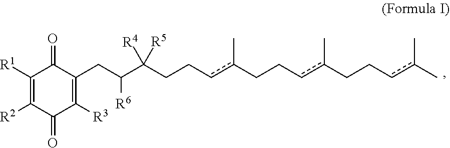
or the hydroquinone form thereof;
wherein:
each bond indicated with a dashed line is independently a single bond or a double bond;
R¹, R², and R³ are independently selected from H, (C₁-C₄)-alkyl, (C₁-C₄)-haloalkyl, —CN, —F, —Cl, —Br, and —I; and
R⁴ and R⁵ are independently selected from hydroxy and (C₁-C₄)-alkyl, and R⁶ is hydrogen; or
R⁴ is (C₁-C₄)-alkyl, and R⁵ and R⁶ are hydrogen; or
R⁴ is (C₁-C₄)-alkyl, and R⁵ and R⁶ together form the second bond of a double bond between the carbon atoms to which they are attached;
or a stereoisomer, mixtures of stereoisomers, salt, phosphate substituted form, crystalline form, non-crystalline form, isotopologue, deuterated form, hydrate, or solvate thereof.

2-19. (canceled)

20. The method of claim 1 wherein the compound is selected from the group consisting of alpha-tocotrienol quinone, beta-tocotrienol quinone, gamma-tocotrienol quinone, delta-tocotrienol quinone, alpha-tocopherol quinone, beta-tocopherol quinone, gamma-tocopherol quinone, delta-tocopherol quinone, and the corresponding hydroquinone forms thereof, and stereoisomers, mixtures of stereoisomers, hydrates, and solvates thereof.

21-65. (canceled)

66. A method of reducing drug-induced solid organ injury, comprising co-administering to a subject in need thereof: (a) a drug that may cause solid organ injury, and (b) a compound comprising the formula:
(a) 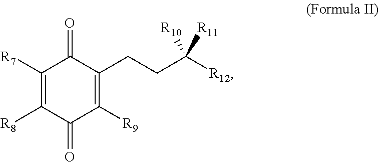
or the hydroquinone form thereof;
wherein:
R₇, R8, and R₉ are independently hydrogen, C₁-C₆ alkyl, or C₁-C₆ alkoxy;
R₁₀ and R₁₁ are independently selected from the group consisting of hydrogen, methyl, and hydroxyl, with the proviso that only one of R₁₀ and R₁₁ may be hydroxyl; and
R₁₂ is a C₁-C₁₃-alkyl or C₂-C₁₃-alkenyl group, wherein the C₁-C₁₃-alkyl or C₂-C₁₃-alkenyl groups are optionally substituted with one or more groups selected from the group consisting of: C₁-C₃ alkyl, halo, C₁-C₆ alkoxy, C₁-C₆ haloalkoxy, hydroxyl, and carboxylic acid; or
(b) 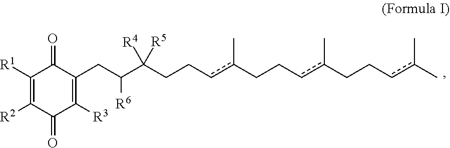
or the hydroquinone form thereof;
wherein:
each bond indicated with a dashed line is independently a single bond or a double bond;
R¹, R², and R³ are independently selected from H, (C₁-C₄)-alkyl, (C₁-C₄)-haloalkyl, —CN, —F, —Cl, —Br, and —I; and
R⁴ and R⁵ are independently selected from hydroxy and (C₁-C₄)-alkyl, and R⁶ is hydrogen; or
R⁴ is (C₁-C₄)-alkyl, and R⁵ and R⁶ are hydrogen; or
R⁴ is (C₁-C₄)-alkyl, and R⁵ and R⁶ together form the second bond of a double bond between the carbon atoms to which they are attached;
or a stereoisomer, mixtures of stereoisomers, salt, phosphate substituted form, crystalline form, non-crystalline form, isotopologue, deuterated form, hydrate, or solvate thereof.

67-88. (canceled)

89. The method of claim 66 wherein the compound is selected from the group consisting of alpha-tocotrienol quinone, beta-tocotrienol quinone, gamma-tocotrienol quinone, delta-tocotrienol quinone, alpha-tocopherol quinone, beta-tocopherol quinone, gamma-tocopherol quinone, delta-tocopherol quinone, and the corresponding hydroquinone forms thereof, and stereoisomers, mixtures of stereoisomers, hydrates, and solvates thereof.

90-123. (canceled)

124. A method of treating inflammation, comprising:
(a) measuring the levels of one or more biomarker(s) selected from the group consisting of: ALT, AST, IL-6, IL-8, MCP-1, and TNF-α in a subject, and
(b) administering to the subject a compound when the biomarker(s) indicates inflammation, wherein the compound is according to the formula:
(a) 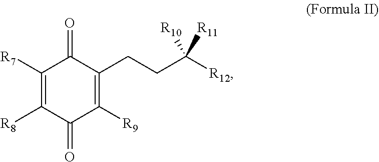
or the hydroquinone form thereof;
wherein:
R₇, R₈, and R₉ are independently hydrogen, C₁-C₆ alkyl, or C₁-C₆ alkoxy;
R₁₀ and R₁₁ are independently selected from the group consisting of hydrogen, methyl, and hydroxyl, with the proviso that only one of R₁₀ and R₁₁ may be hydroxyl; and
R₁₂ is a C₁-C₁₃-alkyl or C₂-C₁₃-alkenyl group, wherein the C₁-C₁₃-alkyl and C₂-C₁₃-alkenyl groups are optionally substituted with one or more groups selected from the group consisting of: C₁-C₃ alkyl, halo, C₁-C₆ alkoxy, C₁-C₆ haloalkoxy, hydroxyl, and carboxylic acid; or
(b) 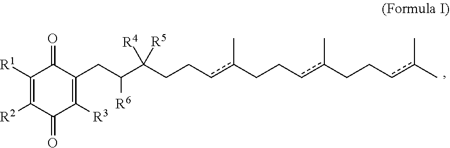
or the hydroquinone form thereof;
wherein:
each bond indicated with a dashed line is independently a single bond or a double bond;
R¹, R², and R³ are independently selected from H, (C₁-C₄)-alkyl, (C₁-C₄)-haloalkyl, —CN, —F, —Cl, —Br, and —I; and
R⁴ and R⁵ are independently selected from hydroxy and (C₁-C₄)-alkyl, and R⁶ is hydrogen; or
R⁴ is (C₁-C₄)-alkyl, and R⁵ and R⁶ are hydrogen; or
R⁴ is (C₁-C₄)-alkyl, and R⁵ and R⁶ together form the second bond of a double bond between the carbon atoms to which they are attached;
or a stereoisomer, mixtures of stereoisomers, salt, phosphate substituted form, crystalline form, non-crystalline form, isotopologue, deuterated form, hydrate, or solvate thereof.

125-130. (canceled)

131. A method of reducing alanine transaminase and/or aspartate transaminase in a biological system comprising: administering a therapeutically effective amount or prophylactically effective amount of a compound to a biological system in need thereof, wherein the compound is according to the formula:
(a) 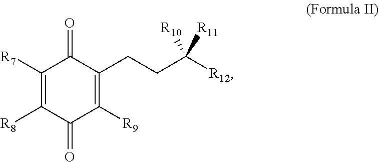
or the hydroquinone form thereof;
wherein:
R₇, R₈, and R₉ are independently hydrogen, C₁-C₆ alkyl, or C₁-C₆ alkoxy;
R₁₀ and R₁₁ are independently selected from the group consisting of hydrogen, methyl, and hydroxyl, with the proviso that only one of R₁₀ and R₁₁ may be hydroxyl; and
R₁₂ is a C₁-C₁₃-alkyl or C₂-C₁₃-alkenyl group, wherein the C₁-C₁₃-alkyl and C₂-C₁₃-alkenyl groups are optionally substituted with one or more groups selected from the group consisting of: C₁-C₃ alkyl, halo, C₁-C₆ alkoxy, C₁-C₆ haloalkoxy, hydroxyl, and carboxylic acid; or
(b) 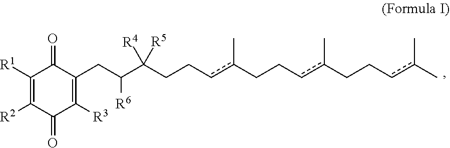
or the hydroquinone form thereof;
wherein:
each bond indicated with a dashed line is independently a single bond or a double bond;
R¹, R², and R³ are independently selected from H, (C₁-C₄)-alkyl, (C₁-C₄)-haloalkyl, —CN, —F, —Cl, —Br, and —I; and
R⁴ and R⁵ are independently selected from hydroxy and (C₁-C₄)-alkyl, and R⁶ is hydrogen; or
R⁴ is (C₁-C₄)-alkyl, and R⁵ and R⁶ are hydrogen; or
R⁴ is (C₁-C₄)-alkyl, and R⁵ and R⁶ together form the second bond of a double bond between the carbon atoms to which they are attached;
or a stereoisomer, mixtures of stereoisomers, salt, phosphate substituted form, crystalline form, non-crystalline form, isotopologue, deuterated form, hydrate, or solvate thereof.

132. The method of claim 131, wherein the compound is selected from the group consisting of alpha-tocotrienol quinone, beta-tocotrienol quinone, gamma-tocotrienol quinone, delta-tocotrienol quinone, alpha-tocopherol quinone, beta-tocopherol quinone, gamma-tocopherol quinone, delta-tocopherol quinone, and the corresponding hydroquinone forms thereof, and stereoisomers, mixtures of stereoisomers, hydrates, and solvates thereof.

133. A method of reducing or suppressing one or more reducing a pro-inflammatory cytokine in a biological system comprising: administering a therapeutically effective amount or prophylactically effective amount of a compound to a biological system in need thereof, wherein the compound is according to the formula:
(a) 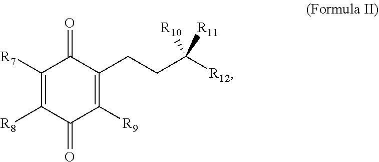
or the hydroquinone form thereof;
wherein:
R₇, R₈, and R₉ are independently hydrogen, C₁-C₆ alkyl, or C₁-C₆ alkoxy;
R₁₀ and R₁₁ are independently selected from the group consisting of hydrogen, methyl, and hydroxyl, with the proviso that only one of R₁₀ and R₁₁ may be hydroxyl; and
R₁₂ is a C₁-C₁₃-alkyl or C₂-C₁₃-alkenyl group, wherein the C₁-C₁₃-alkyl and C₂-C₁₃-alkenyl groups are optionally substituted with one or more groups selected from the group consisting of: C₁-C₃ alkyl, halo, C₁-C₆ alkoxy, C₁-C₆ haloalkoxy, hydroxyl, and carboxylic acid; or
(b) 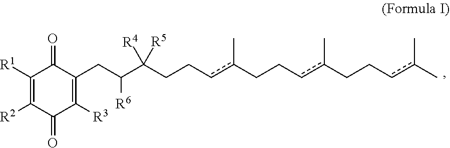
or the hydroquinone form thereof;
wherein:
each bond indicated with a dashed line is independently a single bond or a double bond;
R¹, R², and R³ are independently selected from H, (C₁-C₄)-alkyl, (C₁-C₄)-haloalkyl, —CN, —F, —Cl, —Br, and —I; and
R⁴ and R⁵ are independently selected from hydroxy and (C₁-C₄)-alkyl, and R⁶ is hydrogen; or
R⁴ is (C₁-C₄)-alkyl, and R⁵ and R⁶ are hydrogen; or
R⁴ is (C₁-C₄)-alkyl, and R⁵ and R⁶ together form the second bond of a double bond between the carbon atoms to which they are attached;
or a stereoisomer, mixtures of stereoisomers, salt, phosphate substituted form, crystalline form, non-crystalline form, isotopologue, deuterated form, hydrate, or solvate thereof.

134. The method of claim 133, wherein the compound is selected from the group consisting of alpha-tocotrienol quinone, beta-tocotrienol quinone, gamma-tocotrienol quinone, delta-tocotrienol quinone, alpha-tocopherol quinone, beta-tocopherol quinone, gamma-tocopherol quinone, delta-tocopherol quinone, and the corresponding hydroquinone forms thereof, and stereoisomers, mixtures of stereoisomers, hydrates, and solvates thereof.
